(19) United States
(12) Patent Application Publication
(10) Pub. No.: US 2015/0174207 A1
(43) Pub. Date: Jun. 25, 2015

(54) METHODS FOR TREATING CANCER WITH GHRH AGONISTS

(71) Applicants: UNIVERSITY OF MIAMI, Miami, FL (US); UNITED STATES OF AMERICA, REPRESENTED BY THE DEPARTMENT OF VETERANS AFFAIRS, Washington, DC (US)

(72) Inventors: Andrew V. SCHALLY, Miami, FL (US); Miklos JASBERENYI, Miami, FL (US)

(21) Appl. No.: 14/582,866

(22) Filed: Dec. 24, 2014

Related U.S. Application Data
(60) Provisional application No. 61/920,701, filed on Dec. 24, 2013.
(60) Provisional application No. 61/933,383, filed on Jan. 30, 2014.

(51) Int. Cl. A61K 38/25; C12N 5/09; C07K 14/60; A61K 45/06; A61K 31/704

(52) U.S. Cl. CPC A61K 38/25; A61K 45/06; A61K 31/704; C07K 14/60; C12N 5/0693; C12N 2501/06; C12N 2501/36

(57) ABSTRACT

Embodiments of the present disclosure are directed to methods to treat a subject having cancer. In one embodiment, a method of treating a subject with cancer may comprise administering a therapeutically effective amount of at least one GHRH agonist peptide in combination with at least one anticancer agent. In another embodiment, a method of killing cancer cells may comprise contacting the cancer cells with a composition comprising at least one GHRH agonist peptide and at least one anticancer agent. In some embodiments, the method may be in vitro or in vivo. In an additional embodiment, a method of killing cancer stem cells may comprise contacting the cancer stem cells with a composition comprising at least one GHRH agonist peptide and at least one anticancer agent.

### CROSS-REFERENCE TO RELATED APPLICATIONS

This application claims priority to U.S. Provisional Application No. 61/920,701 filed on Dec. 24, 2013 and U.S. Provisional Application No. 61/933,383 filed on Jan. 30, 2014, both of which are incorporated herein by reference in its entirety.

### BRIEF SUMMARY OF THE INVENTION

Embodiments of the present disclosure are directed to methods of treating a subject having cancer. In one embodiment, a method of treating a subject with cancer may comprise administering a therapeutically effective amount of at least one GHRH agonist peptide. In further embodiments, the method may further comprise administering GHRH agonist peptide in combination with at least one anticancer agent. In such embodiments, the subject may have colorectal cancer, breast cancer, ovarian cancer, pancreatic cancer, head and neck cancer, bladder cancer, liver cancer, renal cancer, melanoma, gastrointestinal cancer, prostate cancer, small cell and non-small cell lung cancer, sarcomas, glioblastoma, T- and B-cell lymphoma, endometrial cancer, and cervical cancer.

In another embodiment, a method of killing cancer cells may comprise contacting the cancer cells with a composition comprising at least one GHRH agonist peptide in combination with at least one anticancer agent. In some embodiments, the method may be in vitro or in vivo.

In an additional embodiment, a method of killing cancer stem cells may comprise contacting the cancer stem cells with a composition comprising at least one GHRH agonist peptide in combination with at least one anticancer agent. The GHRH agonist peptide may be any GHRH peptide disclosed herein.

In the foregoing embodiments, the GHRH agonist peptide may comprise a peptide of formula I:

[R₁-A¹-A²-A³-Ala-Ile-A⁶-A⁷-A⁸-A⁹-A¹⁰-A¹¹-A¹²-A¹³-A¹⁴-A¹⁵-A¹⁶-A¹⁷-A¹⁸-Ala-A²⁰-A²¹-A²²-A²³-A²⁴-A²⁵-A²⁶-A²⁷-A²⁸-A²⁹-A³⁰]-R₂;
wherein,
R₁ is Ac, Tfa, alkyl, cycloalkyl, benzyl, phenyl, substituted alkyl, substituted phenyl, substituted phenyl ethyl, 2-carboxybenzamido, carboxypropyl, amino-(hydroxylphenyl)-alkyl, amino(imidazole-4-yl)-alkyl, hydroxyphenyl-alkyl, or is absent;
A¹ is Tyr, Dat, des-amino Tyr, D-Tyr, Met, Phe, D-Phe, pCl-Phe, Leu, His, D-His, or N-Me-Tyr;
A² is Ala, D-Ala, Abu, D-Arg, Aib, D-N-methyl Ala, or D-Abu;
A³ is Asp, D-Asp, Glu, or D-Glu;
A⁶ is Phe, or Fpa5;
A⁷ is Thr, Aib, Leu, Trp, β-Nal, or pX-Phe, where X is H, F, Cl, Br, NO₂, or Me;
A⁸ is D-Asn, Asn, Ala, Gln, Thr, N-Me-Ala, Aib, Leu, Trp, β-Nal, Ser, D-Ser, or pX-Phe, wherein X is H, F, Cl, Br, NO₂, or Me;
A⁹ is Ser, Ala, Aib, Leu, Trp, β-Nal, or pX-Phe, where X is H, F, Cl, Br, NO₂, or Me;
A¹⁰ is Tyr or D-Tyr;
A¹¹ is Arg, His, Gap, Gab, or Har;
A¹² is Orn, Arg, Lys, D-Lys, Lys(Me)₂, Gap, Gab, N-alkyl-Lys, or N-cycloalkyl-Lys;
A¹³ is Val or Ile;
A¹⁴ is Leu or D-Leu;
A¹⁵ is Abu, Gly, Leu, Asn, Gln, Aib, D-Ala, or Ala;
A¹⁶ is Gln, Ala, or Aib;
A¹⁷ is Leu or D-Leu;
A¹⁸ is Ser or Tyr;
A²⁰ is Arg, His, Gap, Gab, or Har;
A²¹ is Orn, Lys, D-Lys, Arg, D-Arg, Gap, Gab, or Lys(Me)₂;
A²² is Leu, Ala, Abu, Lys, or Orn;
A²³ is Leu, D-Leu, Ala, or Abu;
A²⁴ is Gln, His, Ala, or Aib;
A²⁵ is Asp, Glu, D-Glu, D-Asp, Ala, or Aib;
A²⁶ is Ile or Leu;
A²⁷ is Nle, Met, D-Met, Ala, Ile, Leu, Nva, or Val;
A²⁸ is Ser, Asn, or Asp;
A²⁹ is Arg, Har, Agm, D-Arg, D-Har, or D-Har;
A³⁰ is Arg, Agm, Ada, Amc, Aha, Apa, Har, D-Arg, D-Har, Gab, Gln, D-Gln, Gln-Gab, D-Gln-Gab, or is absent; and
R₂ is —NH₂, —NH—CH₃, —NH—CH₂—CH₃, —OH, —CH₂)ₚ—NH—C(NH₂)═NH, —NH(CH₂)ₚ—C(═O)—NH₂, —NHR₃, —N(R₃)₂, or —OR₃, where p is an integer from 1 to 15, and R₃ is any of C₁₋₁₂ alkyl, C₂₋₁₂ alkenyl, or C₂₋₁₂ alkynyl, and pharmaceutically acceptable salts thereof, and
wherein the agonist peptide is different from SEQ ID NO: 1 in at least one amino acid residue.

In some embodiments, the GHRH agonist peptide may comprise a peptide of formula II:

[R₁-A¹-A²-Asp-Ala-Ile-A⁶-Thr-A⁸-Ser-Tyr-A¹¹-A¹²-Val-Leu-A¹⁵-Gln-Leu-Ser-Ala-A²⁰-A²¹-A²²-Leu-Gln-Asp-Ala-Ile-Nle²⁷-A²⁸-A²⁹-A³⁰]-R₂,

wherein R₁ is Ac, Tfa, or is absent,
A¹ is Tyr, Dat, or N-Me-Tyr,
A² is Ala, D-Ala, Abu, or D-Abu,
A⁶ is Phe or Fpa5,
A⁸ is Asn, Ala, Gln, Thr, or N-Me-Ala,
A¹¹ is Arg, His, or Har,
A¹² is Orn, or Lys(Me)₂,
A¹⁵ is Abu or Ala,
A²⁰ is Arg, His, or Har,
A²¹ is Orn, or Lys(Me)₂,
A²² is Leu, or Orn,
A²⁸ is Ser, or Asp,
A²⁹ is Arg, Har, Agm, D-Arg, or D-Har,
A³⁰ is Arg, Agm, Ada, Amc, Aha, Apa, Har, D-Arg, D-Har, Gab, Gln, D-Gln,
Gln-Gab, D-Gln-Gab, or is absent,
R₂ is —NH₂, —NH—CH₃, —NH—CH₂—CH₃, —OH, —NHR₃, —N(R₃)₂, or —OR₃, wherein R₃ is any of C₁₋₁₂ alkyl, C₂₋₁₂ alkenyl, or C₂₋₁₂ alkynyl,
and pharmaceutically acceptable salts thereof, and wherein the agonist peptide is different from SEQ ID NO: 1 in at least one amino acid residue.

In some embodiments, the GHRH agonist peptide may be tesamorelin. In some embodiments, the GHRH agnist peptide may be a stapled GHRH agonist peptide. In some embodiments, the GHRH agonist peptide may be any one of formula II, IV or V.

### DESCRIPTION OF DRAWINGS

FIG. 1 discloses in vivo studies; (A) shows the effect of treatment with the GHRH agonist JI-34 (50 μg/kg/d or 1 μg/mouse/d) (J) and doxorubicin (DOX; D) (13 μmol/kg/wk) alone and in combination on the growth of U-87 MG, human GBM tumors xenotransplanted to nude mice. Numbers at labels represent the number of successfully implanted tumors. (B) shows final weights of necropsied tumor samples compared with the control. (C) shows numbers at the end of each curve show the tumor doubling times. *P<0.05 vs. control.

FIG. 2 shows the effect of single exposure (A) or repeated exposure (B) to GHRH agonist JI-34 (1 μM) (J) doxorubicin (100 nM DOX; D), and their combination on the proliferation of U-87 MG cells in vitro. *P<0.05 vs. control, ⁺P<0.05 vs. DOX. Panel C shows phasecontrast images of the U-87 MG cell cultures. Images are shown at 400× magnification with 20-megapixel resolution. Representative sections of the visual field were cropped and fitted. (Scale bars, 100 μm.).

FIG. 3 shows the effect of the combination treatment with the GHRH agonist JI-34+DOX on viability and apoptosis (A), calcein retention (B), and the expression of bFGF, TGFβ, and the tumor suppressor p53 (C), in vitro. *P<0.05 vs. control. DOX, doxorubicin; FGFb, fibroblast growth factor basic; TGFβ, transforming growth factor 0. *P<0.05 vs. control, ⁺P<0.05 vs. DOX.

FIG. 4 shows Western blot images (A) and integrated density values (IDVs) (B) for the expression of GHRH receptors and differentiation antigens in necropsied in vivo samples. GHRH-R, pituitary type growth hormone releasing hormone receptor; SV1, splice variant-1 of GHRH receptor; DOX or D, doxorubicin; J, JI-34; GFAP, glial fibrillary acidic protein. *P<0.05 vs. control.

FIG. 5 demonstrates GHRH agonist induced the differentiation of U-87 MG cells in reduced serum-containing medium in vitro. Cells were treated with GHRH agonist JI-34 (1 μM) or DMSO in 0.1% FBS-containing growth medium. Phase-contrast images of live cells were taken after 2 d and then cells were fixed and stained for the differentiation markers, GFAP and nestin. The GHRH agonist-treated cells possess a higher tendency toward outgrowth of projections; moreover, high levels of GFAP were detected in these cells, indicative of glial differentiation. The protein level of nestin did not change notably following the treatment with GHRH agonist. Nuclei were stained with DAPI. (Scale bars, 100 μm.).

### DETAILED DESCRIPTION

This invention is not limited to the particular processes, compositions, or methodologies described, as these may vary. The terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. Nothing herein is to be construed as an admission that the invention is not entitled to antedate such disclosure by virtue of prior invention.

As used herein and in the appended claims, the singular forms “a”, “an”, and “the” include plural reference unless the context clearly dictates otherwise. Thus, for example, reference to an “antioxidant” is a reference to one or more antioxidants and equivalents thereof known to those skilled in the art, and so forth.

As used herein, the term “about” means plus or minus 10% of the numerical value of the number with which it is being used. Therefore, about 50% means in the range of 45%-55%.

The term “animal,” “patient,” or “subject” as used herein includes, but is not limited to, humans and non-human vertebrates such as wild, domestic and farm animals. Preferably, the term refers to humans.

“Administering” when used in conjunction with a therapeutic means to administer a therapeutic to a patient whereby the therapeutic positively impacts the tissue to which it is targeted. The peptides/compounds described herein can be administered either alone or in combination (concurrently or serially) with other pharmaceuticals. For example, the peptides/compounds can be administered in combination with other anti-cancer or anti-neoplastic agents, or in combination with other cancer therapies other than chemotherapy, such as, for example, surgery or radiotherapy. In some embodiments, the peptides/compounds described herein can also be administered in combination with (i.e., as a combined formulation or as separate formulations) other therapeutics.

As used herein, the phrase “anticancer agents” refers to compounds or treatments that are effective in treating or preventing cancer including, without limitation, chemical agents, other immunotherapeutics, cancer vaccines, anti-angiogenic compounds, certain cytokines, certain hormones, gene therapy, radiotherapy, surgery, and dietary therapy.

A “therapeutically effective amount” or “effective amount” of a composition is a predetermined amount calculated to achieve the desired effect, i.e., to ameliorate, prevent or improve an unwanted condition, disease or symptom of a patient. The activity contemplated by the present methods may include both therapeutic and/or prophylactic treatment, as appropriate. The specific dose of the peptides/compounds or the peptides administered according to this invention to obtain therapeutic and/or prophylactic effects will, of course, be determined by the particular circumstances surrounding the case, including, for example, the peptides/compounds administered, the route of administration, and the condition being treated. The effective amount administered may be determined by a physician in the light of the relevant circumstances including the condition to be treated, the choice of peptides/compounds to be administered, and the chosen route of administration. A therapeutically effective amount of the peptide/compound of this invention is typically an amount such that when it is administered in a physiologically tolerable excipient composition, it is sufficient to achieve an effective systemic concentration or local concentration in the target tissue.

As used herein, the term “therapeutic” means an agent utilized to discourage, combat, ameliorate, prevent or improve an unwanted condition, disease or symptom of a patient.

By “pharmaceutically acceptable”, it is meant the carrier, diluent or excipient must be compatible with the other ingredients of the formulation and not deleterious to the recipient thereof.

As used herein, “analog” of polypeptides refers to an amino acid sequence that is altered by one or more amino acid residues. The analog may have “conservative” changes, wherein a substituted amino acid has similar structural or chemical properties (e.g., replacement of leucine with isoleucine). More rarely, an analog may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

By the term “modulate,” it is meant that any of the mentioned activities, are, e.g., increased, enhanced, increased, agonized (acts as an agonist), or promoted. Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

The terms “subject”, “patient” or “individual” are used interchangeably herein, and refers to a mammalian subject to be treated. In some embodiments, the patient is a human. In some cases, the methods can be used in experimental animals, in veterinary application, and in the development of animal models for disease, including, but not limited to, rodents including mice, rats, and hamsters; and primates. In some embodiments, the patient is a patient in need thereof.

As used herein, the phrase “in need thereof” means that the patient has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

“Treatment” is an intervention performed with the intention of preventing the development or altering the pathology or symptoms of a disorder. Accordingly, “treatment” can refer to therapeutic treatment or prophylactic or preventative measures. In some embodiments, the treatment is for therapeutic treatment. In some embodiments, the treatment is for prophylactic or preventative treatment. Those in need of treatment can include those already with the disorder as well as those in which the disorder is to be prevented.

As used herein, “ameliorated” or “treatment” refers to a symptom which is approaches a normalized value (for example a value obtained in a healthy patient or individual), e.g., is less than 50% different from a normalized value, is less than about 25% different from a normalized value, is less than 10% different from a normalized value, or is not significantly different from a normalized value as determined using routine statistical tests.

“Agonist of GHRH” means a compound or peptide other than GHRH which has the function of binding to and stimulating GHRH receptors, resulting in the release of growth hormone, or another physiological, endocrine or cellular response specific for GHRH. In some embodiments, a GHRH agonist may activate GHRH receptor and may not result in the release of growth hormone. A GHRH agonist may comprise a full length GHRH sequence in which certain modifications have been made, e.g., amino acid residues have been substituted, side groups have been added. The amino acid sequence of hGHRH (1-30), starting at the N-terminal part is: Tyr¹-Ala²-Asp³-Ala⁴-Ile⁵-Phe⁶-Thr⁷-Asn⁸-Ser⁹-Tyr¹⁰-Arg¹¹-Lys¹²-Val¹³-Leu¹⁴-Gly¹⁵-Gln¹⁶-Leu¹⁷-Ser¹⁸-Ala¹⁹-Arg²⁰-Lys²¹-Leu²²-Leu²³-Gln²⁴-Asp²⁵-Ile²⁶-Met²⁷-Ser²⁸-Arg²⁹-Gln³⁰ (SEQ ID NO: 1). A GHRH agonist may comprise a GHRH sequence to which amino acid deletions, insertions, and/or substitutions have been made. A GHRH agonist may also be a fragment or modified fragment of GHRH having the capability to bind to the GHRH receptor and stimulate release of growth hormone. Such GHRH agonists include, GHRH(1-29), GHRH(1-30) and GHRH(1-44) peptide fragments. The biological activity of GHRH is understood to reside in the N-terminal amino acid sequences of the hormone. Thus, fragments or modified fragments between amino acid residues 1 and 30, between amino acid residues 1 and 29, or between amino acid residues 1 and 44 are expected to be useful.

As used herein, the phrase “preventing cancer” refers to prevention of cancer occurrence. In certain instances, the preventative treatment reduces the recurrence of the cancer. In other instances, preventative treatment decreases the risk of a patient from developing a cancer, or inhibits progression of a pre-cancerous state (e.g. a colon polyp) to actual malignancy.

As used herein, the phrase “treating cancer” refers to inhibition of cancer cell replication, apoptosis, inhibition of cancer spread (metastasis), inhibition of tumor growth, reduction of cancer cell number or tumor growth, decrease in the malignant grade of a cancer (e.g., increased differentiation), or improved cancer-related symptoms.

Disclosed herein are a 1 series of synthetic peptide analogs of hGHRH(1-29) or hGHRH(1-30) that may be used to treat cancer. The synthetic peptides of this invention exhibit high activities in stimulating the release of pituitary growth hormone (GH) in animals, including humans. They also show extremely high binding capacity to the hGHRH receptor. These synthetic hGHRH analogs also retain their physiological activity in solution for an extended period of time and resist enzymatic degradation in the body. The stronger GH releasing potencies of the new analogs in vivo, as compared to previously described ones, results from combination of replacements in hGHRH(1-29) or hGHRH(1-30) and from resistance to in vivo degradation. Without in any way limiting the invention or its scope, applicants wish to express their understanding that the retention of activity in vitro and resistance to in vivo degradation are due to multiple substitutions in the molecule: incorporation of N-Me-Tyr or des-amino-Tyr (Dat) in position 1 which protect peptides from the degradation at the N-terminus; incorporation of agmatine (Agm) or —NH—CH₃ or —NH—CH₂—CH₃ at position 29 or extension of the C-terminus with an omega-amino acid which protects peptides from degradation at the C-terminus; and also the replacements of both lysines in the synthetic peptides with ornithine (Orn), which is a poor substrate for trypsin-like enzymes; Gly at residue 15 by Abu. To increase chemical stability, Asn at position 8 is replaced by Gln, Thr, or Ala. And Met in position 27 is replaced by norleucine (Nle). Replacement of other residues in the peptides and the combination of these replacements also are found to promote biological activity.

For example, an agonist of GHRH can include one or more features that protect it against degradation by biological, chemical, and/or other processes. For example, such features can protect the GHRH agonist peptide from proteolytic enzymes in the wound milieu (fluids), e.g., from proteases secreted by neutrophils. Such proteolytic enzymes can inactivate (e.g., degrade or split) unprotected peptides such as unprotected GHRH. Such protective features can include, for example, the replacement of certain amino acids (residues) in the native peptide sequence of GHRH with other different amino acids (residues). In some embodiments, replacement of Arg in position 29 by Agm (agmatine, 4-guanidino-butylamine) may provide resistance to enzymatic degradation of the peptide at the C-terminus. In some embodiments, replacement of Tyr in position 1 by des-aminotyrosine (Dat) may result in peptides with increased biological activities as a result of the resistance to N-terminal enzymatic degradation. Similarly, substitutions of hydrophobic groups at the C-terminal of peptides can result in significant increase in specific activity of the peptides.

In some embodiments, the GHRH agonist peptide is represented by the formula I:

[R¹-A¹-A²-A³-Ala-Ile-A⁶-A⁷-A⁸-A⁹-A¹⁰-A¹¹-A¹²-A¹³-A¹⁴-A¹⁵-A¹⁶-A¹⁷-A¹⁸-Ala-A²⁰-A²¹-A²²-A²³-A²⁴-A²⁵-A²⁶-A²⁷-A²⁸-A²⁹-A³⁰]-R²;
In some embodiments, R¹ is Ac, Tfa, alkyl, cycloalkyl, benzyl, phenyl, substituted alkyl, substituted phenyl, substituted phenyl ethyl, 2-carboxybenzamido, carboxypropyl, amino-(hydroxylphenyl)-alkyl, amino(imidazole-4-yl)-alkyl, hydroxyphenyl-alkyl, or is absent;
In some embodiments, A¹ is Tyr, Dat, des-amino Tyr, D-Tyr, Met, Phe, D-Phe, pCl-Phe, Leu, His, D-His, or N-Me-Tyr;
In some embodiments, A² is Ala, D-Ala, Abu, D-Arg, Aib, D-N-methyl Ala, or D-Abu;
In some embodiments, A³ is Asp, D-Asp, Glu, or D-Glu;
In some embodiments, A⁶ is Phe, or Fpa5;
In some embodiments, A⁷ is Thr, Aib, Leu, Trp, β-Nal, or pX-Phe, where X is H, F, Cl, Br, NO₂, or Me;
In some embodiments, A⁸ is D-Asn, Asn, Ala, Gln, Thr, N-Me-Ala, Aib, Leu, Trp, β-Nal, Ser, D-Ser, or pX-Phe, wherein X is H, F, Cl, Br, NO2, or Me;
In some embodiments, A⁹ is Ser, Ala, Aib, Leu, Trp, β-Nal, or pX-Phe, where X is H, F, Cl, Br, NO₂, or Me;
In some embodiments, A¹⁰ is Tyr or D-Tyr;
In some embodiments, A¹¹ is Arg, His, Gap, Gab, or Har;
In some embodiments, A¹² is Orn, Arg, Lys, D-Lys, Lys(Me)₂, Gap, Gab, N-alkyl-Lys, or N-cycloalkyl-Lys;
In some embodiments, A¹³ is Val or Ile;
In some embodiments, A¹⁴ is Leu or D-Leu;
In some embodiments, A¹⁵ is Abu, Gly, Leu, Asn, Gln, Aib, D-Ala, or Ala;
In some embodiments, A¹⁶ is Gln, Ala, or Aib;
In some embodiments, A¹⁷ is Leu or D-Leu;
In some embodiments, A¹⁸ is Ser or Tyr;
In some embodiments, A²⁰ is Arg, His, Gap, Gab, or Har;
In some embodiments, A²¹ is Orn, Lys, D-Lys, Arg, D-Arg, Gap, Gab, or Lys(Me)₂;
In some embodiments, A²² is Leu, Ala, Abu, Lys, or Orn;
In some embodiments, A²³ is Leu, D-Leu, Ala, or Abu;
In some embodiments, A²⁴ is Gln, His, Ala, or Aib;
In some embodiments, A²⁵ is Asp, Glu, D-Glu, D-Asp, Ala, or Aib;
In some embodiments, A²⁶ is Ile or Leu;
In some embodiments, A²⁷ is Nle, Met, D-Met, Ala, Ile, Leu, Nva, or Val;
In some embodiments, A²⁸ is Ser, Asn, or Asp;
In some embodiments, A²⁹ is Arg, Har, Agm, D-Arg, D-Har, or D-Har;
In some embodiments, A³⁰ is Arg, Agm, Ada, Amc, Aha, Apa, Har, D-Arg, D-Har, Gab, Gln, D-Gln, Gln-Gab, D-Gln-Gab, or is absent;
In some embodiments, R² is —NH₂, —NH—CH₃, —NH—CH₂—CH₃, —OH, —CH₂)p-NH—C(NH₂)═NH, —NH(CH₂)p-C(═O)—NH₂, —NHR3, —N(R³)₂, or —OR³, where p is an integer from 1 to 15, and R3 is any of C₁₋₁₂ alkyl, C₂₋₁₂ alkenyl, or C₂₋₁₂ alkynyl;
In some embodiments, the peptides are in the form of pharmaceutically acceptable salts;
In some embodiments, the agonist peptide is different from SEQ ID NO: 1 in at least one amino acid residue; and
In some embodiments, if the A²⁹ is Agm, then A³⁰ and R² are absent, and A¹ is N-Me-Tyr.

The nomenclature used to define the amino acid residues and synthetic peptides is according to the IUPAC-IUB Commission on Biochemical Nomenclature (European J. Biochem., 1984, 138, 9-37). The naturally occurring amino acids found in proteins are depicted by the following three letter codes: Gly, Ala, Val, Leu, Ile, Ser, Thr, Lys, Arg, Asp, Asn, Glu, Gln, Cys, Met Phe, Tyr, Pro, Trp and His.
Other abbreviations used are:
Aah alpha-amino-hexanoic acid
Aap alpha-amno-pentanoic acid
Abu alpha-aminobutyric acid
Ac acetyl
AcOH acetic acid
Ac₂O acetic anhydride
Ada 12-aminododecanoyl
Agm agmatine
Aha 6-aminohexanoyl
AM aminomethyl
Amc 8-Aminocaprylyl
Apa 5-Aminopentanoyl
Aib alpha-aminoisobutyroyl
Boc tert-butyloxycarbonyl
Bom benzyloxymethyl
2BrZ 2-bromo-benzyloxycarbonyl
Bul tertiary butyl (ester)
Bzl benzyl cHx cyclohexyl
2ClZ 2-chloro-benzyloxycarbonyl
2ClTrt 2-chlorotrityl
Cpa para-chlorophenylalanine
Dat des-amino-tyrosine
DCM dichloromethane
DIC N,N′-diisopropylcarbodiimide
DIEA diisopropylethylamine
DMF dimethylformamide
Et ethyl
Fm fluorenylmethyl
Fmoc fluorenylmethoxycarbonyl
For formyl
Fpa mono- or poly-fluorinated Phe (fluorine substitution on the aromatic ring)
Fpa5 pentafluoro-Phe
Gab 4-guanidino-2-amino butyric acid
Gap 3-guanidino-2-aminopropionic acid
GH growth hormone
GHRH GH releasing hormone
Har homoarginine
HBTU 2-(1H-benzotriazol-1-yl)-1,1,3,3-tetramethyluronium hexaflourophosphate
hGHRH human GHRH
HOBt 1-hydroxybenzotriazole
HPLC high performance liquid chromatography
Ibu isobutyryl
MBHA para-methylbenzhydrylamine
Me methyl
MeOH methanol
MeCN acetonitrile
Mmt 4-methoxytrityl
Mtr 4-methoxy-2,3,6-trimethylbenzenesulphonyl
N-Me-Ala N-methyl-Ala
N-Me-Tyr N-methyl-Tyr
β-Nal beta-naphthylalanine
Nle norleucine
NMM N-methylmorpholine
Nva Norvaline
Oaa omega-amino acid
Orn ornithine
PAM phenylacetamidomethyl
Pbf 2,2,4,6,7-pentamethyl-dihydrobenzofurane-5-sulfonyl
Ph phenyl
PS polystyrene
rGHRH ratGHRH
RP-HPLC reversed phase HPLC
SPA para-sulfonyl-phenoxyacetyl
fBu tertiary butyl (ether)
TFA trifluoroacetic acid
Tfa trifluoroacetyl
Tos para-toluenesulfonyl
Trt trityl (triphenylmethyl)
Z benzyloxycarbonyl

The amino acid sequences of the synthetic peptides are numbered in correspondence with the amino acid residues in wild-type hGHRH(1-30) (SEQ ID NO: 1); thus, for example, the synthetic peptide P-20103 may be represented in an abbreviated form as below:

[N-Me-Tyr¹,Fpa5⁶,Gln⁸,Orn¹²,Abu¹⁵,Orn²¹,Nle²⁷,Asp²⁸,Agm²⁹]hGHRH(1-29).

The residue N-Me-Tyr^{a} represents substitution at position 1 of wild-type hGHRH(1-30)NH₂ (SEQ ID NO: 1) in place of Tyr; Fpa5⁶ represents substitution position 6 in place of Phe; Gln⁸ represents substitution at position 8 in place of Asn, and so on. Further, the amino acid residues at the positions which are not recited in the above abbreviated form (positions A², A³, A⁴, A⁵, A⁷, A⁹, A¹⁰, A¹¹, A¹³, A¹⁴, A¹⁶, A¹⁷, A¹⁸, A¹⁹, A²⁰, A²², A²³, A²⁴, A²⁵, A²⁶) correspond to the amino acid residues of the wild-type hGHRH(1-30)NH₂ (SEQ ID NO: 1). Thus, the abbreviated form of a synthetic peptide represent different substitutions when compared to the wild-type hGHRH(1-30)NH₂ (SEQ ID NO: 1). Further, in some embodiments, the synthetic peptides described herein may be 30 amino acids in length, as represented by hGHRH(1-30). In some embodiments, the synthetic peptides may be 29 amino acids in length, as represented by hGHRH(1-29)NH₂. The convention under which the N-terminal of a peptide is placed to the left, and the C-terminal to the right is also followed herein.

In some embodiments, the GHRH agonist peptide is represented by the formula II:

[R¹-A¹-A²-Asp-Ala-Ile-A⁶-Thr-A⁸-Ser-Tyr-A¹¹-A¹²-Val-Leu-A¹⁵-Gln-Leu-Ser-Ala-A²⁰-A²¹-A²²-Leu-Gln-Asp-Ile-Nle²⁷-A²⁸-A²⁹-A³⁰]-R².
In some embodiments, R¹ is Ac, Tfa, or is absent;
In some embodiments, A¹ is Tyr, Dat, or N-Me-Tyr;
In some embodiments, A² is Ala, D-Ala, Abu, or D-Abu;
In some embodiments, A⁶ is Phe or Fpa5;
In some embodiments, A⁸ is Asn, Ala, Gln, Thr, or N-Me-Ala;
In some embodiments, A¹¹ is Arg, His, or Har;
In some embodiments, A¹² is Orn, or Lys(Me)2;
In some embodiments, A¹⁵ is Abu or Ala;
In some embodiments, A²⁰ is Arg, His, or Har;
In some embodiments, A²¹ is Orn, or Lys(Me)2;
In some embodiments, A²² is Leu, or Orn;
In some embodiments, A²⁸ is Ser, or Asp;
In some embodiments, A²⁹ is Arg, Har, Agm, D-Arg, or D-Har;
In some embodiments, A³⁰ is Arg, Agm, Ada, Amc, Aha, Apa, Har, D-Arg, D-Har, Gab, Gln, D-Gln, Gln-Gab, D-Gln-Gab, or is absent;
In some embodiments, R² is —NH₂, —NH—CH₃, —NH—CH₂—CH₃, —OH, —NHR₃, —N(R³)₂, or —OR³, wherein R³ is any of C₁₋₁₂ alkyl, C₂₋₁₂ alkenyl, or C₂₋₁₂ alkynyl, and pharmaceutically acceptable salts thereof;
In some embodiments, the agonist peptide is different from SEQ ID NO: 1 in at least one amino acid residue; and
In some embodiments, if the A²⁹ is Agm, then A³⁰ and R² are absent, and A¹ is N-Me-Tyr.

Suitable synthetic hGHRH agonist peptides in abbreviated form that are covered by formula I and II are disclosed in Table 1.

TABLE 1
P-20103 [N-Me-Tyr¹, Fpa5⁶, Gln⁸, Orn¹², Abu¹⁵, Orn²¹, Nle²⁷, Asp²⁸, Agm²⁹]
hGHRH(1-29)NH₂(SEQ ID NO: 2);
P-20105 [N-Me-Tyr¹, D-Ala², Fpa5⁶, Gln⁸, Orn¹², Abu¹⁵, Orn²¹, Nle²⁷, Asp²⁸, Agm²⁹]
hGHRH(1-29)NH₂;
P-20107 [N-Me-Tyr¹, Fpa5⁶, Ala⁸, Orn¹², Abu¹⁵, Orn²¹, Nle²⁷, Asp²⁸, Agm²⁹]
hGHRH(1-29)NH₂(SEQ ID NO: 3);
P-20109 [N-Me-Tyr¹, D-Ala², Fpa5⁶, Ala⁸, Orn¹², Abu¹⁵, Orn²¹, Nle²⁷, Asp²⁸, Agm²⁹]
hGHRH(1-29)NH₂;
P-20110 [N-Me-Tyr¹, D-Ala², Fpa5⁶, Orn¹², Abu¹⁵, Orn²¹, Nle²⁷, Agm²⁹] hGHRH(1-29)
NH₂;
P-20111 [N-Me-Tyr¹, D-Ala², Fpa5⁶,Thr⁸, Orn¹², Abu¹⁵, Orn²¹, Nle²⁷, Asp²⁸, Agm²⁹]
hGHRH(1-29)NH₂;
P-20113 [N-Me-Tyr¹, Fpa5⁶, Orn¹², Abu¹⁵, Orn²¹, Nle²⁷, Asp²⁸, Agm²⁹]hGHRH (1-29)
NH₂(SEQ ID NO: 4);
P-20115 [N-Me-Tyr¹, Fpa5⁶, Thr⁸, Orn¹², Abu¹⁵, Orn²¹, Nle²⁷, Asp²⁸, Agm²⁹] hGHRH
(1-29)NH₂(SEQ ID NO: 5);
P-20117 [N-Me-Tyr¹, D-Ala², Fpa⁵⁶, Gln⁸, Orn¹², Abu¹⁵, Orn²¹, Nle²⁷, Agm²⁹] hGHRH
(1-29) NH₂;
P-20350 [Dat¹, D-Ala², Gln⁸, Orn¹², Abu¹⁵, Orn²¹, Nle²⁷, Asp²⁸, Agm²⁹] hGHRH(1-29)
NH₂;
P-20351 [Ac-N-Me-Tyr¹, D-Ala², Gln⁸, Orn¹², Abu¹⁵, Orn²¹, Nle²⁷, Asp²⁸, Agm²⁹]
hGHRH(1-29)NH₂;
P-20356 [N-Me-Tyr¹, Gln⁸, Orn¹², Abu¹⁵, Orn²¹, Nle²⁷, Asp²⁸, Agm²⁹]hGHRH(1-29)
NH₂ (SEQ ID NO: 6);
P-20357 [Dat¹, D-Ala², N-Me-Ala⁸, Orn¹², Abu¹⁵, Orn²¹, Nle²⁷, Asp²⁸, Agm²⁹] hGHRH
(1-29)NH₂;
P-20358 [N-Me-Tyr¹, D-Ala², N-Me-Ala⁸, Orn¹², Abu¹⁵, Orn²¹, Nle²⁷, Asp²⁸, Agm²⁹]
hGHRH(1-29)NH₂;
P-20359 [N-Me-Tyr¹, D-Ala², Fpa5⁶, Orn¹², Abu¹⁵, Orn²¹, Nle²⁷, Asp²⁸, Agm²⁹] hGHRH
(1-29)NH₂;
P-20360 [N-Me-Tyr¹, D-Ala², Thr⁸, Orn¹², Abu¹⁵, Orn²¹, Nle²⁷, Asp²⁸, Agm²⁹] hGHRH
(1-29)NH₂;
P-20361 [N-Me-Tyr¹, D-Ala², Gln⁸, Orn¹², Abu¹⁵, Orn²¹, Nle²⁷, Asp²⁸, Agm²⁹] hGHRH
(1-29)NH₂;
P-20367 [N-Me-Tyr¹, D-Ala², Orn¹², Abu¹⁵, Orn²¹, Nle²⁷, Asp²⁸, Agm²⁹] hGHRH (1-
29)NH₂;
P-20370 [N-Me-Tyr¹, Orn¹², Abu¹⁵, Orn²¹, Nle²⁷, Asp²⁸, Agm²⁹]hGHRH(1-29)NH₂
(SEQ ID NO: 7);
P-20371 [N-Me-Tyr¹, Thr⁸, Orn¹², Abu¹⁵, Orn²¹, Nle²⁷, Asp²⁸, Agm²⁹]hGHRH (1-29)
NH₂ (SEQ ID NO: 8);
P-20372 [N-Me-Tyr¹, Ala⁸, Orn¹², Abu¹⁵, Orn²¹, Nle²⁷, Asp²⁸, Agm²⁹]hGHRH (1-29)
NH₂ (SEQ ID NO: 9);
P-20373 [N-Me-Tyr¹, Gln⁸, Orn¹², Abu¹⁵, Orn²¹, Nle²⁷, Agm²⁹]hGHRH(1-29)NH₂
(SEQ ID NO: 10);
P-20374 [N-Me-Tyr¹, Orn¹², Abu¹⁵, Orn²¹, Nle²⁷, Agm²⁹]hGHRH(1-29)NH₂ (SEQ
ID NO: 11);
P-20375 [N-Me-Tyr¹, Thr⁸, Orn¹², Abu¹⁵, Orn²¹, Nle²⁷, Agm²⁹]hGHRH(1-29)NH₂
(SEQ ID NO: 12);
P-20376 [N-Me-Tyr¹, Ala⁸, Orn¹², Abu¹⁵, Orn²¹, Nle²⁷, Agm²⁹]hGHRH(1-29)NH₂
(SEQ ID NO: 13);
P-21300 [Dat¹, D-Ala², Orn¹², Abu¹⁵, Orn²¹, Nle²⁷, Asp²⁸, Amc³⁰]hGHRH(1-30)NH₂;
P-21301 [N-Me-Tyr¹, Orn¹², Abu¹⁵, Orn²¹, Nle²⁷, Asp²⁸, Amc³⁰]hGHRH(1-30)NH₂
(SEQ ID NO: 14);
P-21303 [N-Me-Tyr¹, D-Ala², Orn¹², Abu¹⁵, Orn²¹, Nle²⁷, Asp²⁸, Amc³⁰]hGHRH(1-
30)NH₂;
P-21304 [Dat¹, D-Ala², Gln⁸, Orn¹², Abu¹⁵, Orn²¹, Nle²⁷, Asp²⁸, Amc³⁰]hGHRH(1-
30)NH₂;
P-21305 [N-Me-Tyr¹, D-Ala², Gln⁸, Orn¹², Abu¹⁵, Orn²¹, Nle²⁷, Asp²⁸, Amc³⁰]
hGHRH(1-30)NH₂;
P-21306 [Dat¹, D-Ala², Thr⁸, Orn¹², Abu¹⁵, Orn²¹, Nle²⁷, Asp²⁸, Amc³⁰]hGHRH(1-
30)NH₂;
P-21307 [N-Me-Tyr¹, D-Ala², Thr⁸, Orn¹², Abu¹⁵, Orn²¹, Nle²⁷, Asp²⁸, Amc³⁰]
hGHRH(1-30)NH₂;
P-21308 [Dat¹, D-Ala², Ala⁸, Orn¹², Abu¹⁵, Orn²¹, Nle²⁷, Asp²⁸, Amc³⁰]hGHRH(1-
30)NH₂;
P-21309 [N-Me-Tyr¹, D-Ala², Orn¹², Ala⁸, Abu¹⁵, Orn²¹, Nle²⁷, Asp²⁸, Amc³⁰]hGH-
RH(1-30)NH₂;
P-21310 [Dat¹, D-Ala², His¹¹, Orn¹², Abu¹⁵, His²⁰, Orn²¹, Nle²⁷, Asp²⁸, Amc³⁰]
hGHRH(1-30)NH₂;
P-21311 [N-Me-Tyr¹, D-Ala², His¹¹, Orn¹², Abu¹⁵, His²⁰, Orn²¹, Nle²⁷, Asp²⁸,
Amc³⁰]hGHRH(1-30)NH₂;
P-22325 [N-Me-Tyr¹, Gln⁸, Orn¹², Abu¹⁵, Orn²¹, Nle²⁷, Asp²⁸, Apa³⁰]hGHRH(1-
30)NH₂ (SEQ ID NO: 15);
P-22326 [N-Me-Tyr¹, D-Ala², Gln⁸, Orn¹², Abu¹⁵, Orn²¹, Nle²⁷, Asp²⁸, Apa³⁰]
hGHRH(1-30)NH₂;
P-22327 [Dat¹, Gln⁸, Orn¹², Abu¹⁵, Orn²¹, Nle²⁷, Asp²⁸, Apa³⁰]hGHRH(1-30)NH₂
(SEQ ID NO: 16);
P-22328 [Ac-Tyr¹, D-Ala², Gln⁸, Orn¹², Abu¹⁵, Orn²¹, Nle²⁷, Asp²⁸, Apa³⁰]
hGHRH(1-30)NH₂;
P-22329 [N-Me-Tyr¹, D-Ala², Orn¹², Abu¹⁵, Orn²¹, Nle²⁷, Apa³⁰]hGHRH(1-30)NH₂;
P-22330 [N-Me-Tyr¹, D-Ala², Thr⁸, Orn¹², Abu¹⁵, Orn²¹, Nle²⁷, Asp²⁸, Apa³⁰]
hGHRH(1-30)NH₂;
P-22331 [N-Me-Tyr¹, D-Ala², Ala⁸, Orn¹², Abu¹⁵, Orn²¹, Nle²⁷, Asp²⁸, Apa³⁰]
hGHRH(1-30)NH₂;
P-22332 [N-Me-Tyr¹, Orn¹², Abu¹⁵, Orn²¹, Nle²⁷, Asp²⁸, Apa³⁰]hGHRH(1-30)NH₂
(SEQ ID NO: 17);
P-22334 [N-Me-Tyr¹, Orn¹², Abu¹⁵, Orn²¹, Nle²⁷, Apa³⁰]hGHRH(1-30)NH₂ (SEQ ID
NO: 18);
P-22335 [N-Me-Tyr¹, Thr⁸, Orn¹², Abu¹⁵, Orn²¹, Nle²⁷, Asp²⁸, Apa³⁰]hGHRH(1-
30)NH₂ (SEQ ID NO: 19);
P-22336 [N-Me-Tyr¹, Ala⁸, Orn¹², Abu¹⁵, Orn²¹, Nle²⁷, Asp²⁸, Apa³⁰]hGHRH(1-
30)NH₂ (SEQ ID NO: 20);
P-22337 [N-Me-Tyr¹, D-Ala², Cpa⁶, Gln⁸, Orn¹², Abu¹⁵, Orn²¹, Nle²⁷, Asp²⁸, Apa³⁰]
hGHRH(1-30)NH₂;
P-23250 [Dat¹, D-Ala², Gln⁸, Orn¹², Abu¹⁵, Orn²¹, Nle²⁷, Asp²⁸, Har³⁰]hGHRH(1-
30)NH₂;
P-23251 [Dat¹, Gln⁸, Orn¹², Abu¹⁵, Orn²¹, Nle²⁷, Asp²⁸, Har³⁰]hGHRH(1-30)NH₂ (SEQ
ID NO: 21);
P-23252 [Dat¹, Orn¹², Abu¹⁵, Orn²¹, Nle²⁷, Asp²⁸, Har³⁰]hGHRH(1-30)NH₂;
P-23253 [Dat¹, Orn¹², Abu¹⁵, Orn²¹, Nle²⁷, Asp²⁸, Har³⁰]hGHRH(1-30)NH₂ (SEQ ID NO: 22);
P-23254 [Dat¹, D-Ala², Thr⁸, Orn¹², Abu¹⁵, Orn²¹, Nle²⁷, Asp²⁸, Har³⁰]hGHRH(1-30)NH₂;
P-23255 [Dat¹, Thr⁸, Orn¹², Abu15, Orn²¹, Nle²⁷, Asp²⁸, Har³⁰]hGHRH(1-30)NH₂
(SEQ ID NO: 23);
P-23256 [Dat¹, D-Ala², Ala⁸, Orn¹², Abu¹⁵, Orn²¹, Nle²⁷, Asp²⁸, Har³⁰]hGHRH(1-30)NH₂;
P-23257 [Dat¹, Ala⁸, Orn¹², Abu15, Orn²¹, Nle²⁷, Asp²⁸, Har³⁰(SEQ ID NO:
24);
P-23258 [N-Me-Tyr¹, D-Ala², Gln⁸, Orn¹², Abu¹⁵, Orn²¹, Nle²⁷, Asp²⁸, Har³⁰]hGHRH(1-
30)NH₂;
P-23259 [N-Me-Tyr¹, Gln⁸, Orn¹², Abu¹⁵, Orn²¹, Nle²⁷, Asp²⁸, Har³⁰]hGHRH(1-30)NH₂
(SEQ ID NO: 25);
P-23260 [N-Me-Tyr¹, D-Ala², Orn¹², Abu¹⁵, Orn²¹, Nle²⁷, Asp²⁸, Har³⁰]hGHRH(1-30)NH₂;
P-23261 [N-Me-Tyr¹, Orn¹², Abu¹⁵, Orn²¹, Nle²⁷, Asp²⁸, Har³⁰]hGHRH(1-30)NH₂ (SEQ ID NO:
26);
P-23262 [N-Me-Tyr¹, D-Ala², Thr⁸, Orn¹², Abu¹⁵, Orn²¹, Nle²⁷, Asp²⁸, Har³⁰]hGHRH(1-
30)NH₂;
P-23263 [N-Me-Tyr¹, Thr⁸, Orn¹², Abu¹⁵, Orn²¹, Nle²⁷, Asp²⁸, Har³⁰]hGHRH(1-30)NH₂ (SEQ
ID NO: 27);
P-23264 [N-Me-Tyr¹, D-Ala², Ala⁸, Orn¹², Abu¹⁵, Orn²¹, Nle²⁷, Asp²⁸, Har³⁰]hGHRH(1-
30)NH₂;
P-23265 [N-Me-Tyr¹, Ala⁸, Orn¹², AbU¹⁵, Orn²¹, Nle²⁷, Asp²⁸, Har³⁰]hGHRH(1-30)NH₂ (SEQ
ID NO: 28);
P-24340 [N-Me-Tyr¹, Gln⁸, Orn¹², Abu¹⁵, Orn²¹, Nle²⁷, Asp²⁸, Aha³⁰]hGHRH(1-30) NH₂
(SEQ ID NO: 29);
P-24341 [N-Me-Tyr¹, D-Ala², Gln⁸, Orn¹², Abu¹⁵, Orn²¹, Nle²⁷, Asp²⁸, Aha³⁰]hGHRH(1-
30)NH₂;
P-24342 [Dat¹, Gln⁸, Orn¹², Abu¹⁵, Orn²¹, Nle²⁷, Asp²⁸, Aha³⁰]hGHRH(1-30) NH₂ (SEQ ID
NO: 30);
P-24344 [Dat¹-D-Ala², Gln⁸, Orn¹², Abu¹⁵, Orn²¹, Nle²⁷, Asp²⁸, Aha³⁰]hGHRH(1-
30)NH₂;
P-24345 [N-Me-Tyr¹, D-Ala², Orn¹², Abu¹⁵, Orn²¹, Nle²⁷, Asp²⁸, Aha³⁰]hGHRH(1-
30)NH₂;
P-24346 [N-Me-Tyr¹, D-Ala², Gln⁸, Orn¹², Abu¹⁵, Orn²¹, Nle²⁷, Aha³⁰]hGHRH(1-
30)NH₂;
P-24347 [N-Me-Tyr¹, D-Ala², Thr⁸, Orn¹², Abu¹⁵, Orn²¹, Nle²⁷, Asp²⁸, Aha³⁰]
hGHRH(1-30)NH₂;
P-24348 [N-Me-Tyr¹, D-Ala², Ala⁸, Orn¹², Abu¹⁵, Orn²¹, Nle²⁷, Asp²⁸, Aha³⁰]
hGHRH(1-30)NH₂;
P-25501 [N-Me-Tyr¹, D-Ala², Orn¹², Abu¹⁵, Orn²¹, Nle²⁷, Gab³⁰]hGHRH(1-30)NH₂;
P-25502 [Dat¹, D-Ala², Fpa5⁶, Orn¹², Abu¹⁵, Orn²¹, Nle²⁷, Gab³⁰]hGHRH(1-30)NH₂;
P-25503 [N-Me-Tyr¹, Abu², Orn¹², Abu¹⁵, Orn²¹, Nle²⁷, Gab³⁰]hGHRH(1-30)NH₂
(SEQ ID NO: 31);
P-25504 [Dat¹, D-Abu², Orn¹², Abu¹⁵, Orn²¹, Nle²⁷, Gab³⁰]hGHRH(1-30)NH₂;
P-25506 [N-Me-Tyr¹, D-Abu², Orn¹², Abu¹⁵, Orn²¹, Nle²⁷, Gab³⁰]hGHRH(1-30)NH₂;
P-25508 [Tfa-Tyr¹, D-Ala², Orn¹², Abu¹⁵, Orn²¹, Nle²⁷, Gab³⁰]hGHRH(1-30)NH₂;
P-25516 [N-Me-Tyr¹, D-Ala², Orn¹², Abu¹⁵, Orn²¹, Nle²⁷, Asp²⁸, Gab³⁰]hGHRH(1-
30)NH₂;
P-26802 [Dat¹, D-Ala², Thr⁸, His¹¹, Orn¹², Abu¹⁵, His²⁰, Orn²¹, Nle²⁷, Ada³⁰]
hGHRH(1-30)NH₂;
P-26803 [N-Me-Tyr¹, D-Ala², Gln⁸, His¹¹, Orn¹², Abu¹⁵, His²⁰, Orn²¹, Nle²⁷, Ada³⁰]
hGHRH(1-30)NH-CH₃;
P-26804 [N-Me-Tyr¹, D-Ala², Gln⁸, Orn¹², Abu¹⁵, Orn²¹, Nle²⁷, Ada³⁰]hGHRH(1-
30)NH₂;
P-27400 [Dat¹, D-Ala², Orn¹², Abu¹⁵, Orn²¹, Nle²⁷, Asp²⁸]hGHRH(1-29)NH-CH₃;
P-27401 [Dat¹, D-Ala², Gln⁸, Orn¹², Abu¹⁵, Orn²¹, Nle²⁷, Asp²⁸, D-Arg²⁹]hGHRH(1-
29)NH-CH₃;
P-27403 [N-Me-Tyr¹, D-Ala², Gln⁸, Orn¹², Abu¹⁵, Orn²¹, Nle²⁷, Asp²⁸]hGHRH(1-
29)NH-CH₃;
P-27404 [N-Me-Tyr¹, D-Ala², Fpa5⁶, Orn¹², Abu¹⁵, Orn²¹, Nle²⁷, Asp²⁸]hGHRH (1-
29)NH-CH₃;
P-27405 [N-Me-Tyr¹, Gln⁸, Orn¹², Abu¹⁵, Orn²¹, Nle²⁷]hGHRH(1-29)NH-CH₃ (SEQ
ID NO: 32);
P-27406 [N-Me-Tyr¹, Gln⁸, Orn¹², Abu¹⁵, Orn²¹, Nle²⁷, Asp²⁸]hGHRH(1-29) NH-
CH₃ (SEQ ID NO: 33);
P-27407 [Dat¹, Gln⁸, Orn¹², Abu¹⁵, Orn²¹, Nle²⁷, Asp²⁸]hGHRH(1-29)NH-CH3 (SEQ
ID NO: 34);
P-27408 [Dat¹, D-Ala², Gln⁸, Orn¹², Abu¹⁵, Orn²¹, Nle²⁷, Asp²⁸]hGHRH(1-30)NH-
CH₃;
P-27409 [N-Me-Tyr¹, D-Ala², Orn¹², Abu¹⁵, Orn²¹, Nle²⁷, Asp²⁸]hGHRH(1-29)NH-
CH₃;
P-27410 [N-Me-Tyr¹, D-Ala², Thr⁸, Orn¹², Abu¹⁵, Orn²¹, Nle²⁷, Asp²⁸]hGH RH(1-
29)NH-CH₃;
P-27411 [N-Me-Tyr¹, D-Ala², Thr⁸, Orn¹², Abu¹⁵, Orn²¹, Nle²⁷]hGHRH(1-29) NH-
CH₃;
P-27412 [N-Me-Tyr¹, D-Ala², Ala⁸, Orn¹², Abu¹⁵, Orn²¹, Nle²⁷, Asp²⁸]hGHRH (1-
29)NH-CH₃;
P-27413 [Dat¹, Gln⁸, His¹¹, Orn¹², Abu¹⁵, His²⁰, Orn²¹, Nle²⁷, Asp²⁸]hGHRH (1-
29)NH-CH₃(SEQ ID NO: 35);
P-27414 [N-Me-Tyr¹, D-Ala², Fpa5⁶, Orn¹², Abu¹⁵, Orn²¹, Nle²⁷, Gab³⁰]hGHRH(1-
30)NH-CH₃;
P-27415 [N-Me-Tyr¹, Orn¹², Abu¹⁵, Orn²¹, Nle²⁷, Gab³⁰]hGHRH(1-30) NH-CH₃ (SEQ
ID NO: 36);
P-27416 [N-Me-Tyr¹, D-Ala², Gln⁸, His¹¹, Orn¹², Abu¹⁵, His²⁰, Orn²¹, Nle²⁷, Asp²⁸]
hGHRH(1-29)NH-CH₃;
P-27417 [Ac-Tyr¹, D-Ala², Gln⁸, Orn¹², Abu¹⁵, Orn²¹, Nle²⁷, Asp²⁸]hGHRH(1-29)
NH-CH₃;
P-27418 [Ac-Tyr¹, D-Ala², Fpa5⁶, Orn¹², Abu¹⁵, Orn²¹, Nle²⁷, Asp²⁸]hGHRH(1-
29)NH-CH₃;
P-27419 [Ac-Tyr¹, Thr8, Orn¹², Abu¹⁵, Orn²¹, Nle²⁷]hGHRH(1-29)NH-CH₃ (SEQ ID
NO: 37);
P-27422 [N-Me-D-Tyr¹, Gln⁸, Orn¹², Abu¹⁵, Orn²¹, Nle²⁷, Asp²⁸]hGHRH(1-29) NH-
CH₃;
P-27423 [N-Me-D-Tyr¹, D-Ala², Orn¹², Abu¹⁵, Orn²¹, Nle²⁷, Asp²⁸]hGHRH(1-29)NH-CH₃;
P-27424 [Dat¹, Thr⁸, Orn¹², Abu¹⁵, Orn²¹, Nle²⁷]hGHRH(1-29)NH-CH₃ (SEQ ID NO: 38);
P-27425 [N-Me-D-Tyr¹, D-Ala², Gln⁸, Orn¹², Abu¹⁵, Orn²¹, Nle²⁷, Asp²⁸]hGHRH(1-29)NH-
CH₃;
P-27440 [Dat¹, Gln⁸, Orn¹², Abu¹⁵, Orn²¹, Nle²⁷, Asp²⁸, D-Arg²⁹]hGHRH(1-29)NH-CH₃;
P-27441 [N-Me-Tyr¹, D-Ala², Gln⁸, Orn¹², Abu¹⁵, Orn²¹, Nle²⁷, Asp²⁸, D-Arg²⁹]hGHRH(1-
29)NH-CH₃;
P-27442 [N-Me-Tyr¹, D-Ala², Fpa5⁶, Orn¹², Abu¹⁵, Orn²¹, Nle²⁷, Asp²⁸, D-Arg²⁹]hGHRH(1-
29)NH-CH₃;
P-27443 [N-Me-Tyr¹, Orn¹², Abu¹⁵, Orn²¹, Nle²⁷, D-Arg²⁹]hGHRH(1-29)NH-CH₃;
P-27444 [N-Me-Tyr¹, D-Ala², Orn¹², Abu¹⁵, Orn²¹, Nle²⁷, Asp²⁸, D-Arg²⁹]hGHRH(1-
29)NH-CH₃;
P-27445 [N-Me-Tyr¹, D-Ala², Thr⁸, Orn¹², Abu¹⁵, Orn²¹, Nle²⁷, Asp²⁸, D-Arg²⁹]hGHRH(1-
29)NH-CH₃;
P-27446 [N-Me-Tyr¹, D-Ala², Ala⁸, Orn¹², Abu¹⁵, Orn²¹, Nle²⁷, Asp²⁸, D-Arg²⁹]hGHRH(1-
29)NH-CH₃;
P-27447 [N-Me-Tyr¹, D-Ala², Gln⁸, Orn¹², Abu¹⁵, Orn²¹, Nle²⁷, Asp²⁸, Apa³⁰]hGHRH(1-
30)NH-CH₃;
P-27448 [N-Me-Tyr¹, D-Ala², Gln⁸, Orn¹², Abu¹⁵, Orn²¹, Nle²⁷, Asp²⁸, Aha³⁰]hGHRH(1-
30)NH-CH₃;
P-27449 [N-Me-Tyr¹, D-Ala², Gln⁸, Orn¹², Abu¹⁵, Orn²¹, Nle²⁷, Asp²⁸, Amc³⁰]hGHRH(1-
30)NH-CH₃;
P-27450 [N-Me-Tyr¹, D-Ala², Gln⁸, Orn¹², Abu¹⁵, Orn²¹, Nle²⁷, Asp²⁸, Har³⁰]hGHRH(1-
30)NH-CH₃;
P-27451 [N-Me-Tyr¹, D-Ala², Gln⁸, His¹¹, Orn¹², Abu¹⁵, His²⁰, Orn²¹, Nle²⁷, Asp²⁸, Apa³⁰]
hGHRH(1-30)NH-CH₃;
P-28420 [N-Me-Tyr¹, D-Ala², Gln⁸, Orn¹², Abu¹⁵, Orn²¹, Nle²⁷, Asp²⁸]hGHRH(1-29)NH-
CH₂-CH₃;
P-28421 [N-Me-Tyr¹, D-Ala², Orn¹², Abu¹⁵, Orn²¹, Nle²⁷, Asp²⁸]hGHRH(1-29) NH-
CH₂-CH₃;
P-28430 [N-Me-Tyr¹, D-Ala², Gln⁸, Orn¹², Abu¹⁵, Orn²¹, Nle²⁷]hGHRH(1-29) NH-
CH₂-CH₃;
P-28431 [N-Me-Tyr¹, D-Ala², Thr⁸, Orn¹², Abu¹⁵, Orn²¹, Nle²⁷, Asp²⁸]hGHRH(1-
29)NH-CH₂-CH₃;
P-28460 [N-Me-Tyr¹, D-Ala², Fpa5⁶, Gln⁸, Orn¹², Abu¹⁵, Orn²¹, Nle²⁷, Asp²⁸]
hGHRH(1-29)NH-CH₂-CH₃;
P-28461 [N-Me-Tyr¹, D-Ala², Gln⁸, Orn¹², Abu¹⁵, Orn²¹, Nle²⁷]hGHRH(1-29) NH-
CH₂-CH₃;
P-28462 [N-Me-Tyr¹, D-Ala², Fpa5⁶, Orn¹², Abu¹⁵, Orn²¹, Nle²⁷]hGHRH(1-29) NH-
CH₂-CH₃;
P-28463 [N-Me-Tyr¹, D-Ala², Orn¹², Abu¹⁵, Orn²¹, Nle²⁷]hGHRH(1-29)NH-CH₂-
CH₃;
P-28464 [N-Me-Tyr¹, D-Ala², Fpa5⁶, Gln⁸, Orn¹², Abu¹⁵, Orn²¹, Nle²⁷]hGHRH (1-
29)NH-CH₂-CH₃;
P-28465 [N-Me-Tyr¹, D-Ala², Gln⁸, Orn¹², Abu¹⁵, Orn²¹, Nle²⁷, Asp²⁸] hGHRH(1-
29)NH-CH₂-CH₃;
P-28466 [N-Me-Tyr¹, D-Ala², Fpa5⁶, Orn¹², Abu¹⁵, Orn²¹, Nle²⁷, Asp²⁸]hGHRH (1-
29)NH-CH₂-CH₃;
P-28467 [N-Me-Tyr¹, D-Ala², Orn¹², Abu¹⁵, Orn²¹, Nle²⁷, Asp²⁸]hGHRH(1-29) NH-
CH₂-CH₃;
P-28468 [N-Me-Tyr¹, D-Ala², Fpa5⁶, Gln⁸, Orn¹², Abu¹⁵, Orn²¹, Nle²⁷, Asp²⁸]
hGHRH(1-29)NH-CH₂-CH₃;
P-28469 [Dat¹, D-Ala², Gln⁸, Orn¹², Abu¹⁵, Orn²¹, Nle²⁷]hGHRH(1-29)NH-CH₂-CH₃;
P-28470 [Dat¹, D-Ala², Orn¹², Abu¹⁵, Orn²¹, Nle²⁷]hGHRH(1-29)NH-CH₂-CH₃;
P-28471 [Dat¹, D-Ala², Gln⁸, Orn¹², Abu¹⁵, Orn²¹, Nle²⁷, Asp²⁸]hGHRH(1-29)NH-
CH₂-CH₃;
P-28472 [Dat¹, D-Ala², Fpa5⁶, Gln⁸, Orn¹², Abu¹⁵, Orn²¹, Nle²⁷, Asp²⁸] hGHRH(1-
29)NH-CH₂-CH₃;
P-28473 [Dat¹, D-Ala², Fpa5⁶, Orn¹², Abu¹⁵, Orn²¹, Nle²⁷, Asp²⁸]hGHRH(1-29)NH-
CH₂-CH₃;
P-28474 [Dat¹, D-Ala², Orn¹², Abu¹⁵, Orn²¹, Nle²⁷, Asp²⁸] hGHRH(1-29)NH-CH₂-
CH₃;
P-28475 [N-Me-Tyr¹, D-Ala², Gln⁸, Orn¹², Abu¹⁵, Orn²¹, Nle²⁷, Asp²⁸, Apa³⁰]hGHRH(1-
30)NH-CH₂-CH₃;
P-28476 [N-Me-Tyr¹, D-Ala², Gln⁸, Orn¹², Abu¹⁵, Orn²¹, Nle²⁷, Asp²⁸, Aha³⁰]hGHRH(1-
30)NH-CH₂-CH₃;
P-28477 [N-Me-Tyr¹, D-Ala², Gln⁸, Orn¹², Abu¹⁵, Orn²¹, Nle²⁷, Asp²⁸, Amc³⁰]hGHRH(1-
30)NH-CH₂-CH₃;
P-28478 [N-Me-Tyr¹, D-Ala², Gln⁸, Orn¹², Abu¹⁵, Orn²¹, Nle²⁷, Asp²⁸, Har³⁰]hGHRH(1-
30)NH-CH₂-CH₃;
P-28479 [N-Me-Tyr¹, D-Ala², Gln⁸, His¹¹, Orn¹², Abu¹⁵, His²⁰, Orn²¹, Nle²⁷, Asp²⁸, Apa³⁰]
hGHRH(1-30)NH-CH₂-CH₃;
P-29701 [N-Me-Tyr¹, D-Ala², Gln⁸, Orn¹², Abu¹⁵, Orn²¹, Nle²⁷, Asp²⁸, Gln-Gab³⁰] hGHRH](1-
30)NH₂;
P-29702 [Dat¹, D-Ala², Orn¹², Abu¹⁵, Orn²¹, Nle²⁷, Gln-Gab³⁰]hGHRH(1-30)NH₂;
P-29703 [N-Me-Tyr¹,Gln⁸, Orn¹², Abu¹⁵, Orn²¹, Nle²⁷, Gln-Gab³⁰]hGHRH(1-30)NH₂ (SEQ
ID NO: 39);
P-29704 [Dat¹, D-Ala², Gln⁸, Orn¹², Abu¹⁵, Orn^{21,22}, Nle²⁷, Gln-Gab³⁰]hGHRH(1-30)NH₂;
P-29706 [Tfa-Tyr¹, D-Abu², Gln⁸, Orn¹², Abu¹⁵, Orn²¹, Nle²⁷, Gln-Gab³⁰]hGHRH(1-
30)NH₂;
P-29708 [N-Me-Tyr¹, D-Ala², Fpa5⁶, Orn¹², Abu¹⁵, Orn²¹, Nle²⁷, Gln-Gab³⁰]hGHRH(1-
30)NH₂;
P-29710 [N-Me-Tyr¹, D-Ala², Fpa5⁶, Ala⁸, Orn¹², Abu¹⁵, Orn²¹, Nle27, Gln-Gab³⁰]hGHRH(1-
30)NH₂;
P-29720 [Dat¹, D-Ala², Orn¹², Abu¹⁵, Orn²¹, Nle²⁷, Asp²⁸, Gln-Gab³⁰]hGHRH(1-30)NH₂;
P-29721 [Dat¹, D-Ala², Gln⁸, Orn¹², Abu¹⁵, Orn²¹²², Nle²⁷, Asp²⁸, Gln-Gab³⁰]hGHRH(1-30)
NH₂;
P-29722 [Tfa-Tyr¹, D-Abu², Gln⁸, Orn¹², Abu¹⁵, Orn²¹, Nle²⁷, Asp²⁸, Gln³⁰, Gab³¹]hGHRH (1-
30)NH₂;
P-29723 [N-Me-Tyr¹, D-Ala², Fpa5⁶, Orn¹², Abu¹⁵, Orn²¹, Nle²⁷, Asp²⁸, Gln-Gab³⁰] hGHRH (1-
30)NH₂;
P-29724 [N-Me-Tyr¹, D-Ala², Fpa5⁶, Ala⁸, Orn¹², Abu¹⁵, Orn²¹, Nle²⁷, Asp²⁸, Gln Gab³⁰]
hGHRH(1-30)NH₂;
JI-34 [Dat¹, Orn¹², Abu¹⁵, Orn²¹, Nle²⁷, Asp²⁸, Agm²⁹]hGHRH(1-29)NH₂ (SEQ ID NO: 40);
JI-36 [Dat¹, Thr⁸, Orn¹², Abu¹⁵, Orn²¹, Nle²⁷, Asp²⁸, Agm²⁹]hGHRH(1-29)NH₂ (SEQ ID NO:
41); and
JI-38 [Dat¹, Gln⁸, Orn¹², Abu¹⁵, Orn²¹, Nle²⁷, Asp²⁸, Agm²⁹]hGHRH(1-29)NH₂ (SEQ ID NO:
42).

In some embodiments, the GHRH agonist peptide may be CJC-1295, which is represented by the formula III:

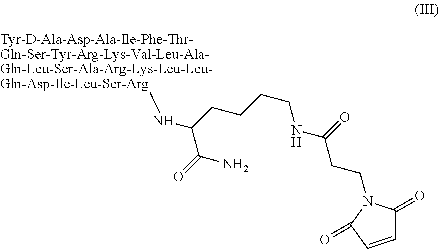

In some embodiments, the GHRH agonist peptide may be AKL-0707, represented by the abbreviated formula: [D-Ala², D-Tyr¹⁰, D-Ala¹⁵, Lys²²]hGHRH(1-29)NH₂. In some embodiments, the GHRH agonist peptide may be GHRH(1-30) represented by SEQ ID NO: 1. In some embodiments, the GHRH agonist peptide may be 1-44 amino acids of GHRH (SEQ ID NO: 43) as shown below:

(SEQ ID NO: 43)
Tyr¹-Ala²-Asp³-Ala⁴-Ile⁵-Phe⁶-Thr⁷-Asn⁸-Ser⁹-Tyr¹⁰-
Arg¹¹-Lys¹²-Val¹³-Leu¹⁴-Gly¹⁵-Gln¹⁶-Leu¹⁷-Ser¹⁸-Ala¹⁹-
Arg²⁰-Lys²¹-Leu²²-Leu²³-Gln²⁴-Asp²⁵-Ile²⁶-Met²⁷-Ser²⁸-
Arg²⁹-Gln³⁰-Gln³¹-Gly³²-Glu³³-Ser³⁴-Asn³⁵-Gln³⁶-Glu³⁷-
Arg³⁸-Gly³⁹-Ala⁴⁰-Arg⁴¹-Ala⁴²-Arg⁴³-Leu⁴⁴.

In some embodiments, the GHRH peptide may be tesamorelin. Tesamorelin is the acetate salt of a sequence having a hexenoyl moiety attached to the N-terminal part of the GHRH amino acid sequence. The peptide precursor of tesamorelin acetate is produced synthetically and is comprised of the 44 amino acid sequence of human GHRH. Tesamorelin acetate is made by attaching a hexenoyl moiety, a C6 chain with a double bond at position 3, to the tyrosine residue at the N-terminal part of the molecule. The amino acid sequence of tesamorelin, starting at the N-terminus is: Tyr¹-Ala²-Asp³-Ala⁴-Ile⁵-Phe⁶-Thr⁷-Asn⁸-Ser⁹-Tyr¹⁰-Arg¹¹-Lys¹²-Val¹³-Leu¹⁴-Gly¹⁵-Gln¹⁶-Leu¹⁷-Ser¹⁸-Ala¹⁹-Arg²⁰-Lys²¹-Leu²²-Leu²³-Gln²⁴-Asp²⁵-Ile²⁶-Met²⁷-Ser²⁸-Arg²⁹-Gln³⁰-Gln³¹-Gly³²-Glu³³-Ser³⁴-Asn³⁵-Glu³⁶-Glu³⁷-Arg³⁸-Gly³⁹-Ala⁴⁰-Arg⁴¹-Ala⁴²-Arg⁴³-Leu⁴⁴.

In some embodiments, the GHRH agonist peptides may be 1-40 amino acids in length, represented by formula IV:

(IV)
Z¹-Z²-Asp-Ala-Ile-Phe-The-Asn-Ser-Tyr-Arg-Lys-Val-
Leu-Z³-Gln-Leu-Ser-Ala-Arg-Lys-Leu-Leu-Gln-Asp-
Ile-Z⁴-Z⁵-Arg-Gln-Gln-Gly-Glu-Z⁶-Asn-Gln-Glu-Z⁷-
Gly-Ala-OH,

wherein
Z¹ is a D- or L-isomer of the aminoacid tyrosine or histidine;
Z² is a D- or L-isomer of the aminoacid alanine, valine, or isoleucine;
Z³ is a D- or L-isomer of the aminoacid alanine or glycine;
Z⁴ is a D- or L-isomer of the aminoacid methionine or leucine;
Z⁵ is a D- or L-isomer of the aminoacid serine or asparagine;
Z⁶ is a D- or L-isomer of the aminoacid arginine or serine; and
Z⁷ is a D- or L-isomer of the aminoacid glutamine or arginine.

In some embodiments, the GHRH agonist peptide may be a stapled peptide. The peptide stapling strategy for stabilizing of peptide α-helices may utilize a ring-closing metathesis (RCM) reaction. The “staple” may efficiently created in a two-step process between strategically positioned olefin functionalized non-natural amino acid side chains. The first step, catalyzed by Grubbs catalyst, may result in olefin containing bridge that is subsequently catalytically reduced to saturated hydrocarbon (alkane), effectively locking the peptide into a stable α-helix conformation. Such helix stabilization may increase the helicity, potency resistance to proteolytic degradation and cell permeability of GHRH peptides.

In some embodiments, in a non-limiting example, a method of stabilizing a α-helix in a GHRH agonist peptide may include providing a GHRH peptide containing at least two S bearing residues; and reacting the peptide with a di-halogeno-aryl-compounds to form a bis(thioether)-aryl-bridge between said two residues. The S bearing residues may be selected from (L)Cys, (D)Cys, (L)homoCys, (D)homoCys, (L)Pen(L-penicillamine), and (D)Pen.

In some embodiments, a stapled GHRH agonist peptide is represented by the formula V:

[R¹-A¹-A²-A³-A⁴-A⁵-A⁶-A⁷-A⁸-A⁹-A¹⁰-A¹¹-A¹²-A¹³-A¹⁴-A¹⁵-A¹⁶-A¹⁷-A¹⁸-A¹⁹-A²⁰-A²¹-A²²-A²³-A²⁴-A²⁵-A²⁶-A²⁷-A²⁸-A²⁹-A³⁰]-R²;
wherein
R¹ is Ac, Tfa, or is absent;
A¹ is Tyr, His, Dat, N-Me-Tyr, or an amino acid mimetic J;
A² is Ala, D-Ala, Abu, D-Abu, Val, or an amino acid mimetic J;
A³ is Asp, D-Asp, Glu, D-Glu, or an amino acid mimetic J;
A⁴ is Ala or an amino acid mimetic J;
A⁵ is Ile or an amino acid mimetic J;
A⁶ is Phe, Fpa5 or an amino acid mimetic J;
A⁷ is Thr, Aib, Leu, Trp, or an amino acid mimetic J;
A⁸ is Asn, Ala, Gln, Thr, N-Me-Ala, or an amino acid mimetic J;
A⁹ is Ser, Ala, Aib, Leu, or an amino acid mimetic J;
A¹⁰ is Tyr, D-Tyr, or an amino acid mimetic J;
A¹¹ is Arg, Ala, Gln, His, Har, or an amino acid mimetic J;
A¹² is Orn, Lys(Me)₂, Lys, Ala, Gln, or an amino acid mimetic J;
A¹³ is Val, Ile, or an amino acid mimetic J;
A¹⁴ is Leu, D-Leu, or an amino acid mimetic J;
A¹⁵ is Abu, Ala, Gly, or an amino acid mimetic J;
A¹⁶ is Gln, Ala, Aib, Glu, or an amino acid mimetic J;
A¹⁷ is Leu, D-Leu, or an amino acid mimetic J;
A¹⁸ is Ser, Tyr, or an amino acid mimetic J
A¹⁹ is Ala or an amino acid mimetic J;
A²⁰ is Arg, His, Har, Gln, or an amino acid mimetic J;
A²¹ is Orn, or Lys(Me)₂, Lys, Gln, or an amino acid mimetic J;
A²² is Leu, Orn, Ala, or an amino acid mimetic J;
A²³ is Leu, D-Leu, Ala, or an amino acid mimetic J;
A²⁴ is Gln, His, Glu, Ala, or an amino acid mimetic J
A²⁵ is Asp, Glu, D-Glu, D-Asp, or an amino acid mimetic J;
A²⁶ is Ile, Leu, or an amino acid mimetic J;
A²⁷ is Nle, Leu, Met, Ile, or an amino acid mimetic J;
A²⁸ is Ser, Asp, or an amino acid mimetic J;
A²⁹ is Arg, Har, Agm, D-Arg, D-Har, or an amino acid mimetic J;
A³⁰ is Arg, Agm, Ada, Amc, Aha, Apa, Har, D-Arg, D-Har, Gab, Gln, D-Gln, Gln-Gab, D-Gln-Gab, or is absent;
each peptide has at least two amino acid mimetic J, and J is represented by the formula VI

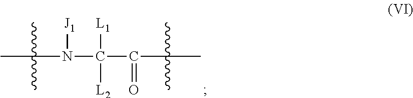

each J₁ is, independently, H, alkyl, alkenyl, alkynyl, arylalkyl, cycloalkyl, heteroalkyl, cycloalkylalkyl, heterocycloalkyl, cycloaryl, aryl, or heterocycloaryl;
each L₁ and L₂ is independently, alkyl, alkenyl, alkynyl, heteroalkyl, cycloalkyl, arly, heterocycloalkyl, cycloaryl, heterocycloaryl, or
each L₁ and L₂ of first amino acid mimetic J is connected to a second amino acid mimetic J to form a linker, and the linker is selected from alkylene, alkenylene, alkynylene, heteroalkylene, cycloalkylene, heterocycloalkylene, cycloarylene, heterocycloarylene, or [-J₃-U-J₃-]n;
each J₃ is independently, alkylene, alkenylene, alkynylene, heteroalkylenecycloalkylene, heterocycloalkylene, arylene, or heteroarylene;
each U is O, S, SO, SO₂, CO, CO₂, or CONJ₃; and
n is an integer from 1 to 10;
In some embodiments, the agonist peptide is different from SEQ ID NO: 1 in at least one amino acid residue.

The stapled GHRH agonist peptides may contain at least two amino acid mimetics of formula VI that together form an intramolecular cross-link that help to stabilize the alpha-helical secondary structures of a portion of GHRH that is thought to be important for agonist activity at the GHRH receptor. In some embodiments, the stapled GHRH agonist peptide may comprise one, two, three, four, five or more amino acid mimetic J of formula VI, wherein each amino acid mimetic connects to another amino acid mimetic formuing a linker within the GHRH agonist peptide. For example, a GHRH agonist peptide may comprise at least two linkers wherein the first linker connects a first amino acid mimetic J to a second amino acid mimetic J, and the second linker connects a third amino acid mimetic J to a fourth amino acid mimetic J. In some embodiments, a GHRH agonist peptide comprises exactly two linkers. In other embodiments, a GHRH agonist peptide comprises exactly one linker. In some embodiments, the amino acid mimetic J can connect to two amino acid mimetics J that are present on either side of it.

The linkers can connect any two amino acid mimetics J without impairing the activity of the GHRH agonist peptide. In some embodiments, a linker connects one of the following pairs of amino acid mimetics that are present at the following positions (numbered with reference to any sequences aligned to GHRH 1-29): 4 and 8; 5 and 12; 8 and 12; 8 and 15; 9 and 16; 12 and 16; 12 and 19; 15 and 22; 18 and 25; 21 and 25; 21 and 28; 22 and 29; 25 and 29.

In some embodiments, the GHRH agonist peptide may have more than one linker moiety in the peptide. For example, a pair of linkers may connect amino acid mimetics at positions 4 and 8, and 8 and 12. In some embodiments, a GHRH agonist peptide may comprise 3 linkers. In some embodiments, a GHRH agonist peptide may comprise 4 linkers. In some embodiments, a GHRH agonist peptide may comprise 5 linkers.

The various GHRH agonist peptides disclosed herein may all act in a similar mechanism to bring about the biological effect. For example, various GHRH agonist peptides disclosed herein may all bind to the GHRH receptors that are present on pituatory and non-pituatory tissues and active the similar signaling pathways and bring about the same biochemical changes. Thus, a GHRH agonist peptide such as JI-34 may work in a similar manner to a GHRH agonist peptide P-20103 or any other GHRH peptide belonging to formulae I-V. For example, the peptides, such as P-20356, P-27403, P-27409, P-25502, P-20350, P-28421, P-28420, P-27406, P-20361, P-20367, JI-36, and JI-38 may have growth inhibitory effect on a cancer cell similar to a JI-34 peptide.

Disclosed herein are methods to treat a subject with cancer. In some embodiments, a method of treating a subject with cancer may comprise administering a therapeutically effective amount of at least one GHRH agonist peptide described herein in combination with at least one anticancer agent. In some embodiments, the cancer may be colorectal cancer, breast cancer, ovarian cancer, pancreatic cancer, head and neck cancer, bladder cancer, liver cancer, renal cancer, melanoma, gastrointestinal cancer, prostate cancer, small cell and non-small cell lung cancer, sarcomas, glioblastoma, T- and B-cell lymphoma, endometrial cancer, and cervical cancer.

In some embodiments, a GHRH agonist peptide of formula I is used in combination with an anticancer agent described herein to treat a subject having cancer. In some embodiments, a GHRH agonist peptide of formula II is used in combination with an anticancer agent described herein to treat a subject having cancer. In some embodiments, a GHRH agonist peptide of formula III (CJC-1295) is used in combination with an anticancer agent described herein to treat a subject having cancer. In some embodiments, a GHRH agonist peptide of formula IV is used in combination with an anticancer agent described herein to treat a subject having cancer. In some embodiments, a GHRH agonist peptide of formula V is used in combination with an anticancer agent described herein to treat a subject having cancer. In some embodiments, tesamorelin is used in combination with an anticancer agent described herein to treat a subject having cancer. In some embodiments, a combination of GHRH agonist peptides may be used along with an anticancer agent described herein to treat a subject having cancer. For example, a combination of GHRH agonist peptides of formula I and formula III may be used along with an anticancer agent. In other embodiments, a combination of GHRH agonist peptide of formula I and tesamorelin may be used along with an anticancer agent. In yet another embodiment, a combination of GHRH agonist peptides of formula I and formula IV may be used along with an anticancer agent. In a further embodiment, a combination of GHRH agonist peptides of formula II and formula III may be used along with an anticancer agent.

In some embodiments, the GHRH agonist peptides in combination with an anticancer agent, may be used treat glioblastomas. Glioblastoma multiforme (GBM) is one of the most aggressive human cancers, and the afflicted patients inevitably succumb. The dismal outcome of this malignancy demands great efforts to find improved methods of treatment. Cancers of the brain include, but are not limited to, oligodendrogliomas and glioblastomas including glioblastoma multiforme (GBM). Tissues affected by the cancerous cells can be in the brain itself (e.g., the cranium or the central spinal canal) or in lymphatic tissue, in blood vessels, in the cranial nerves, in the brain envelopes (meninges), skull, pituitary gland, or pineal gland. Specific forms of brain cancer that can be treated include astrocytomas, chondromas, chondrosarcomas, chordomas, CNS (central nervous system) lymphomas, craniopharyngiomas, ependymomas, gangliogliomas, ganglioneuromas (also called gangliocytomas), gliomas, including astrocytomas, oligodendrogliomas, ependymomas, hemangioblastomas (also called vascular tumors), and primitive neuroectodermal tumors (PNET), such as medulloblastomas, meningiomas, and vestibular schwannomas (formerly known as acoustic neuroma).

In preferred non-limiting embodiments, the cancer is amenable to treatment by direct administration of the GHRH agonist peptide. For example, a target tumor mass may be close to the surface of the skin. In another example, a diseased tissue may be encapsulated by a cyst, or is found in a substantially enclosed cavity including, without limitation, a lumen. In other embodiments, the cancer is amenable to treatment by intravenous administration of the GHRH agonist peptide.

The invention also provides methods for reducing the risk of post-surgical complications comprising administering an effective amount of the GHRH agonist peptide before, during, or after surgery, and in specific non-limiting embodiments, surgery to treat cancer.

The invention also provides methods for preventing occurrence, preventing or delaying recurrence, or reducing the rate of recurrence of a cancer comprising directly administering to a patient in need thereof an effective amount of at least one GHRH agonist peptide described herein in combination with at least one anticancer agent.

The invention also provides methods for sensitizing a tumor or cancer to one or more other anticancer agents comprising administering at least one GHRH agonist peptide of the invention. The anticancer agents may be administered prior to, overlapping with, concurrently, and/or after administration of the GHRH agonist peptide. In some embodiments, at least one GHRH agonist peptide is administered to the subject before the cancer treatment, concurrently with the cancer treatment, post-treatment, or during remission of the cancer. When administered concurrently, the GHRH agonist peptide and other anticancer agent may be administered in a single formulation or in separate formulations, and if separately, then optionally, by different modes of administration. Accordingly, the combination of one or more GHRH agonist peptides and one or more other anticancer agents may synergistically act to combat the tumor or cancer.

In some embodiments, the anticancer agents may be tamoxifen, toremifen, raloxifene, droloxifene, iodoxyfene, megestrol acetate, anasfrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, vincristine, taxol, taxotere, etoposide, teniposide, amsacrine, Irinotecan, topotecan, an epothilone, gefitinib, erlotinib, sorafenib, angiogenesis inhibitors, EGF inhibitors, VEGF inhibitors, CDK inhibitors, cytokines, Her1 and Her2 inhibitors, and monoclonal antibodies.

In another embodiment, GHRH agonist peptide is administered in combination with a regimen of radiation therapy. The therapy may also comprise surgery and/or chemotherapy. For example, the GHRH agonist peptide may be administered in combination with radiation therapy and cisplatin (Platinol), fluo-rouracil (5-FU, Adrucil), carboplatin (Paraplatin), and/or paclitaxel (Taxol). Treatment with the GHRH agonist peptide may allow use of lower doses of radiation and/or less frequent radiation treatments, which may for example, reduce the incidence of severe sore throat that impedes swallowing function potentially resulting in undesired weight loss or dehydration.

Where the GHRH agonist peptide of the invention is administered in addition to one or more other anticancer agents, these other anticancer agents may include, without limitation, 2,2′,2″trichlorotriethylamine, 6-azauridine, 6-diazo-5-oxo-L-norleucine, mercaptopurine, aceglarone, aclacinomycinsa actinomycin, altretamine, aminoglutethimide, amsacrine, anastrozole, ancitabine, angiogenin antisense oligonucleotide, anthramycin, azacitidine, azaserine, aziridine, batimastar, bcl-2 antisense oligonucleotide, benzodepa, bicalutamide, bisantrene, bleomycin, buserelin, busulfan, cactinomycin, calusterone, carboplatin, carboquone, carmofur, carmustine, carubicin, carzinophilin, chlorambucil, chloraphazine, chlormadinone acetate, chlorozotocin, chromomycins, cisplatin, cladribine, cyclophosphamide, cytarabine, dacarbazine, dactinomycin, daunorubicin, defosfamide, demecolcine, denopterin, diaziquone, docetaxel, doxifluridine, doxorubicin, droloxifene, dromo-stanolone, edatrexate, eflornithine, elliptinium acetate, emitefur, enocitabune, epirubicin, epitiostanol, estramustine, etoglucid, etoposide, fadrozole, fenretinide, floxuridine, fludarabine, fluorouracil, flutamide, folinic acid, formestane, fosfestrol, fotemustine, gallium nitrate, gemcitabine, goserelin, hexestrol, hydroxyurea, idarubicin, ifosfamide, improsulfan, interferonalpha, interferonbeta, interferon-gamma, interleukin-2, L-asparaginase, lentinan, letrozole, leuprolide, lomustine, lonidamine, mannomustine, mechlorethamine, mechlorethamine oxide hydrochloride, medroxyprogesterone, megestrol acetate, melengestrol, melphalan, menogaril, mepitiostane, methotrexate, meturedepa, miboplatin, miltefosine, mitobronitol, mitoguazone, mitolactol, mitomycins, mitotane, mitoxantrone, mopidamol, mycophenolic acid, nilutamide, nimustine, nitracine, nogalamycin, novembichin, olivomycins, oxaliplatin, paclitaxel, pentostain, peplomycin, perfosfamide, phenamet, phenesterine, pipobroman, piposulfan, pirarubicin, piritrexim, plicamycin, podophyllinic acid 2-ethyl-hydrazide, polyestradiol phosphate, porfimer sodium, porfiromycin, prednimustine, procabazine, propagermanium, PSK, pteropterin, puromycin, ranimustine, razoxane, roquinimex, sizofican, sobuzoxane, spirogerma-nium, streptonigrin, streptozocin, tamoxifen, tegafur, temozolomide, teniposide, tenuzonic acid, testolacone, thiamiprine, thioguanine, Tomudex, topotecan, toremifene, triaziquone, triethylenemelamine, triethylenephosphoramide, triethylenethiophosphoramide, trilostane, trimetrexate, triptorelin, trofosfamide, trontecan, tubercidin, ubenimex, uracil mustard, uredepa, urethan, vincristine, zinostatin, zorubicin, cytosine arabinoside, gemtuzumab, thioepa, cyclothosphamide, antimetabolites (e.g., methotrexate, 6-mercaptopurine, 6-thioguanine, cytarabine, 5-fluorouracil, fludarabine, gemcitabine, dacarbazine, temozoamide), hexamethylmelamine, LYSODREN, nucleoside analogues, plant alkaloids (e.g., Taxol, paclitaxel, camptothecin, topotecan, irinotecan (CAMPTOSAR,CPT-11), vinca alkyloids such as vinblastine, podophyllotoxin, epipodophyllotoxin, VP-16 (etoposide), cytochalasin B, gramicidin D, ethidium bromide, emetine, anthracyclines, liposomal doxorubicin, dihydroxyanthracindione, mithramycin, actinomycin D, aldesleukin, allutamine, biaomycin, capecitabine, carboplain, chlorabusin, cyclarabine, daclinomycin, floxuridhe, lauprolide acetate, levamisole, lomusline, mercaptopurino, mesna, mitolanc, pegaspergase, pentoslatin, picamycin, riuxlmab, campath-1, straplozocin, tretinoin, VEGF antisense oligonucleotide, vindesine, and vinorelbine. Compositions comprising one or more anticancer agents (e.g., FLAG, CHOP) are also contemplated by the present invention. FLAG comprises fludarabine, cytosine arabinoside (Ara-C) and G-CSF. CHOP comprises cyclophosphamide, vincristine, doxorubicin, and prednisone. Likewise, the GHRH agonist peptide of the invention may be used in conjunction with radiation therapy or other known anticancer modalities.

Pharmaceutical compositions for combination therapy may also include, without limitation, antibiotics (e.g., dactinomycin, bleomycin, mithramycin, anthramycin), asparaginase, BCG protein, diphtheria toxin, procaine, tetracaine, lidocaine, propranolol, anti-mitotic agents, abrin, ricin A, Pseudomonas exotoxin, nerve growth factor, platelet derived growth factor, tissue plasminogen activator, antihistaminic agents, anti-nausea agents, etc.

Indeed, direct administration of an effective amount of an GHRH agonist peptide to a patient in need of such treatment may result in reduced doses of another anticancer agent having clinically significant efficacy. Such efficacy of the reduced dose of the other anticancer agent may not be observed absent administration with GHRH agonist peptide. Accordingly, the present invention provides methods for treating a tumor or cancer comprising administering a reduced dose of one or more other anticancer agents.

Moreover, combination therapy comprising GHRH agonist peptide to a patient in need of such treatment may permit relatively short treatment times when compared to the duration or number of cycles of standard treatment regimens. Accordingly, the present invention provides methods for treating a tumor or cancer comprising administering one or more other anticancer agents for relatively short duration and/or in fewer treatment cycles.

Thus, in accordance with the present invention, combination therapies comprising GHRH agonist peptide and another anticancer agent may reduce toxicity (i.e., side effects) of the overall cancer treatment. For example, reduced toxicity, when compared to a monotherapy or another combination therapy, may be observed when delivering a reduced dose of GHRH agonist peptide and/or other anticancer agent, and/or when reducing the duration of a cycle (i.e., the period of a single administration or the period of a series of such administrations), and/or when reducing the number of cycles.

In a preferred embodiment, the invention provides methods for treating and/or ameliorating the clinical condition of patients suffering from glioblastoma. Accordingly, the invention provides methods for: (i) decreasing the tumor size, growth rate, invasiveness, malignancy grade, and/or risk of recurrence; (ii) prolonging the disease-free interval following treatment; and (iii) decreasing metastatic potential of the glioblastoma by administering to the patient an effective amount of GHRH agonist peptide in combination with an anticancer agent.

Clinical outcomes of cancer treatments using GHRH agonist peptide of the invention are readily discernible by one of skill in the relevant art, such as a physician. For example, standard medical tests to measure clinical markers of cancer may be strong indicators of the treatment's efficacy. Such tests may include, without limitation, physical examination, performance scales, disease markers, 12-lead ECG, tumor measurements, tissue biopsy, cytoscopy, cytology, longest diameter of tumor calculations, radiography, digital imaging of the tumor, vital signs, weight, recordation of adverse events, assessment of infectious episodes, assessment of concomitant medications, pain assessment, blood or serum chemistry, detecting serum markers, urinalysis, CT scan, and pharmacokinetic analysis. Furthermore, synergistic effects of a combination therapy comprising the GHRH agonist peptide and another anticancer agent may be determined by comparative studies with patients undergoing monotherapy.

The effective dose of GHRH agonist peptide to be administered during a cycle varies according to the mode of administration. Direct administration (e.g., intratumoral injection) requires much smaller total body doses of GHRH agonist peptide as compared to systemic, intravenous administration of the GHRH agonist peptide. It will be evident to the skilled artisan that local administration can result in lower body doses, and in those circumstances, and resulting low circulating plasma level of GHRH agonist peptide would be expected and desired.

In one specific non-limiting embodiment, a GHRH agonist peptide is administered at a dose of approximately 280 micrograms/tumor/day, wherein the patient is administered a single dose per day. The maximum injection volume in a single dose is approximately one-third of the estimated target tumor volume. The single dose is administered every day for approximately five consecutive days. After this cycle, a subsequent cycle may begin approximately one month later, preferably one month from the first day of the first cycle. The treatment regime may include three cycles, each cycle being spaced apart by approximately one treatment-free week.

In another specific non-limiting embodiment, a GHRH agonist peptide is administered at a dose of approximately 280 micrograms/tumor/day, wherein the patient is administered a single dose per day. The maximum injection volume in a single dose is approximately one-third of the estimated target tumor volume. The single dose is administered every other day for approximately one week. After this cycle, a subsequent cycle may begin approximately one week later. The treatment regime may include three cycles, each cycle being spaced apart by approximately one week.

In yet another specific embodiment, a GHRH agonist peptide is administered at a dose of approximately 280 micrograms/tumor/day, wherein the patient is administered a single dose per day. The maximum injection volume in a single dose is approximately one-third of the estimated target tumor volume. The single dose is administered every other day for approximately three weeks. After this cycle, a subsequent cycle may begin approximately one week later. The treatment regime may include three cycles, each cycle being spaced apart by approximately one week.

For administration to a cavity such as peritoneal cavity, the effective dose of the GHRH agonist peptide is between about 100 and 2000 micrograms in 50 ml/week, for example approximately 100, 200, 335, 500, 700, 930, 1240 micrograms in 50 ml/week, wherein the patient is administered a single dose per week and the tumor tissue is exposed to the GHRH agonist peptide for at least about 30 minutes. For example, the solution is retained into the cavity for about 30 minutes to about 3 hours. In a specific non-limiting embodiment, the tumor tissue is exposed to the GHRH agonist peptide for about 1 hours or more preferably for about 2 hours. After this cycle, a subsequent cycle may begin approximately 1, 2, 4, 6, or 12 weeks after the previous dose. The treatment regime may include 1, 2, 3, 4, 5, or 6 cycles, each cycle being spaced apart by approximately 1, 2, 4, 6, or 12 months.

The effective dose of another anticancer agent to be administered together with GHRH agonist peptide during a cycle also varies according to the mode of administration. The one or more anticancer agent may be delivered intratumorally, or by other modes of administration. Typically, chemotherapeutic agents are administered systemically. Standard dosage and treatment regimens are known in the art (see, e.g., the latest editions of the Merck Index and the Physician's Desk Reference).

For example, in one embodiment, the additional anticancer agent comprises dacarbazine at a dose ranging from approximately 200 to 4000 mg/m²/cycle. In a preferred embodiment, the dose ranges from 700 to 1000 mg/m²/cycle.

In another embodiment, the additional anticancer agent comprises fludarabine at a dose ranging from approximately 25 to 50 mg/m²/cycle.

In another embodiment, the additional anticancer agent comprises cytosine arabinoside (Ara-C) at a dose ranging from approximately 200 to 2000 mg/m²/cycle.

In another embodiment, the additional anticancer agent comprises docetaxel at a dose ranging from approximately 1.5 to 7.5 mg/kg/cycle.

In another embodiment, the additional anticancer agent comprises paclitaxel at a dose ranging from approximately 5 to 15 mg/kg/cycle.

In yet another embodiment, the additional anticancer agent comprises cisplatin at a dose ranging from approximately 5 to 20 mg/kg/cycle.

In yet another embodiment, the additional anticancer agent comprises 5-fluorouracil at a dose ranging from approximately 5 to 20 mg/kg/cycle.

In yet another embodiment, the additional anticancer agent comprises doxorubicin at a dose ranging from approximately 2 to 8 mg/kg/cycle.

In yet another embodiment, the additional anticancer agent comprises epipodophyllotoxin at a dose ranging from approximately 40 to 160 mg/kg/cycle.

In yet another embodiment, the additional anticancer agent comprises cyclophosphamide at a dose ranging from approximately 50 to 200 mg/kg/cycle.

In yet another embodiment, the additional anticancer agent comprises irinotecan at a dose ranging from approximately 50 to 75, 75 to 100, 100 to 125, or 125 to 150 mg/m²/cycle.

In yet another embodiment, the anticancer agent comprises vinblastine at a dose ranging from approximately 3.7 to 5.4, 5.5 to 7.4, 7.5 to 11, or 11 to 18.5 mg/m²/cycle.

In yet another embodiment, the additional anticancer agent comprises vincristine at a dose ranging from approximately 0.7 to 1.4, or 1.5 to 2 mg/m²/cycle.

In yet another embodiment, the additional anticancer agent comprises methotrexate at a dose ranging from approximately 3.3 to 5, 5 to 10, 10 to 100, or 100 to 1000 mg/m²/cycle.

Combination therapy with a GHRH agonist peptide may sensitize the cancer or tumor to administration of an additional anticancer agent. Accordingly, the present invention contemplates combination therapies for preventing, treating, and/or preventing recurrence of cancer comprising administering an effective amount of a GHRH agonist peptide prior to, subsequently, or concurrently with a reduced dose of an anticancer agent. For example, initial treatment with a GHRH agonist peptide may increase the sensitivity of a cancer or tumor to subsequent challenge with a dose of anticancer agent. This dose is near, or below, the low range of standard dosages when the anticancer agent is administered alone, or in the absence of a GHRH agonist peptide. When concurrently administered, the GHRH agonist peptide may be administered separately from the anticancer agent, and optionally, via a different mode of administration.

Accordingly, in one embodiment, the additional anticancer agent comprises cisplatin, e.g., PLATINOL or PLATINOL-AQ (Bristol Myers), at a dose ranging from approximately 5 to 10, 11 to 20, 21 to 40, or 41 to 75 mg/m²/cycle.

In another embodiment, the additional anticancer agent comprises carboplatin, e.g., PARAPLATIN (Bristol Myers), at a dose ranging from approximately 2 to 3, 4 to 8, 9 to 16, 17 to 35, or 36 to 75 mg/m²/cycle.

In another embodiment, the additional anticancer agent comprises cyclophosphamide, e.g., CYTOXAN (Bristol Myers Squibb), at a dose ranging from approximately 0.25 to 0.5, 0.6 to 0.9, 1 to 2, 3 to 5, 6 to 10, 11 to 20, or 21 to 40 mg/kg/cycle.

In another embodiment, the additional anticancer agent comprises cytarabine, e.g., CYTOSAR-U (Pharmacia & Upjohn), at a dose ranging from approximately 0.5 to 1, 2 to 4, 5 to 10, 11 to 25, 26 to 50, or 51 to 100 mg/m²/cycle. In another embodiment, the additional anticancer agent comprises cytarabine liposome, e.g., DEPOCYT (Chiron Corp.), at a dose ranging from approximately 5 to 50 mg/m²/cycle.

In another embodiment, the additional anticancer agent comprises dacarbazine, e.g., DTIC or DTICDOME (Bayer Corp.), at a dose ranging from approximately 15 to 250 mg/m2/cycle or ranging from approximately 0.2 to 2 mg/kg/cycle.

In another embodiment, the additional anticancer agent comprises topotecan, e.g., HYCAMTIN (SmithKline Beecham), at a dose ranging from approximately 0.1 to 0.2, 0.3 to 0.4, 0.5 to 0.8, or 0.9 to 1.5 mg/m²/Cycle.

In another embodiment, the additional anticancer agent comprises irinotecan, e.g., CAMPTOSAR (Pharmacia & Upjohn), at a dose ranging from approximately 5 to 9, 10 to 25, or 26 to 50 mg/m²/cycle.

In another embodiment, the additional anticancer agent comprises fludarabine, e.g., FLUDARA (Berlex Laboratories), at a dose ranging from approximately 2.5 to 5, 6 to 10, 11 to 15, or 16 to 25 mg/m²/cycle.

In another embodiment, the additional anticancer agent comprises cytosine arabinoside (Ara-C) at a dose ranging from approximately 200 to 2000 mg/m2/cycle, 300 to 1000 mg/m2/cycle, 400 to 800 mg/m2/cycle, or 500 to 700 mg/m²/cycle.

In another embodiment, the additional anticancer agent comprises docetaxel, e.g., TAXOTERE (Rhone Poulenc Rorer) at a dose ranging from approximately 6 to 10, 11 to 30, or 31 to 60 mg/m²/cycle.

In another embodiment, the additional anticancer agent comprises paclitaxel, e.g., TAXOL (Bristol Myers Squibb), at a dose ranging from approximately 10 to 20, 21 to 40, 41 to 70, or 71 to 135 mg/kg/cycle.

In another embodiment, the additional anticancer agent comprises 5-fluorouracil at a dose ranging from approximately 0.5 to 5 mg/kg/cycle, 1 to 4 mg/kg/cycle, or 2-3 mg/kg/cycle.

In another embodiment, the additional anticancer agent comprises doxorubicin, e.g., ADRIAMYCIN (Pharmacia & Upjohn), DOXIL (Alza), RUBEX (Bristol Myers Squibb), at a dose ranging from approximately 2 to 4, 5 to 8, 9 to 15, 16 to 30, or 31 to 60 mg/kg/cycle.

In another embodiment, the additional anticancer agent comprises etoposide, e.g., VEPESID (Pharmacia & Upjohn), at a dose ranging from approximately 3.5 to 7, 8 to 15, 16 to 25, or 26 to 50 mg/m²/cycle.

In another embodiment, the additional anticancer agent comprises vinblastine, e.g., VELBAN (Eli Lilly), at a dose ranging from approximately 0.3 to 0.5, 0.6 to 0.9, 1 to 2, or 3 to 3.6 mg/m²/cycle.

In another embodiment, the additional anticancer agent comprises vincristine, e.g., ONCOVIN (Eli Lilly), at a dose ranging from approximately 0.1, 0.2, 0.3, 0.4, 0.5, 0.6 or 0.7 mg/m²/cycle.

In another embodiment, the additional anticancer agent comprises methotrexate at a dose ranging from approximately 0.2 to 0.9, 1 to 5, 6 to 10, or 11 to 20 mg/m²/cycle.

In another embodiment, a GHRH agonist peptide is administered in combination with at least one other immunotherapeutic which includes, without limitation, rituxan, rituximab, campath-1, gemtuzumab, and trastuzutmab.

In another embodiment, a GHRH agonist peptide is administered in combination with one or more anti-angiogenic agents which include, without limitation, angiostatin, thalidomide, kringle 5, endostatin, Serpin (Serine Protease Inhibitor), anti-thrombin, 29 kDa N-terminal and a 40 kDa C-terminal proteolytic fragments of fibronectin, 16 kDa proteolytic fragment of prolactin, 7.8 kDa proteolytic fragment of platelet factor-4, a 13 amino acid peptide corresponding to a fragment of platelet factor-4, a 14-amino acid peptide corresponding to a fragment of collagen I, a 19 amino acid peptide corresponding to a fragment of thrombospondin I, a 20-amino acid peptide corresponding to a fragment of SPARC, and a variant thereof, including a pharmaceutically acceptable salt thereof.

In another embodiment, a GHRH agonist peptide is administered in combination with one or more cytokines which include, without limitation, a lymphokine, tumor necrosis factors, tumor necrosis factor-like cytokine, lymphotoxin, interferon, macrophage inflammatory protein, granulocyte monocyte colony stimulating factor, interleukin (including, without limitation, interleukin-1, interleukin-2, interleukin-6, interleukin-12, interleukin-15, interleukin-18), and a variant thereof, including a pharmaceutically acceptable salt thereof.

In yet another embodiment, a GHRH agonist peptide is administered in combination with a cancer vaccine including, without limitation, autologous cells or tissues, non-autologous cells or tissues, carcinoembryonic antigen, alpha-feto-protein, human chorionic gonadotropin, BCG live vaccine, melanocyte lineage proteins, and mutated, tumor-specific antigens.

In yet another embodiment, a GHRH agonist peptide is administered in association with hormonal therapy. Hormonal therapeutics include, without limitation, a hormonal agonist, hormonal antagonist (e.g., flutamide, tamoxifen, leuprolide acetate (LUPRON)), and steroid (e.g., dexamethasone, retinoid, betamethasone, cortisol, cortisone, prednisone, dehydrotestosterone, glucocorticoid, mineralocorticoid, estrogen, testosterone, progestin).

In yet another embodiment, a GHRH agonist peptide is administered in association with a gene therapy program to treat or prevent cancer.

Also disclosed herein are methods to treat a subject with cancer by administering GHRH peptides alone. In some embodiments, a method of treating a subject with cancer may comprise administering a therapeutically effective amount of at least one GHRH agonist peptide described herein to the subject. In some embodiments, the cancer may be colorectal cancer, breast cancer, ovarian cancer, pancreatic cancer, head and neck cancer, bladder cancer, liver cancer, renal cancer, melanoma, gastrointestinal cancer, prostate cancer, small cell and non-small cell lung cancer, sarcomas, glioblastoma, T- and B-cell lymphoma, endometrial cancer, and cervical cancer. In some embodiments, the administration of the at least one GHRH agonist peptide is by parenteral administration, such as subcutaneous, intramuscular, intraperitoneal, intracavity, intrathecal, transdermal and intravenous injection. In other embodiments, the GHRH agonist peptide may be any agonist peptide described herein, such as GHRH (1-30) represented by SEQ ID NO: 1, GHRH(1-44) represented by SEQ ID NO: 43, GHRH agonist peptide of formula I, GHRH agonist peptide of formula II, GHRH agonist peptide of formula III, GHRH agonist peptide of formula IV, GHRH agonist peptide of formula V, tesamorelin, and any combination thereof.

Disclosed herein are methods to kill cancer cells in vivo or in vitro. In some embodiments, a method of killing cancer cells may include contacting the cancer cells with a composition comprising at least one GHRH agonist peptide. In some embodiments, the cancer cells may be contacted with GHRH agonist peptide in combination with at least one anticancer agent. The GHRH agonist peptide may be any GHRH peptide disclosed herein. Non-limiting examples of cancer cell include a colorectal cancer cell, a breast cancer cell, an ovarian cancer cell, a pancreatic cancer cell, a head and neck cancer cell, a bladder cancer cell, a liver cancer cell, a renal cancer cell, a melanoma cell, a gastrointestinal cancer cell, a prostate cancer cell, a small cell lung cancer cell, non-small cell lung cancer cell, a sarcoma cell, a glioblastoma cell, T- and B-cell lymphoma cell, a endometrial cancer cell, and a cervical cancer cell.

Also disclosed herein are method to kill cancer stem cells. Many cancer cells become resistant to current therapies of chemotherapy and radiation, and a small group of cells persist even after extensive treatment. One hypothesis to explain this resistance is the presence of cancer stem cells. Not wishing to be bound by theory, a distinct subset of cells within each tumor are capable of indefinite self-renewal and can develop into adult tumor cell(s), which are relatively limited in replication capacity. It has been hypothesized that these cancer stem cells (CSC) might be more resistant to chemotherapeutic agents, radiation or other toxic conditions, and thus, persist after clinical therapies and later grow into secondary tumors, metastases or be responsible for relapse. It has been suggested that CSCs can arise either from the tissue stem cells or from a more differentiated tissue progenitor cell(s).

In some embodiments, a method of killing cancer stem cells may comprise contacting the cancer stem cells with a composition comprising at least one GHRH agonist peptide. In some embodiments, the cancer stem cells may be contacted with GHRH agonist peptide in combination with at least one anticancer agent. The GHRH agonist peptide may be any GHRH peptide disclosed herein.

The GHRH agonists of the invention may be administered in the form of pharmaceutically acceptable, nontoxic salts, such as acid addition salts. Illustrative of such acid addition salts are hydrochloride, hydrobromide, sulphate, phosphate, fumarate, gluconate, tannate, maleate, acetate, trifluoroacetate, citrate, benzoate, succinate, alginate, pamoate, malate, ascorbate, tartarate, and the like. Particularly preferred agonists are salts of low solubility, e.g., pamoate salts and the like. These exhibit long duration of activity.

Formulations containing the GHRH agonists of the present invention and a suitable carrier can be solid dosage forms which include, but are not limited to, softgels, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. In some embodiments, a single dose may comprise one or more softgels, tablets, capsules, cachets, pellets, pills, or the like. Specific examples include, for example, a dose comprising 1, 2, 3, or 4 softgels, tablets, capsules, cachets, pellets, pills or the like.

In some embodiments, one or more softgels, tablets, capsules, cachets, pellets, pills, or the like can be taken to achieve the desired dosing. In some embodiments, one or more softgels, tablets, capsules, cachets, pellets, pills, or the like can be taken simultaneously to achieve the desired dosing. In yet another embodiment one or more softgels, tablets, capsules, cachets, pellets, pills, or the like can be taken separately during the course of a specified time period such as for example, a 24 hour period. For example, one or more softgels, tablets, capsules, cachets, pellets, pills, or the like can be taken twice in a 24 hour period to achieve the desired dose. In some embodiments, one or more softgels, tablets, capsules, cachets, pellets, pills, or the like can be taken with a meal. For example one or more softgels, tablets, capsules, cachets, pellets, pills, or the like can be taken with each meal during the course of a 24 hour period to achieve the desired dose.

It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

In some embodiments, the pharmaceutical excipient may include, without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like. Pharmaceutical compositions of the peptides/compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The peptides/compounds of the present invention can be administered in the conventional manner by any route where they are active. Administration can be systemic, parenteral, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the peptides/compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Alternatively, the GHRH agonists may be administered as an intranasal spray with an appropriate carrier or by pulmonary inhalation. One suitable route of administration is a depot form formulated from a biodegradable suitable polymer, e.g., poly-D,L-lactide-coglycolide as microcapsules, microgranules or cylindrical implants containing dispersed agonistic compounds.

Specific modes of administration will depend on the indication. The selection of the specific route of administration and the dose regimen is to be adjusted or titrated by the clinician according to methods known to the clinician in order to obtain the optimal clinical response. The amount of compounds to be administered is that amount which is therapeutically effective. The dosage to be administered will depend on the characteristics of the subject being treated, e.g., the particular animal or human being treated, age, weight, health, types of concurrent treatment, if any, and frequency of treatments, and can be easily determined by one of skill in the art (e.g., by the clinician).

The amount of GHRH agonists needed depends on the type of pharmaceutical composition and on the mode of administration. In cases where human subjects receive solutions of GHRH agonists, administered by i.m. or s.c. injection, or in the form of intranasal spray or pulmonary inhalation, the typical doses are between 2-20 mg/day/patient, given once a day or divided into 2-4 administrations/day. When the GHRH agonists are administered intravenously to human patients, typical doses are in the range of 8-80 μg/kg of body weight/day, divided into 1-4 bolus injections/day or given as a continuous infusion. When depot preparations of the GHRH agonists are used, e.g. by i.m. injection of pamoate salts or other salts of low solubility, or by i.m. 10 or s.c. administration of microcapsules, microgranules, or implants containing the agonistic compounds dispersed in a biodegradable polymer, the typical doses are between 1-10 mg agonist/day/patient.

The GHRH agonists of the present invention can be formulated for parenteral administration by injection, e.g., by bolus injection or continuous infusion. The peptides/compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the GHRH agonists can be formulated readily by combining these peptides/compounds with pharmaceutically acceptable carriers well known in the art. Such carriers enable the peptides/compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active peptides/compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active peptides/compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the peptides/compound and a suitable powder base such as lactose or starch.

The compositions of the present invention can also be formulated in rectal compositions such as suppositories or retention enemas, e.g., containing conventional suppository bases such as cocoa butter or other glycerides.

In addition to the formulations described previously, the compositions of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the peptides/compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present invention, for example, can be applied to a plaster, or can be applied by transdermal, therapeutic systems that are consequently supplied to the organism.

The compositions of the present invention can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

This invention and embodiments illustrating the method and materials used may be further understood by reference to the following non-limiting examples. The present invention is described in connection with the following examples which are set forth for the purposes of illustration only. In the examples, optically active protected amino acids in the L-configuration are used except where specifically noted. The following Examples set forth suitable methods of synthesizing the novel GHRH agonists by the solid-phase technique.

### EXAMPLES

### Example 1

Synthesis of N-Me-Tyr¹-Ala²-Asp³-Ala⁴-Ile⁵-Fpa5⁶-Thr⁷-Gln⁸-Ser⁹-Tyr¹⁰-Arg¹¹-Orn¹²-Var³-Leu¹⁴-Abu¹⁵-Gln¹⁶-Leu¹⁷-Ser¹⁸-Ala¹⁹-A²⁰-Orn²¹-Leu²²-Leu²³-Glu²⁴-Asp²⁵-Ile²⁶-Nle²⁷-Asp²⁸-Agm²⁹ or [N-Me-Tyr¹, Fpa5⁶, Gin⁸, Orn¹², Abu¹⁵, Orn²¹, Nle²⁷, Asp²⁸, Agm²⁹]hGHRH(1-29) (Peptide 20103).

The synthesis is conducted in a stepwise manner using manual solid phase peptide synthesis equipment. Two methods have been used for the synthesis of peptides having Agm at the C-terminus. In one case, the starting material of the synthesis is Boc-agmatine-NG-sulfonyl-phenoxyacetyl-MBHA (Boc-Agm-SPA-MBHA) resin with a substitution of 0.3 mmol/g, which was obtained commercially from California Peptide Research, Inc. (Napa, Calif.). The synthesis of this resin is well known in the art. Briefly, Boc-Agm-SPA-MBHA resin (1.67 g, 0.50 mmol) is pre-swollen in DCM and then the deprotection and neutralization protocols are performed in order to remove the Boc protecting group and prepare the peptide-resin for coupling of the next amino acid. In another case, Agm-sulfonyl-polystyrene (PS) resin is used [1% DVB, 100-200 mesh, 0.74 mmol/g, American Peptide Company (Sunnyvale, Calif.)]. Briefly, Agm-sulfonyl-PS resin (680 mg, 0.50 mmol) is neutralized with 5% DIEA in DCM and washed according to the standard protocols. The solution of Boc-Asp(OcHx)-OH (475 mg, 1.5 mmol) in DMF-DCM (1:1) is shaken with the neutralized resin and DIC (235 μl, 1.5 mmol) in a manual solid phase peptide synthesis apparatus for 1 hour. Then, the deprotection and neutralization protocols are performed in order to remove the Boc protecting group and prepare the peptide-resin for coupling of the next amino acid. The synthesis is continued in a stepwise manner using manual solid phase peptide synthesis equipment in both cases, and the peptide chain is built stepwise by coupling the following protected amino acids in the indicated order on the resin to obtain the desired peptide sequence: Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Gln-OH, Boc-Thr(Bzl)-OH, Boc-Fpa5-OH, Boc-Ile-OH, Boc-Ala-OH, Boc-Asp(OcHx)-OH, Boc-Ala-OH, Boc-N-Me-Tyr(2BrZ)-OH.

These protected amino acid residues (also commonly available from NovaBiochem, Advanced Chemtech, Bachem, and Peptides International) are represented above according to a well accepted convention. The suitable protecting group for the side chain functional group of particular amino acids appears in parentheses. The OH groups in the above formulae indicate that the carboxyl terminus of each residue is free.

The protected amino acids (1.5 mmol each) are coupled with DIC (235 ul, 1.5 mmol) with the exceptions of Boc-Asn-OH and Boc-Gln-OH which are coupled with their preformed HOBt esters.

In order to cleave the peptide from the resin and deprotect it, a portion of 250 mg of the dried peptide resin is stirred with 0.5 mL m-cresol and 5 mL hydrogen fluoride (HF) at 0° C. for 2 hours. After evaporation of the HF under a stream of nitrogen and in vacuo, the residue is washed with dry diethyl ether and ethyl acetate. The cleaved and deprotected peptide is dissolved in 50% acetic acid and separated from the resin by filtration. After dilution with water and lyophilization, 68 mg crude product is obtained.

The crude peptide is checked by analytical HPLC using a Hewlett-Packard Model HP-1090 liquid chromatograph equipped with a Supelco Discovery HS C18 reversed-phase column (2.1 mm×5 cm, packed with C18 silica gel, 300 A pore size, 3 m particle size) (Supelco, Bellefonte, Pa.). Linear gradient elution (e.g., 40-70% B) is used with a solvent system consisting of (A) 0.1% aqueous TFA and (B) 0.1% TFA in 70% aqueous MeCN, and the flow rate is 0.2 mL/min. Purification is performed on a Beckman System Gold HPLC system (Beckman Coulter, Inc., Brea, Calif.) equipped with 127P solvent Module; UV-VIS Detector, model 166P; Computer workstation with CPU Monitor and printer, and 32-Karat software, version 3.0. 68 mg of crude peptide is dissolved in AcOH/H₂O, stirred, filtered and applied on an XBridge Prep OBD™ reversed phase column (4.6×250 mm, packed with C₁₈ silica gel, 300 A pore size, 5 m particle size) (Waters Co., Milford, Mass.). The column is eluted with a solvent system described above in a linear gradient mode (e.g., 40-60% B in 120 min); flow rate 12 mL/min. The eluent is monitored at 220 nm, and fractions are examined by analytical HPLC. Fractions with purity higher than 95% are pooled and lyophilized to give 18 mg pure product. The analytical HPLC is carried out on a Supelco Discovery C18 reversed-phase column described above using isocratic elution with a solvent system described above with a flow rate of 0.2 mL/min. The peaks are monitored at 220 and 280 nm. The product is judged to be substantially (>95%) pure by analytical HPLC. Molecular mass is checked by electrospray mass spectrometry, and the expected amino acid composition is confirmed by amino acid analysis.

In accordance with the above procedure Peptide 20105, Peptide 20107, Peptide 20109, Peptide 20110, Peptide 20111, Peptide 20113, Peptide 20115, Peptide 20350, Peptide 20351, Peptide 20356, Peptide 20357, Peptide 20358, Peptide 20359, Peptide 20360, Peptide 20361, Peptide 20363, Peptide 20367, Peptide 20370, Peptide 20371, Peptide 20372, Peptide 20373, Peptide 20374, Peptide 20375, Peptide 20376, are synthesized in the same manner as Peptide 20103, except that these peptides also contain other amino acid substitutions in the peptide sequence, and acyl moieties at their N-termini. The details for these syntheses are set forth below.

For the synthesis of Peptide 20105, the chemical structure of which [N-Me-Tyr¹, D-Ala², Fpa5⁶, Gln⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Gln-OH, Boc-Thr(Bzl)-OH, Boc-Fpa5-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20107, the chemical structure of which is [[N-Me-Tyr1, Fpa⁵⁶, Ala⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Ala-OH, Boc-Thr(Bzl)-OH, Boc-Fpa5-OH, Boc-Ile-OH, Boc-Ala-OH, Boc-Asp(OcHx)-OH, Boc-Ala-OH, Boc-N-Me-Tyr(2BrZ)-OH.

For the synthesis of Peptide 20109, the chemical structure of which is [N-Me-Tyr¹, D-Ala², Fpa5⁶, Ala⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Ala-OH, Boc-Thr(Bzl)-OH, Boc-Fpa5-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20110, the chemical structure of which is [N-Me-Tyr¹, D-Ala², Fpa5⁶, Orn¹², Abu¹⁵, Orn²¹, Nle²⁷, Agm²⁹]hGHRH(1-29), the following protected amino acids are coupled in the indicated order on the Agm-SO₂-PS resin: Boc-Ser(Bzl)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Asn-OH, Boc-Thr(Bzl)-OH, Boc-Fpa5-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20111, the chemical structure of which is [N-Me-Tyr¹, D-Ala², Fpa5⁶, Thr⁸, Orn¹², Abu¹⁵, Orn²¹, Nle²⁷, Asp²⁸, Agm²⁹]hGHRH(1-29), the following protected amino acids are coupled in the indicated order on the resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Thr(Bzl)-OH, Boc-Thr(Bzl)-OH, Boc-Fpa5-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20113, the chemical structure of which is [N-Me-Tyr¹, Fpa5⁶, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Asn-OH, Boc-Thr(Bzl)-OH, Boc-Fpa5-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20115, the chemical structure of which is [N-Me-Tyr¹, Fpa5⁶, Thr⁸, Orn¹², Abu¹⁵, Orn²¹, Nle²⁷, Asp²⁸, Agm²⁹]hGHRH(1-29) the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Thr(Bzl)-OH, Boc-Thr(Bzl)-OH, Boc-Fpa5-OH, Boc-Ile-OH, Boc-Ala-OH, Boc-Asp(OcHx)-OH, Boc-Ala-OH, Boc-N-Me-Tyr(2BrZ)-OH.

For the synthesis of Peptide 20117 the chemical structure of which [N-Me-Tyr¹, D-Ala², Fpa5⁶, Gln⁸, Orn¹², Abu¹⁵, Orn²¹, Nle²⁷, Agm²⁹]hGHRH(1-29), the following protected amino acids are coupled in the indicated order on the Agm-SO₂-PS resin: Boc-Ser(Bzl)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Gln-OH, Boc-Thr(Bzl)-OH, Boc-Fpa5-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20350 the chemical structure of which [Dat¹, D-Ala², Gin⁸, Orn¹², Abu¹⁵, Orn²¹, Nle²⁷, Asp²⁸, Agm²⁹]hGHRH(1-29), the following protected amino acids are coupled in the indicated order on the resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Gln-OH, Boc-Thr(Bzl)-OH, Boc-Fpa5-OH, Boc-Ile-OH, Boc-Ala-OH, Boc-Asp(OcHx)-OH, Boc-D-Ala-OH, Dat-OH.

For the synthesis of Peptide 20351 the chemical structure of which [Ac-N-Me-Tyr¹, D-Ala², Gln⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Gln-OH, Boc-Thr(Bzl)-OH, Boc-Fpa5-OH, Boc-Ile-OH, Boc-Ala-OH, Boc-Asp(OcHx)-OH, Boc-D-Ala-OH, Ac-N-Me-Tyr(2BrZ)-OH.

For the synthesis of Peptide 20356, the chemical structure of which [N-Me-Tyr¹, Gln⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Gln-OH, Boc-Thr(Bzl)-OH, Boc-Fpa5-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20357 the chemical structure of which [Dat¹, D-Ala², N-Me-Ala⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-N-Me-Ala-OH, Boc-Thr(Bzl)-OH, Boc-Fpa5-OH, Boc-Ile-OH, Boc-Ala-OH, Boc-Asp(OcHx)-OH, Boc-D-Ala-OH, Dat-OH.

For the synthesis of Peptide 20358 the chemical structure of which [Dat¹, D-Ala², N-Me-Ala⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-N-Me-Ala-OH, Boc-Thr(Bzl)-OH, Boc-Fpa5-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20359, the chemical structure of which is [N-Me-Tyr¹, D-Ala², Fpa5⁶, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Asn-OH, Boc-Thr(Bzl)-OH, Boc-Fpa5-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20360, the chemical structure of which is [N-Me-Tyr¹, D-Ala², Thr⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Thr(Bzl)-OH, Boc-Thr(Bzl)-OH, Boc-Phe-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20361, the chemical structure of which is [N-Me-Tyr¹, D-Ala², Gln⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Gln-OH, Boc-Thr(Bzl)-OH, Boc-Phe-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20367, the chemical structure of which is [N-Me-Tyr¹, D-Ala², Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Asn-OH, Boc-Thr(Bzl)-OH, Boc-Phe-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20370, the chemical structure of which is [N-Me-Tyr¹, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Asn-OH, Boc-Thr(Bzl)-OH, Boc-Phe-OH, Boc-Ile-OH, Boc-Ala-OH, Boc-Asp(OcHx)-OH, Boc-Ala-OH, Boc-N-Me-Tyr(2BrZ)-OH.

For the synthesis of Peptide 20371, the chemical structure of which is [N-Me-Tyr¹, Thr⁸, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Thr(Bzl)-OH, Boc-Thr(Bzl)-OH, Boc-Phe-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20372, the chemical structure of which is [N-Me-Tyr¹, Orn¹², Abu¹⁵, Orn²¹, Nle²⁷, Asp²⁸, Agm²⁹]hGHRH(1-29), the following protected amino acids are coupled in the indicated order on the Agm-SO₂-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Ala-OH, Boc-Thr(Bzl)-OH, Boc-Phe-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20373, the chemical structure of which is [N-Me-Tyr, Gln⁸, Orn², Abu¹⁵, Orn²¹, Nle²⁷, Agm²⁹]hGHRH(1-29), the following protected amino acids are coupled in the indicated order on the Agm-SO2-PS resin: Boc-Asp(OcHx)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Gln-OH, Boc-Thr(Bzl)-OH, Boc-Phe-OH, Boc-Ile-OH, Boc-Ala-OH, Boc-Asp(OcHx)-OH, Boc-D-Ala-OH, Boc-N-Me-Tyr(2BrZ)-OH.

For the synthesis of Peptide 20374, the chemical structure of which is [N-Me-Tyr¹, Orn¹², Abu¹⁵, Orn²¹, Nle²⁷, Agm²⁹]hGHRH(1-29), the following protected amino acids are coupled in the indicated order on the Agm-SO₂-PS resin: Boc-Ser(Bzl)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Asn-OH, Boc-Thr(Bzl)-OH, Boc-Phe-OH, Boc-Ile-OH, Boc-Ala-OH, Boc-Asp(OcHx)-OH, Boc-Ala-OH, Boc-N-Me-Tyr(2BrZ)-OH.

For the synthesis of Peptide 20375, the chemical structure of which is [N-Me-Tyr¹, Thr⁸, Orn¹², Abu¹⁵, Orn²¹, Nle²⁷, Agm²⁹]hGHRH(1-29), the following protected amino acids are coupled in the indicated order on the Agm-SO₂-PS resin: Boc-Ser(Bzl)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Thr(Bzl)-OH, Boc-Thr(Bzl)-OH, Boc-Phe-OH, Boc-Ile-OH, Boc-Ala-OH, Boc-Asp(OcHx)-OH, Boc-Ala-OH, Boc-N-Me-Tyr(2BrZ)-OH.

For the synthesis of Peptide 20376, the chemical structure of which is [N-Me-Tyr¹, Orn¹², Abu¹⁵, Orn²¹, Nle²⁷, Agm²⁹]hGHRH(1-29), the following protected amino acids are coupled in the indicated order on the Agm-SO₂-PS resin: Boc-Ser(Bzl)-OH, Boc-Nle-OH, Boc-Ile-OH, Boc-Asp(OcHx)-OH, Boc-Gln-OH, Boc-Leu-OH, Boc-Leu-OH, Boc-Orn(2ClZ)-OH, Boc-Arg(Tos)-OH, Boc-Ala-OH, Boc-Ser(Bzl)-OH, Boc-Leu-OH, Boc-Gln-OH, Boc-Abu-OH, Boc-Leu-OH, Boc-Val-OH, Boc-Orn(2ClZ)-OH, Boc-Arg(Tos)-OH, Boc-Tyr(2BrZ)-OH, Boc-Ser(Bzl)-OH, Boc-Ala-OH, Boc-Thr(Bzl)-OH, Boc-Phe-OH, Boc-Ile-OH, Boc-Ala-OH, Boc-Asp(OcHx)-OH, Boc-Ala-OH, Boc-N-Me-Tyr(2BrZ)-OH.

HF cleavage and deprotection, and subsequent purification by semipreparative HPLC of Peptide 20105, Peptide 20107, Peptide 20109, Peptide 20110, Peptide 20111, Peptide 20113, Peptide 20115, Peptide 20350, Peptide 20351, Peptide 20356, Peptide 357, Peptide 20358, Peptide 20359, Peptide 20360, Peptide 20361, Peptide 20363, Peptide 20367, Peptide 20370, Peptide 20371, Peptide 20372, Peptide 20373, Peptide 20374, Peptide 20375, Peptide 20376 are done as described in the case of Peptide 20103. The purified compounds are judged to be substantially (>95%) pure by analytical HPLC. Their molecular masses are checked by electrospray mass spectrometry, and the expected amino acid compositions are confirmed by amino acid analysis.

### Example 2

Combination of a chemotherapy agent and a GHRH agonist peptide inhibits glioblastomas.

Materials and Methods

Peptides and Chemicals. The GHRH agonist, JI-34, was synthesized by solid-phase method and purified by reversed-phase HPLC. The structure of JI-34 is [Dat¹, Orn¹², Abu¹⁵, Orn²¹, Nle²⁷, Asp²⁸, Agm²⁹]hGHRH-(1-29) (Abu, α-aminobutyric acid; Agm, agmatine; Dat, desaminotyrosine; Nle, norleucine; Orn, ornithine). For studies in vivo, we used daily s.c. injections of JI-34 dissolved in 0.1% DMSO (Sigma) in 10% (vol/vol) aqueous propylene glycol (vehicle solution). For in vitro experiments, JI-34 was dissolved in 0.1% DMSO and diluted with incubation medium. DOX (Chemtec Leuna) was administered i.v. and dissolved in 0.01 N acetic acid and diluted with 5% (wt/vol) mannitol.

Animal Experiments

Six-wk-old nude mice (Ncr nu/nu) were obtained from the National Cancer Institute (Bethesda, Md.). The animals were housed in sterile cages in a temperature-controlled room with a 12-h light/12-h dark schedule and were fed with autoclaved chow and water, ad libitum. The Institutional Animal Care and Use Committee, VA Medical Center Miami, Fla. fully approved the animal protocols. For the therapy study, 10⁶ cells per mouse of the human glioblastoma cell line, U-87 MG (American Type Culture Collection, ATCC), were injected into the flanks of four nude mice (Ncr nu/nu) under isoflurane anesthesia (Baxter). A month later, the resulting tumors were harvested and minced into ˜3 mm³ pieces and transplanted into flanks of nude mice, under anesthesia, using a minitrocar. When the tumors reached ˜32 mm³, the mice were randomized into four groups. The animals received the following treatments for 6 wk: (Group 1) control, 16 tumors, vehicle solution; (group 2) agonist JI-34, 1 μg/20 g, s.c., daily, 19 tumors; (group 3) DOX, 260 nmol/20 g, i.v., weekly on Thursdays, 19 tumors; and (group 4) JI-34 (1 μg/20 g daily)+DOX (260 nmol/20 g), 16 tumors. Tumor volume was measured with microcalipers once a week and calculated using the formula: (length×width×height×in)/6. Tumor doubling time was calculated using the formula: [study duration×logarithm (LOG) 2]/(LOG final tumor volume−LOG initial tumor volume). At the end of the experiment, the mice were killed under pentobarbital anesthesia, tumors were excised and weighed; necropsy was performed. Samples free of necrotic debris were immediately snap frozen in liquid nitrogen and stored at −80° C. for further analyses.

Cell Maintenance and Cell Counting

U-87 MG cells were cultured in Eagle's minimum essential medium (ATCC) medium [supplemented with 10% FBS (ATCC) and 0.1% penicillin/streptomycin] at 37° C. and 5% CO2 atmosphere. For cell count and cell size determination, 103 cells were seeded into T-25 flasks. The medium was changed two to three times a week. The following treatment in vitro groups were set up: (Group 1) control, vehicle solution; (group 2) JI-34 at 1 μM final concentration, daily; (group 3) DOX at 100 nM final concentration, weekly on Thursdays; and (group 4) JI-34, 1 μM and DOX, 100 nM. After 3 wk, cell count and cell size were determined by Z Series Coulter Counter (Beckman Coulter).

Cell Volume Determination

Cell volume was estimated by measuring the intracellular water space as follows. Briefly, 1 mM 3-O-methylglucose (3-OMG) and 0.5 μCi/mL [3H]-3-OMG were added to the culture 6 h before the volume assay. At the end of the incubation period, culture medium was aspirated, and an aliquot was saved for radioactivity determination. Cells were washed rapidly six times with ice-cold buffer containing 229 mM sucrose, 1 mM Trisnitrate, 0.5 mM calcium nitrate, and 0.1 mM phloretin, pH 7.4. Cells were harvested into 0.5 mL of 1 N sodium hydroxide. Radioactivity in the cell extracts and media was determined, and an aliquot of the cell extract was used for protein estimation with the Bio-Rad bicinchoninic acid kit. Values were normalized to protein level, and cell volume was expressed as microliters/milligram protein.

Proliferation Assays In Vitro

For proliferation studies, 104 cells per well were seeded in 100 μL media, in a 96-well plate, and incubated for 24 h in a humidified incubator at 37° C. Then, culture medium was replaced with FBSfree medium (starvation) for 24 h. After 24 h, the cells received full medium containing the following treatments: Groups 1 (control) and 3, vehicle solution; groups 2 and 4, JI-34 (1 μM final concentration). After another 24 h, the cells received the following treatments: group 1, vehicle solution; group 2, JI-34; group 3, DOX (100 nM final concentration); and group 4, JI-34+DOX. Then, they were incubated for 48 h and the effect on cell proliferation was evaluated by using the MTT assay (CellTiter 96 Non-Radioactive Cell Proliferation Assay; Promega), according to the manufacturer's instructions with the help of a Victor3 multilabel counter (Perkin-Elmer).

Apoptosis Assay

Determinations of viability and apoptosis were performed from freshly seeded cell samples (104 cells per well, in 100 μL media, in a 96-well plate) with the help of ApoLive-Glo Multiplex Assay (Promega), according to the manufacturer's instructions. First the cells received full medium containing the following treatments: Groups 1 (control) and 3, vehicle solution; groups 2 and 4, JI-34 (1 μM final concentration). After 24 h, the cells received the following treatments: group 1, (vehicle solution); group 2, JI-34; group 3, DOX (100 nM final concentration); and group 4, JI-34+DOX. After another 24 h of incubation, viability reagent was added to the wells and fluorescence was measured by Victor3 multilabel counter. Then Caspase-Glo 3/7 apoptosis reagent was added and luminescence was determined.

Immunocytochemistry

Cells were seeded onto coverslips in six-well plates (50,000 cells per well) in 10% FBS-containing growth medium. This medium was replaced with serum-free medium the following day for 24 h. Thereafter, GHRH agonist (JI-34, 1 μM) was added in medium supplemented with 0.1% FBS and remained for the consecutive 2 d. Control cells received vehicle (DMSO). At the end of the treatment, phase-contrast images of live cells were collected and cells were fixed in ice-cold acetone for 10 min, washed with PBS three times, and blocked with 2% goat serum in PBS for 30 min. GFAP (Abcam; 1:500 dilution) or nestin (BD Transduction Laboratories; 1:75 dilution) antibodies were added in PBS for 1 h. Anti-rabbit and anti-mouse secondary antibodies (Alexa Fluor 488; Jackson Immunoresearch) were also applied for 1 h. Coverslips were mounted in Vectashield mounting medium containing DAPI for nuclear staining (Vector Laboratories). Images were acquired on a Nikon Eclipse Ti fluorescence microscope (Nikon Instruments).

MDR Assay

The multidrug resistance assays were performed according to the manufacturer's instructions (Cayman Chemical). U-87 MG cells were seeded, 5×104 cells per well density in 100 μL medium in 96-well, black, clear-bottom plates and grown overnight in a humidified incubator at 37° C. The next day the medium was discarded, and the cells were treated according to the following protocol: Groups 1 (control) and 3, vehicle solution; groups 2 and 4, JI-34 (1 μM final concentration). After another 24 h, the cells received the following treatments: Group 1, vehicle solution; group 2, JI-34; group 3, DOX (100 nM final concentration); and group 4, JI-34+DOX. During the second treatment, half of the control wells were treated with cyclosporin-A solution in 1/1,000 dilution as positive control according to the manufacturer's description. Afterward, the cells were incubated for 1 h, then calcein AM/Hoechst dye combined staining solution was added. Fifteen minutes later, both cell density (at excitation and emission wavelengths of 355 nm and 465 nm, respectively) and calcein retention (at excitation and emission wavelengths of 485 nm and 535 nm, respectively) were detected with the help of a Victor3 multilabel counter. Relative calcein retention values were expressed as a function of cell density.

Total RNA Isolation and Reverse Transcription

Total RNA was isolated from representative, DNase treated, U-87 MG dissected tumor samples using a NucleoSpin kit according to the manufacturer's instructions (Macherey-Nagel). Four tumor samples from each group were analyzed. The yield and the quality of RNA samples were determined spectrophotometrically using 260 nm, and 260/280- and 260/230-nm ratio. The synthesis of cDNA was performed as follows. Briefly, 1 μg of RNA from each sample was reverse transcribed into cDNA by a RT First Strand kit (Qiagen). Reverse transcription was done in a Veriti 96-well thermal cycler (Applied Biosystems).

Cancer Pathway Finder Quantitative PCR Array

The Human Cancer PathwayFinder quantitative PCR array (PAHS-033A; Qiagen) used in our study contains 84 unique genes related to cell proliferation, apoptosis, cell cycle, angiogenesis, invasion, and metastasis. All PCR arrays were performed using the iQ5 Multicolor Real-Time Detections system (Bio-Rad). All genes represented by the array showed a single peak on the melting curve characteristic of the specific products. Experiments were run in triplicate for each study group. Analysis of gene expression data was performed using Excel-based PCR Array Data Analysis software provided by the manufacturer (Qiagen). Fold changes in gene expression were calculated using the ΔΔCt method and five stably expressed housekeeping genes (B2M, HPRT1, RPL13A, GAPDH, and ACTB) were used for normalization of the results.

ELISA

Glioblastoma cells (10⁵ cells per well) were seeded onto six-well plates, cultured overnight, and then exposed to compounds used in the 3-(4,5-dimethylthiazol-2-yl)-2,5-diphenyltetrazolium bromide proliferation assays: Groups 1 (control) and 3, vehicle solution; groups 2 and 4, GHRH agonists JI-34 (1 μM final concentration). After another 24 h, the cells received the following treatments: Group 1, vehicle solution; group 2, JI-34; group 3, doxorubicin (DOX) (100 nM final concentration); and group 4, JI-34+DOX. Concentrations of the specific proteins in the medium (in the case of FGF basic or TGFβ-1) or in samples of scraped and homogenized cells (in the case of p53) were determined after 24 h using ELISA kits according to the manufacturer's instructions. Human TGFβ and FGF basic ELISA kits were obtained from AbCam, whereas p53 was measured with a PathScan Sandwich ELISA kit (Cell Signaling Technology). Readings were normalized to protein concentrations as determined by NanoDrop (NanoDrop Technologies).

Western Blot Analyses

Protein from the tumor tissue was isolated using the NucleoSpin kit (Macherey-Nagel). Protein concentrations were determined by NanoDrop (NanoDrop Technologies). Equal amounts of protein were resuspended in sample loading buffer (0.25 M Trizma Base, 8% SDS, 40% glycerol, 0.004% bromophenol blue, 4% P3-mercaptoethanol; pH 6.8), boiled for 3 min and separated by 12% SDS-polyacrylamide gel electrophoresis. Proteins from the gel were then transferred onto nitrocellulose membranes, which were blocked with 50-50% Tris-buffered saline (20 mM Tris.HCl pH 7.5, 150 mM NaCl) and Odyssey blocking buffer for 1 h at room temperature, followed by an overnight incubation at 4° C. with the following primary antibodies: growth hormone-releasing hormone receptor (GHRH-R), (ab28692) nestin (ab92391), glial fibrillary acid protein (GFAP) (ab48050) (all from AbCam), β-actin (A5441; Sigma-Aldrich), or p53 (9282; Cell Signaling Technology). The GHRH-R antibody is targeted against the polypeptide segment found in both pituitary (p)GHRH and splice variant-1 (SV1) receptors. The signals were developed by incubating the nitrocellulose membrane for 1 h at room temperature with the appropriate Infrared IRDye-labeled secondary antibodies (1:10000; LI-COR Biosciences) and were then visualized with the Odyssey Infrared Imaging system (LI-COR Biosciences). The protein bands were quantified using V3.0 software (LI-COR Biosciences); integrated density values of triplicate experiments were plotted.

Statistical Analyses

Statistical analyses were performed using either t test for independent samples, univariate analysis of variance (ANOVA), or repeated measure ANOVA (RMANOVA). ANOVA was followed by Tukey's or Fisher's post hoc test, while RMANOVA was followed by Fisher's post hoc test. Results are expressed as the means±SEM. Differences, compared with the control, with P<0.05 considered as statistically significant. Statistical analyses and data reductions were performed by SigmaPlot 11.0 (Systat software) and IBM SPSS Statistics 20.0.

Results

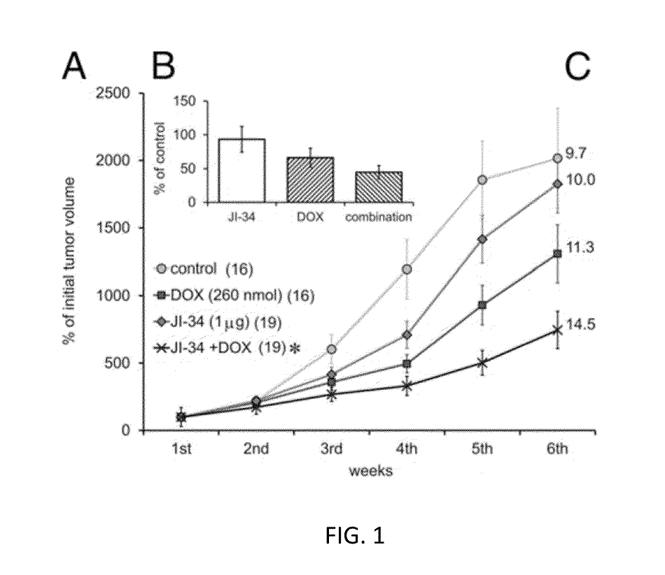

In vivo effects of the GHRH agonist JI-34 alone or in combination with doxorubicin (DOX) on the growth of glioblastoma U-87 MG xenografts were tested in nude mice. Nude mice bearing U-87 MG tumors were treated with the GHRH agonist, JI-34 (50 μg/kg/d), DOX (13 μmol/kg/wk), or with their combination for 6 wk. Control animals received daily saline injections. The administration of DOX reduced growth of U-87 MG tumors by 35%. The combination of JI-34 with DOX elicited an even greater (63%) inhibition of tumor growth (FIG. 1A) and repeated measure ANOVA revealed a significant difference (F_{3,66}=1.962, P<0.01, Fisher's post hoc test: P<0.05 between J-34+DOX and control). Similar suppressive effects of these therapies were observed on the basis of the reduction of final tumor weights at sacrifice (FIG. 1B) and increases in tumor doubling times (FIG. 1C).

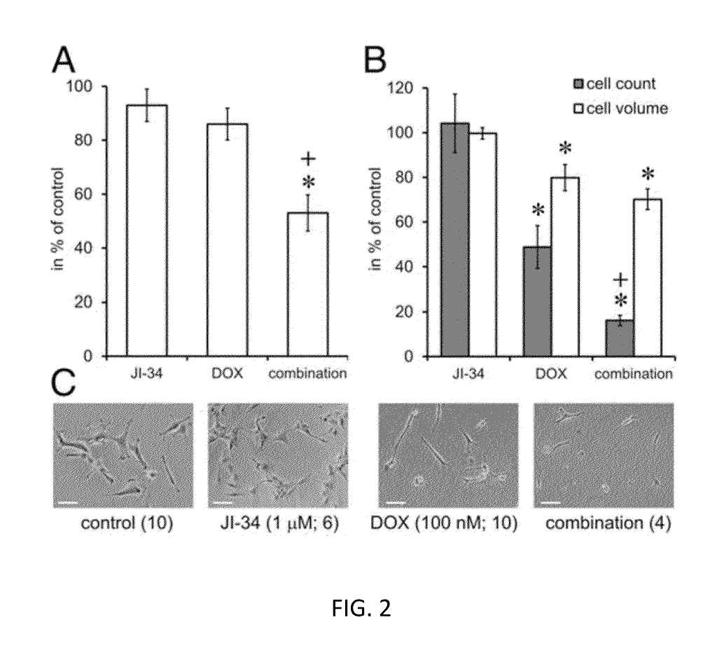

The in vitro effect of JI-34, DOX, or their combination on the proliferation rate of U-87 MG cells was tested using the 3-(4,5-dimethylthiazol-2-yl)-2,5-diphenyltetrazolium bromide (MTT) assay. Exposure to GHRH agonist, JI-34, alone at 1 μM concentration or 100 nM DOX alone as separate drugs, had no significant effect on the proliferation of U-87 MG cells in vitro. When cells were pretreated with 1 μM JI-34 for 24 h before exposure to the combination of DOX+JI-34 for 48 h, the inhibitory effect (47% inhibition) of the combination treatment proved to be statistically significant (FIG. 2A) (ANOVA; F_{3,20}=11.37, Tukey's post hoc: P<0.01 vs. both control and DOX).

In a different experiment, cells were cultured in T-25 flasks, and drugs were applied repeatedly (1 μM JI-34 daily and DOX weekly at 100 nM) for 3 wk (FIG. 2B). Cell number and cell volume were then determined by cell counting and cell volumetric assays, respectively. JI-34 alone had no significant effect, but both DOX and its combination with JI-34 significantly decreased the number of cells (52% and 84% decrease, respectively; F_{3,26}=19.51 and Tukey's post hoc: P<0.01 for DOX vs. control and combination vs. control, whereas Fisher post hoc: P<0.05 for combination vs. DOX). DOX and its combination with JI-34 also reduced the volume of the cells (20% and 30% decrease, respectively; F_{3,26}=9.87 and Tukey's post hoc: P<0.01 for DOX vs. control and combination vs. control) as assessed by intracellular water content. FIG. 2C shows representative microscopic images of all four treatment groups. The treatment with GHRH agonist resulted in adherent cultures of neuroectodermal cells with more prominent glial projections. Conversely, DOX treatment elicited characteristic fusiform changes in morphology due to arrested mitoses and the mitotic collapse that precedes apoptosis. The combination exerted a devastative effect, perhaps due to the mitotic synchronizing and sensitizing activity of JI-34.

The effect of GHRH agonist JI-34 and its combination with DOX on the expression of genes related to cell proliferation, apoptosis, cell cycle, angiogenesis, invasion, and metastasis were measured by RT-PCR. Quantitative realtime PCR array studies revealed a significant effect, of the combination of JI-34 and DOX treatment for 6 wk, on the expression of several markers of tumor growth, invasion, and metastasis formation in U-87 cell-derived tumors (Table 2). The combination of JI-34 and DOX increased the expression of the proapoptotic BCL-2-associated agonist of cell death (BAD) and decreased the expression of the anti-apoptotic B-cell lymphoma 2 (Bcl-2). The increase in BAD seems to be of special importance as DOX itself had an opposite effect on BAD expression. The combination treatment also attenuated the expression of the contact activator integrin α3 subunit (by approximately twofold). In addition, an almost threefold decrease was observed in the metastasis promoting S100 calcium binding protein A4 (S100-A4).

TABLE 2
Relative expression of genes related to tumor growth

| Treatment genes | DOX | JI-34 + DOX |
|---|---|---|
| BCL-2-associated agonist gas of cell death (BAD) | −1.1* | −1.11* |
| B-cell CLLllymphoma 2 (Bcl-2) | −1.8* | −1.86* |
| Integrin α3 (CD49C) | −1.65* | −1.85* |
| S100 calcium binding protein A4 (S100-A4) | −2.34* | −2.75* |

Three parallel experiments were run for both groups, *P<0.05 vs. control

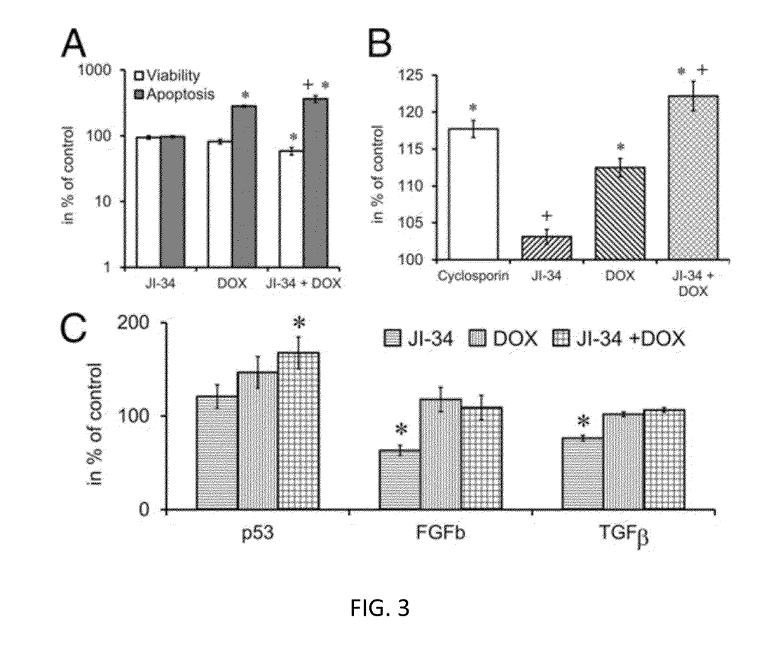

The molecular mechanisms underlying the combinatorial effect of JI-34 and DOX were analyzed. GHRH agonist JI-34 alone (1 μM) had no effect on apoptosis, but DOX at the 100-nM concentration and the combination of DOX 100 nM and JI-34 1 μM elicited a significant increase (182% and 263%, respectively) in apoptosis as measured by the ApoLive-Glo Multiplex assay after 24 h of treatment (FIG. 3A; F_{3,17}=41.31; Tukey's P<0.01 for DOX vs. control and combination vs. control and Fisher's P<0.05 combination vs. DOX). The combination of the DOX and JI-34 treatment also decreased the viability of the cells by 41% (F_{3,17}=7.00, Tukey's P<0.01 vs. control and Fisher's P<0.05 vs. DOX). In the multidrug resistance (MDR) assay both cyclosporin-A (manufacturer's recommended control) and DOX treatment increased calcein retention (17% and 7%, respectively; FIG. 3B; F_{4,86}=28.856, P<0.01; Tukey's post hoc test: P<0.01 vs. control). The JI-34 pretreatment significantly augmented calcein retention in the combination group by 23% compared with the control (F_{4,86}=28.856, P<0.01; Tukey's post hoc test: P<0.01 vs. control and P<0.01 vs. DOX). It appears that JI-34 pretreatment leads to decreased expression of MDR transporter proteins and increased calcein and DOX retention.

The effects of GHRH agonist JI-34, DOX, and their combination on the levels of intracellular p53 or secreted FGFb and TGFβ were analyzed. The changes in the expression of these regulators of tumor growth and differentiation in vitro were detected by ELISA experiments from either cell culture supernatants or from homogenates of U-87 cells treated with 100 nM DOX, 1 μM JI-34, or their combination (FIG. 3C). Using homogenized U-87 MG cell culture samples, a significant (68%) increase in the level of the tumor suppressor p53 was detected upon exposure of the cells to the treatment with combination of JI-34+DOX (F_{3,12}=3.6; Tukey's P<0.05 vs. control). The levels of both the glial growth factor, bFGF (F_{3,16}=5.15; Fisher's P<0.05 JI-34 vs. control), and the dedifferentiation and tumor promoting factor TGFβ (F_{3,10}=4.5; Fisher's P<0.05 JI-34 vs. control), were significantly lower (37% and 24%, respectively) in the supernatant of the cell cultures exposed to JI-34 treatment, demonstrating the inhibitory effects of JI-34 on the production of these cytokines.

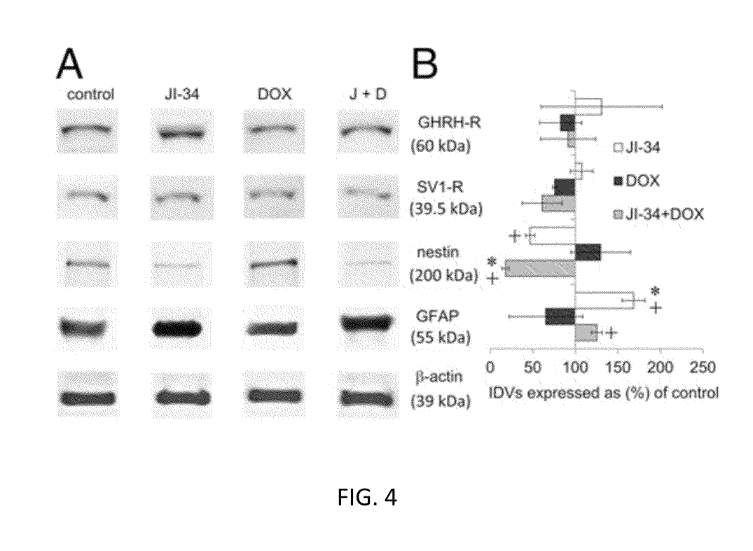

The effects of GHRH agonist JI-34 and DOX, alone and in combination, on the expression of GHRH Receptors, nestin and glial fibrillary acidic protein were analyzed. Western blot studies (FIG. 4) verified the expression of pituitary type GHRH receptor (pGHRH-R) and its splice variant, SV1, in samples of U-87 MG xenografts. None of the treatments significantly altered the expression of these receptors. The GHRH agonist decreased and the treatment with DOX increased the expression of the neuroectodermal stem-cell marker, nestin, but the effects on the maturation antigen glial fibrillary acid protein (GFAP) were opposite. In the case of both intermediary filaments, statistically significant differences could be observed according to the integrated density values. The combination treatment produced an 80% decrease in nestin expression compared with the control (F_{3,8=}7.991; Tukey's P<0.01 for JI-34+DOX vs. DOX, P<0.05 for JI-34+DOX vs. control and Tukey's P<0.05 for JI-34 vs. DOX), whereas the JI-34 pretreatment caused a 70% increase in GFAP expression compared with the control (F_{3,8}=7.991; Fisher's P<0.01 for JI-34 vs. DOX, P<0.05 for JI-34 vs. control and Fisher's P<0.05 for JI-34+DOX vs. DOX).

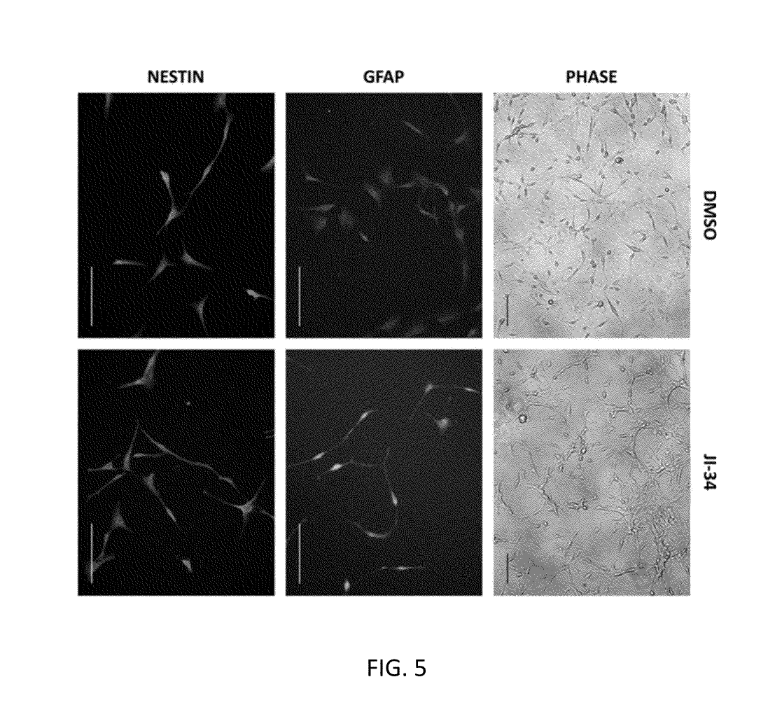

When U-87 MG cells were treated with the GHRH agonist JI-34 (1 M) in reduced serum-containing growth medium, cells underwent morphological changes, having more/longer astrocytic processes than cells in the control group. Representative phase-contrast images from control and GHRH agonist-treated groups are shown in FIG. 5. To further demonstrate the change in differentiation state following the administration of GHRH agonist, cells were fixed and stained for the detection of the astrocyte marker, GFAP, and the neuroectodermal stem cell marker, nestin. The elevation in the intensity of GFAP-labeling indicates that the GHRH agonist-treated cells attained a higher differentiation level than the untreated cells. The fluorescence staining with nestin antibody, however, did not show a significant change in the level of nestin following the administration of GHRH agonist.

### DISCUSSION

These results demonstrate that the concurrent administration of an agonistic analog of GHRH and a traditional cytotoxic drug, DOX, augments the antineoplastic action of the latter. These findings show that the treatment with the combination of the GHRH agonist and DOX inhibits the in vivo growth of xenotransplanted U-87 MG tumors as well as decreasing the multiplication and growth of these glioblastoma cells in vitro. This effect may be attributed to the ability of the GHRH agonist to induce changes in maturation state consequently decreasing the pluripotency of the neoplastic cells. Traditional cancer therapies frequently fail because of a phenomenon which may be called “survival of the fittest”, based upon the natural selection of cancer cells under evolutionary pressure exerted by the treatment itself, and which is analogous to the development of bacterial antimicrobial resistance under antibiotic therapy. This phenomenon may imply the presence of “cancer stem cells”. These cancer stem cells can provide an inexhaustible pool of cellular adaptation upon challenge, when there is no time for dedifferentiation, and the induction of the expression of resistance genes.

Based on present findings, it can be postulated that JI-34 sensitizing activity may be related to a maturation effect on the glioblastoma cells, a process characterized by the down-regulation of levels of nestin (a common neuroectodermal marker), and the up-regulation of levels of GFAP. Whereas DOX elicited relative increases in nestin and decrease in expression of GFAP, GHRH agonist JI-34 elicited opposite changes in the expression pattern of intermediary filaments in the present study.

Abrogation of cancer stem cells would be an initial step, a very special one, leading to the successful treatment of GBM and other malignancies by decreasing the resilience of the tumor and its ability to generate recurrence. Facilitation of maturation has already been demonstrated to be effective in the treatment of certain malignancies: destruction of the nonsense fusion protein denoted PML-RARα with arsenic-trioxide (AsO₃); liberation of RARα; administration of the strong maturation factor, retinoic acid, brings about long-lasting regression in acute promyelocytic leukemia. Retinoic acid is required for the differentiation of many epithelial cells. In addition, retinoids, as a sole treatment of tumor phenotypes with bioavailable receptors, have proven efficacy in basal cell carcinoma and Verruca vulgaris. Retinoids play an important role in the maturation of glial cells and have already been successfully used in the therapy of gliomas.

The down-regulation of GHRH receptors by potent GHRH agonist, which could lead to the development of resistance to the treatment, may be excluded as indicated by Western blot studies. This finding supports the view that GHRH analogs may manifest a broad range of activities and their pharmacologic profile cannot be limited to the down-regulation of their receptors, as occurs in prostate tumors and contributes to the well-known therapeutic effect of pharmacological castration.

The combination treatment of GHRH agonist and DOX also stimulated apoptosis and decreased the viability of the otherwise immortal U-87 cells in vitro. Activation of programmed cell death may be related to several initiating events, such as the synthesis of the most important tumor suppressor p53, a protein whose gene is often mutated in GBMs. Furthermore, a decrease in the expressions of the anti-apoptotic Bcl-2 and increase in the proapoptotic BAD gene were observed. The impact of the combination on BAD, which triggers apoptosis by inactivating Bcl-2 and Bcl-xL, was completely opposite to the effect of DOX alone.

Moreover, in sharp contrast with DOX, JI-34 elicited a downregulation of two important glial growth factors, FGF basic and TGFβ. This may also reflect stem cell maturation and a tendency to increased cellular mortality, because growth promoting cytokines are able to stimulate the survival cascades. The decrease in TGFβ is especially important, because this cytokine not only stimulates malignant transformation and growth, but also promotes angiogenesis, epithelial-mesenchymal transition (EMT), invasion, and suppresses peritumoral immune responses. Further, deprivation of growth factors may suspend the constitutive stimulatory activity of extracellular signal-regulated kinases/mitogen activated protein kinases (ERK/MAPKs) over Bcl-2. The combination significantly mitigated the transcription of the contact activator integrin domain, integrin α3 subunit. This molecule, as are other integrins, is frequently overexpressed in GBM tumors and plays an important role in the migration of neuroectodermal cells in both physiological and pathological circumstances. Upon binding laminin, fibronectin, or vitronectin, the integrin facilitates cell proliferation, EMT, and invasion through the activation of integrin-linked and focal adhesion kinases (FAKs). The down-regulation of the transcription of S100 calcium binding protein A4 (S100-A4) gene, which regulates microtubule polymerization and migration, may also contribute to the inhibition of cancer cell motility and metastatic spread. Moreover, S100-A4, beside activating motility, plays additional roles in proteolysis, EMT, angiogenesis, and cell survival.

Our demonstration that a change in the differentiation state of U-87 MG cells triggered by a GHRH agonist highly increases the susceptibility to cytotoxic treatments is particularly important for determining future strategies in the treatment of glioblastoma. GHRH analogs are promising candidates in this respect, as they readily bypass the blood-brain barrier. These findings suggest the potential for the development of new therapeutic paradigms in the treatment of brain cancers using agonistic as well as antagonistic analogs of hypothalamic GHRH. These GHRH analogs may augment the direct cytotoxic effect of traditional chemotherapeutic drugs, by stimulation and maturation of stem cells and eliciting cellular synchronization of the cell cycle.

What is claimed is:

1. A method of treating a subject with cancer comprising administering a therapeutically effective amount of at least one GHRH agonist peptide in combination with at least one anticancer agent.

2. The method of claim 1, wherein the at least one GHRH agonist peptide has the formula I:
[R₁-A¹-A²-A³-Ala-Ile-A⁶-A⁷-A⁸-A⁹-A¹⁰-A¹¹-A¹²-A¹³-A¹⁴-A¹⁵-A¹⁶-A¹⁷-A¹⁸-Ala-A²⁰-A²¹-A²²-A²³-A²⁴-A²⁵-A²⁶-A²⁷-A²⁸-A²⁹-A³⁰]-R₂;
wherein,
R₁ is Ac, Tfa, alkyl, cycloalkyl, benzyl, phenyl, substituted alkyl, substituted phenyl, substituted phenyl ethyl, 2-carboxybenzamido, carboxypropyl, amino-(hydroxylphenyl)-alkyl, amino(imidazole-4-yl)-alkyl, hydroxyphenyl-alkyl, or is absent;
A¹ is Tyr, Dat, des-amino Tyr, D-Tyr, Met, Phe, D-Phe, pCl-Phe, Leu, His, D-His, or N-Me-Tyr;
A² is Ala, D-Ala, Abu, D-Arg, Aib, D-N-methyl Ala, or D-Abu;
A³ is Asp, D-Asp, Glu, or D-Glu;
A⁶ is Phe, or Fpa5;
A⁷ is Thr, Aib, Leu, Trp, β-Nal, or pX-Phe, where X is H, F, Cl, Br, NO₂, or Me;
A⁸ is D-Asn, Asn, Ala, Gln, Thr, N-Me-Ala, Aib, Leu, Trp, β-Nal, Ser, D-Ser, or pX-Phe, wherein X is H, F, Cl, Br, NO₂, or Me;
A⁹ is Ser, Ala, Aib, Leu, Trp, β-Nal, or pX-Phe, where X is H, F, Cl, Br, NO₂, or Me;
A¹⁰ is Tyr or D-Tyr;
A¹¹ is Arg, His, Gap, Gab, or Har;
A¹² is Orn, Arg, Lys, D-Lys, Lys(Me)₂, Gap, Gab, N-alkyl-Lys, or N-cycloalkyl-Lys;
A¹³ is Val or Ile;
A¹⁴ is Leu or D-Leu;
A¹⁵ is Abu, Gly, Leu, Asn, Gln, Aib, D-Ala, or Ala;
A¹⁶ is Gln, Ala, or Aib;
A¹⁷ is Leu or D-Leu;
A¹⁸ is Ser or Tyr;
A²⁰ is Arg, His, Gap, Gab, or Har;
A²¹ is Orn, Lys, D-Lys, Arg, D-Arg, Gap, Gab, or Lys(Me)₂;
A²² is Leu, Ala, Abu, Lys, or Orn;
A²³ is Leu, D-Leu, Ala, or Abu;
A²⁴ is Gln, His, Ala, or Aib;
A²⁵ is Asp, Glu, D-Glu, D-Asp, Ala, or Aib;
A²⁶ is Ile or Leu;
A²⁷ is Nle, Met, D-Met, Ala, Ile, Leu, Nva, or Val;
A²⁸ is Ser, Asn, or Asp;
A²⁹ is Arg, Har, Agm, D-Arg, D-Har, or D-Har;
A³⁰ is Arg, Agm, Ada, Amc, Aha, Apa, Har, D-Arg, D-Har, Gab, Gln, D-Gln, Gln-Gab, D-Gln-Gab, or is absent; and
R₂ is —NH₂, —NH—CH₃, —NH—CH₂—CH₃, —OH, —CH₂)ₚ—NH—C(NH₂)═NH, —NH(CH₂)ₚ—C(═O)—NH₂, —NHR₃, —N(R₃)₂, or —OR₃, where p is an integer from 1 to 15, and R₃ is any of C₁₋₁₂ alkyl, C₂₋₁₂ alkenyl, or C₂₋₁₂ alkynyl, and pharmaceutically acceptable salts thereof, and
wherein the agonist peptide is different from SEQ ID NO: 1 in at least one amino acid residue.

3. The method of claim 1, wherein the at least one GHRH agonist peptide has the formula II:
[R₁-A¹-A²-Asp-Ala-Ile-A⁶-Thr-A⁸-Ser-Tyr-A¹¹-A¹²-Val-Leu-A¹⁵-Gln-Leu-Ser-Ala-A²⁰-A²¹-A²²-Leu-Gln-Asp-Ala-Ile-Nle²⁷-A²⁸-A²⁹-A³⁰]-R₂,
wherein
R¹ is Ac, Tfa, or is absent,
A¹ is Tyr, Dat, or N-Me-Tyr,
A² is Ala, D-Ala, Abu, or D-Abu,
A⁶ is Phe or Fpa5,
A⁸ is Asn, Ala, Gln, Thr, or N-Me-Ala,
A¹¹ is Arg, His, or Har,
A¹² is Orn, or Lys(Me)₂,
A¹⁵ is Abu or Ala,
A²⁰ is Arg, His, or Har,
A²¹ is Orn, or Lys(Me)₂,
A²² is Leu, or Orn,
A²⁸ is Ser, or Asp,
A²⁹ is Arg, Har, Agm, D-Arg, or D-Har,
A³⁰ is Arg, Agm, Ada, Amc, Aha, Apa, Har, D-Arg, D-Har, Gab, Gln, D-Gln, Gln-Gab, D-Gln-Gab, or is absent,
R₂ is —NH₂, —NH—CH₃, NH—CH₂—CH₃, —NH—CH₃, —OH, —NHR₃, —N(R₃)₂, or —OR₃, wherein R₃ is any of C₁₋₁₂ alkyl, C₂₋₁₂ alkenyl, or C₂₋₁₂ alkynyl,
and pharmaceutically acceptable salts thereof, and wherein the agonist peptide is different from SEQ ID NO: 1 in at least one amino acid residue.

4. The method of claim 1, wherein the at least one GHRH agonist peptide is of formula III:

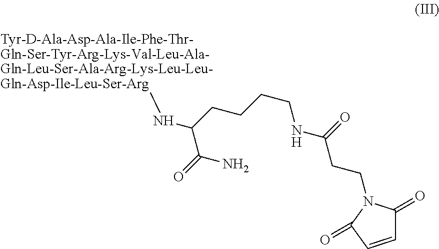

5. The method of claim 1, wherein the at least one GHRH agonist peptide is of formula IV:
Z¹-Z²-Asp-Ala-Ile-Phe-The-Asn-Ser-Tyr-Arg-Lys-Val-Leu-Z³-Gln-Leu-Ser-Ala-Arg-Lys-Leu-Leu-Gln-Asp-Ile-Z⁴-Z⁵-Arg-Gln-Gln-Gly-Glu-Z⁶-Asn-Gln-Glu-Z-Gly-Ala-OH, (IV)
wherein
Z¹ is a D- or L-isomer of the aminoacid tyrosine or histidine;
Z² is a D- or L-isomer of the aminoacid alanine, valine, or isoleucine;
Z³ is a D- or L-isomer of the aminoacid alanine or glycine;
Z⁴ is a D- or L-isomer of the aminoacid methionine or leucine;
Z⁵ is a D- or L-isomer of the aminoacid serine or asparagine;
Z⁶ is a D- or L-isomer of the aminoacid arginine or serine; and
Z⁷ is a D- or L-isomer of the aminoacid glutamine or arginine.

6. The method of claim 1, wherein the at least one GHRH agonist peptide is a stapled GHRH agonist peptide of formula V.

7. The method of claim 1, wherein the at least one GHRH agonist peptide is tesamorelin.

8. The method of claim 1, wherein the at least one GHRH peptide is selected from GHRH (1-30) represented by SEQ ID NO: 1, GHRH(1-44) represented by SEQ ID NO: 43, AKL-0707, and any combination thereof.

9. The method of claim 1, wherein the cancer is selected from colorectal cancer, breast cancer, ovarian cancer, pancreatic cancer, head and neck cancer, bladder cancer, liver cancer, renal cancer, melanoma, gastrointestinal cancer, prostate cancer, small cell and non-small cell lung cancer, sarcomas, glioblastoma, T- and B-cell lymphoma, endometrial cancer, and cervical cancer.

10. The method of claim 1, wherein the administration of the at least one GHRH agonist peptide is by parenteral administration selected from the group consisting of subcutaneous, intramuscular, intraperitoneal, intracavity, intrathecal, transdermal and intravenous injection.

11. The method of claim 1, wherein the GHRH agonist peptide is selected from:
P-20103 [N-Me-Tyr¹, Fpa5⁶, Gln⁸, Orn¹², Abu¹⁵, Orn²¹, Nle²⁷, Asp²⁸, Agm²⁹]hGHRH(1-29)NH₂(SEQ ID NO: 2);
P-20105 [N-Me-Tyr¹, D-Ala², Fpa5⁶, Gln⁸, Orn¹², Abu¹⁵, Orn²¹, Nle²⁷, Asp²⁸, Agm²⁹]hGHRH(1-29)NH₂;
P-20107 [N-Me-Tyr¹, Fpa5⁶, Ala⁸, Orn¹², Abu¹⁵, Orn²¹, Nle²⁷, Asp²⁸, Agm²⁹]hGHRH(1-29)NH₂(SEQ ID NO: 3);
P-20109 [N-Me-Tyr¹, D-Ala², Fpa5⁶, Ala⁸, Orn¹², Abu¹⁵, Orn²¹, Nle²⁷, Asp²⁸, Agm²⁹]hGHRH(1-29)NH₂;
P-20110 [N-Me-Tyr¹, D-Ala², Fpa5⁶, Orn¹², Abu¹⁵, Orn²¹, Nle²⁷, Agm²⁹]hGHRH(1-29) NH₂;
P-20111 [N-Me-Tyr¹, D-Ala², Fpa5⁶,Thr⁸, Orn¹², Abu¹⁵, Orn²¹, Nle²⁷, Asp²⁸, Agm²⁹]hGHRH(1-29)NH₂;
P-20113 [N-Me-Tyr¹, Fpa5⁶, Orn²¹, Abu¹⁵, Orn²¹, Nle²⁷, Asp²⁸, Agm²⁹]hGHRH (1-29) NH₂(SEQ ID NO: 4);
P-20115 [N-Me-Tyr¹, Fpa5⁶, Thr⁸, Orn¹², Abu¹⁵, Orn²¹, Nle²⁷, Asp²⁸, Agm²⁹]hGHRH (1-29)NH₂(SEQ ID NO: 5);
P-20117 [N-Me-Tyr¹, D-Ala², Fpa5⁶, Gln⁸, Orn¹², Abu¹⁵, Orn²¹, Nle²⁷, Agm²⁹]hGHRH (1-29) NH₂;
P-20350 [Dat¹, D-Ala², Gln⁸, Orn¹², Abu¹⁵, Orn²¹, Nle²⁷, Asp²⁸, Agm²⁹]hGHRH(1-29) NH₂;
P-20351 [Ac-N-Me-Tyr¹, D-Ala², Gln⁸, Orn¹², Abu¹⁵, Orn²¹, Nle²⁷, Asp²⁸, Agm²⁹]hGHRH(1-29)NH₂;
P-20356 [N-Me-Tyr¹, Gln⁸, Orn¹², Abu¹⁵, Orn²¹, Nle²⁷, Asp²⁸, Agm²⁹]hGHRH(1-29) NH₂ (SEQ ID NO: 6);
P-20357 [Dat¹, D-Ala², N-Me-Ala⁸, Orn¹², Abu¹⁵, Orn²¹, Nle²⁷, Asp²⁸, Agm²⁹]hGHRH (1-29)NH₂;
P-20358 [N-Me-Tyr¹, D-Ala², N-Me-Ala⁸, Orn¹², Abu¹⁵, Orn²¹, Nle²⁷, Asp²⁸, Agm²⁹]hGHRH(1-29)NH₂;
P-20359 [N-Me-Tyr¹, D-Ala², Fpa5⁶, Orn¹², Abu¹⁵, Orn²¹, Nle²⁷, Asp²⁸, Agm²⁹]hGHRH (1-29)NH₂;
P-20360 [N-Me-Tyr¹, D-Ala², Thr⁸, Orn¹², Abu¹⁵, Orn²¹, Nle²⁷, Asp²⁸, Agm²⁹]hGHRH (1-29)NH₂;
P-20361 [N-Me-Tyr¹, D-Ala², Gln⁸, Orn¹², Abu¹⁵, Orn²¹, Nle²⁷, Asp²⁸, Agm²⁹]hGHRH (1-29)NH₂;
P-20367 [N-Me-Tyr¹, D-Ala², Orn¹², Abu¹⁵, Orn²¹, Nle²⁷, Asp²⁸, Agm²⁹]hGHRH (1-29)NH₂;
P-20370 [N-Me-Tyr¹, Orn¹², Abu¹⁵, Orn²¹, Nle²⁷, Asp²⁸, Agm²⁹]hGHRH(1-29)NH₂ (SEQ ID NO: 7);
P-20371 [N-Me-Tyr¹, Thr⁸, Orn¹², Abu¹⁵, Orn²¹, Nle²⁷, Asp²⁸, Agm²⁹]hGHRH (1-29) NH₂ (SEQ ID NO: 8);
P-20372 [N-Me-Tyr¹, Ala⁸, Orn¹², Abu¹⁵, Orn²¹, Nle²⁷, Asp²⁸, Agm²⁹]hGHRH (1-29) NH₂ (SEQ ID NO: 9);
P-20373 [N-Me-Tyr¹, Gln⁸, Orn¹², Abu¹⁵, Orn²¹, Nle²⁷, Agm²⁹]hGHRH(1-29)NH₂ (SEQ ID NO: 10);
P-20374 [N-Me-Tyr¹, Orn¹², Abu¹⁵, Orn²¹, Nle²⁷, Agm²⁹]hGHRH(1-29) NH₂ (SEQ ID NO: 11);
P-20375 [N-Me-Tyr¹, Thr⁸, Orn¹², Abu¹⁵, Orn²¹, Nle²⁷, Agm²⁹]hGHRH(1-29)NH₂ (SEQ ID NO: 12);
P-20376 [N-Me-Tyr¹, Ala⁸, Orn¹², Abu¹⁵, Orn²¹, Nle²⁷, Agm²⁹]hGHRH(1-29)NH₂ (SEQ ID NO: 13);
P-21300 [Dat¹, D-Ala², Orn¹², Abu¹⁵, Orn²¹, Nle²⁷, Asp²⁸, Amc³⁰]hGHRH(1-30)NH₂;
P-21301 [N-Me-Tyr¹, Orn¹², Abu¹⁵, Orn²¹, Nle²⁷, Asp²⁸, Amc³⁰]hGHRH(1-30)NH₂ (SEQ ID NO: 14);
P-21303 [N-Me-Tyr¹, D-Ala², Orn¹², Abu¹⁵, Orn²¹, Nle²⁷, Asp²⁸, Amc³⁰]hGHRH(1-30)NH₂;
P-21304 [Dat¹, D-Ala², Gln⁸, Orn¹², Abu¹⁵, Orn²¹, Nle²⁷, Asp²⁸, Amc³⁰]hGHRH(1-30)NH₂;
P-21305 [N-Me-Tyr¹, D-Ala², Gln⁸, Orn¹², Abu¹⁵, Orn²¹, Nle²⁷, Asp²⁸, Amc³⁰]hGHRH(1-30)NH₂;
P-21306 [Dat¹, D-Ala², Thr⁸, Orn¹², Abu¹⁵, Orn²¹, Nle²⁷, Asp²⁸, Amc³⁰]hGHRH(1-30)NH₂;
P-21307 [N-Me-Tyr¹, D-Ala², Thr⁸, Orn¹², Abu¹⁵, Orn²¹, Nle²⁷, Asp²⁸, Amc³⁰]hGHRH(1-30)NH₂;
P-21308 [Dat¹, D-Ala², Ala⁸, Orn¹², Abu¹⁵, Orn²¹, Nle²⁷, Asp²⁸, Amc³⁰]hGHRH(1-30)NH₂;
P-21309 [N-Me-Tyr¹, D-Ala², Orn¹², Ala⁸, Abu¹⁵, Orn²¹, Nle²⁷, Asp²⁸, Amc³⁰]hGHRH(1-30)NH₂;
P-21310 [Dat¹-D-Ala², His¹¹, Orn¹², Abu¹⁵, His²⁰, Orn²¹, Nle²⁷, Asp²⁸, Amc³⁰]hGHRH(1-30)NH₂;
P-21311 [N-Me-Tyr¹, D-Ala², His¹¹, Orn¹², Abu¹⁵, His²⁰, Orn²¹, Nle²⁷, Asp²⁸, Amc³⁰]hGHRH(1-30)NH₂;
P-22325 [N-Me-Tyr¹, Gln⁸, Orn¹², Abu¹⁵, Orn²¹, Nle²⁷, Asp²⁸, Apa³⁰]hGHRH (1-30)NH₂ (SEQ ID NO: 15);
P-22326 [N-Me-Tyr¹, D-Ala², Gln⁸, Orn¹², Abu¹⁵, Orn²¹, Nle²⁷, Asp²⁸, Apa³⁰]hGHRH(1-30)NH₂;
P-22327 [Dat¹, Gln⁸, Orn¹², Abu¹⁵, Orn²¹, Nle²⁷, Asp²⁸, Apa³⁰]hGHRH(1-30)NH₂ (SEQ ID NO: 16);
P-22328 [Ac-Tyr¹, D-Ala², Gln⁸, Orn¹², Abu¹⁵, Orn²¹, Nle²⁷, Asp²⁸, Apa³⁰]hGHRH(1-30)NH₂;
P-22329 [N-Me-Tyr¹, D-Ala², Orn¹², Abu¹⁵, Orn²¹, Nle²⁷, Apa³⁰]hGHRH(1-30)NH₂;
P-22330 [N-Me-Tyr¹, D-Ala², Thr⁸, Orn¹², Abu¹⁵, Orn²¹, Nle²⁷, Asp²⁸, Apa³⁰]hGHRH(1-30)NH₂;
P-22331 [N-Me-Tyr¹, D-Ala², Ala⁸, Orn¹², Abu¹⁵, Orn²¹, Nle²⁷, Asp²⁸, Apa³⁰]hGHRH(1-30)NH₂;
P-22332 [N-Me-Tyr¹, Orn¹², Abu¹⁵, Orn²¹, Nle²⁷, Asp²⁸, Apa³⁰]hGHRH(1-30)NH₂ (SEQ ID NO: 17);
P-22334 [N-Me-Tyr¹, Orn¹², Abu¹⁵, Orn²¹, Nle²⁷, Apa³⁰]hGHRH(1-30)NH₂ (SEQ ID NO: 18);
P-22335 [N-Me-Tyr¹, Thr⁸, Orn¹², Abu¹⁵, Orn²¹, Nle²⁷, Asp²⁸, Apa³⁰]hGHRH(1-30)NH₂ (SEQ ID NO: 19);
P-22336 [N-Me-Tyr¹ Ala⁸, Orn¹², Abu¹⁵, Orn²¹, Nle²⁷, Asp²⁸, Apa³⁰]hGHRH(1-30)NH₂ (SEQ ID NO: 20);
P-22337 [N-Me-Tyr¹, D-Ala², Cpa⁶, Gln⁸, Orn¹², Abu¹⁵, Orn²¹, Nle²⁷, Asp²⁸, Apa³⁰]hGHRH(1-30)NH₂;
P-23250 [Dat¹, D-Ala², Gln⁸, Orn¹², Abu¹⁵, Orn²¹, Nle²⁷, Asp²⁸, Har³⁰]hGHRH(1-30)NH₂;
P-23251 [Dat¹, Gln⁸, Orn¹², Abu¹⁵, Orn²¹, Nle²⁷, Asp²⁸, Har³⁰]hGHRH(1-30)NH₂ (SEQ ID NO: 21);
P-23252 [Dat¹-D-Ala², Orn¹², Abu¹⁵, Orn²¹, Nle²⁷, Asp²⁸, Har³⁰]hGHRH(1-30)NH₂;
P-23253 [Dat¹, Orn¹², Abu¹⁵, Orn²¹, Nle²⁷, Asp²⁸, Har³⁰]hGHRH(1-30)NH₂ (SEQ ID NO: 22);
P-23254 [Dat¹, D-Ala², Thr⁸, Orn¹², Abu¹⁵, Orn²¹, Nle²⁷, Asp²⁸, Har³⁰]hGHRH(1-30)NH₂;
P-23255 [Dat¹, Thr⁸, Orn¹², Abu¹⁵, Orn²¹, Nle²⁷, Asp²⁸, Har³⁰]hGHRH(1-30)NH₂ (SEQ ID NO: 23);
P-23256 [Dat¹, D-Ala², Ala⁸, Orn¹², Abu¹⁵, Orn²¹, Nle²⁷, Asp²⁸, Har³⁰]hGHRH(1-30)NH₂;
P-23257 [Dat¹, Ala⁸, Orn¹², Abu¹⁵, Orn²¹, Nle²⁷, Asp²⁸, Har³⁰]hGHRH(1-30)NH₂ (SEQ ID NO: 24);
P-23258 [N-Me-Tyr¹, D-Ala², Gln⁸, Orn¹², Abu¹⁵, Orn²¹, Nle²⁷, Asp²⁸, Har³⁰]hGHRH(1-30)NH₂;
P-23259 [N-Me-Tyr¹, Gln⁸, Orn¹², Abu¹⁵, Orn²¹, Nle²⁷, Asp²⁸, Har³⁰]hGHRH(1-30)NH₂(SEQ ID NO: 25);
P-23260 [N-Me-Tyr¹, D-Ala², Orn¹², Abu¹⁵, Orn²¹, Nle²⁷, Asp²⁸, Har³⁰]hGHRH(1-30)NH₂;
P-23261 [N-Me-Tyr¹, Orn¹², Abu¹⁵, Orn²¹, Nle²⁷, Asp²⁸, Har³⁰]hGHRH(1-30)NH₂ (SEQ ID NO: 26);
P-23262 [N-Me-Tyr¹, D-Ala², Thr⁸, Orn¹², Abu¹⁵, Orn²¹, Nle²⁷, Asp²⁸, Har³⁰]hGHRH(1-30)NH₂;
P-23263 [N-Me-Tyr¹, Thr⁸, Orn¹², Abu¹⁵, Orn²¹, Nle²⁷, Asp²⁸, Har³⁰]hGHRH(1-30)NH₂ (SEQ ID NO: 27);
P-23264 [N-Me-Tyr¹, D-Ala², Ala⁸, Orn¹², Abu¹⁵, Orn²¹, Nle²⁷, Asp²⁸, Har³⁰]hGHRH(1-30)NH₂;
P-23265 [N-Me-Tyr¹, Ala⁸, Orn¹², Abu¹⁵, Orn²¹, Nle²⁷, Asp²⁸, Har³⁰]hGHRH(1-30)NH₂ (SEQ ID NO: 28);
P-24340 [N-Me-Tyr¹, Gln⁸, Orn¹², Abu¹⁵, Orn²¹, Nle²⁷, Asp²⁸, Aha³⁰]hGHRH(1-30)NH₂ (SEQ ID NO: 29);
P-24341 [N-Me-Tyr¹, D-Ala², Gln⁸, Orn¹², Abu¹⁵, Orn²¹, Nle²⁷, Asp²⁸, Aha³⁰]hGHRH(1-30)NH₂;
P-24342 [Dat¹, Gln⁸, Orn¹², Abu¹⁵, Orn²¹, Nle²⁷, Asp²⁸, Aha³⁰]hGHRH(1-30)NH₂ (SEQ ID NO: 30);
P-24344 [Dat¹-D-Ala², Gln⁸, Orn¹², Abu¹⁵, Orn²¹, Nle²⁷, Asp²⁸, Aha³⁰]hGHRH(1-30)NH₂;
P-24345 [N-Me-Tyr¹, D-Ala², Orn¹², Abu¹⁵, Orn²¹, Nle²⁷, Asp²⁸, Aha³⁰]hGHRH(1-30)NH₂;
P-24346 [N-Me-Tyr¹, D-Ala², Gln⁸, Orn¹², Abu¹⁵, Orn²¹, Nle²⁷, Aha³⁰]hGHRH (1-30)NH₂;
P-24347 [N-Me-Tyr¹, D-Ala², Thr⁸, Orn¹², Abu¹⁵, Orn²¹, Nle²⁷, Asp²⁸, Aha³⁰]hGHRH(1-30)NH₂;
P-24348 [N-Me-Tyr¹, D-Ala², Ala⁸, Orn¹², Abu¹⁵, Orn²¹, Nle²⁷, Asp²⁸, Aha³⁰]hGHRH(1-30)NH₂;
P-25501 [N-Me-Tyr¹, D-Ala², Orn¹², Abu¹⁵, Orn²¹, Nle²⁷, Gab³⁰]hGHRH(1-30)NH₂;
P-25502 [Dat¹, D-Ala², Fpa5⁶, Orn¹², Abu¹⁵, Orn²¹, Nle²⁷, Gab³⁰]hGHRH(1-30)NH₂;
P-25503 [N-Me-Tyr¹, Abu², Orn¹², Abu¹⁵, Orn²¹, Nle²⁷, Gab³⁰]hGHRH(1-30)NH₂ (SEQ ID NO: 31);
P-25504 [Dat¹, D-Abu², Orn¹², Abu¹⁵, Orn²¹, Nle²⁷, Gab³⁰]hGHRH(1-30)NH₂;
P-25506 [N-Me-Tyr¹, D-Abu², Orn¹², Abu¹⁵, Orn²¹, Nle²⁷, Gab³⁰]hGHRH(1-30) NH₂;
P-25508 [Tfa-Tyr¹, D-Ala², Orn¹², Abu¹⁵, Orn²¹, Nle²⁷, Gab³⁰]hGHRH(1-30)NH₂;
P-25516 [N-Me-Tyr¹, D-Ala², Orn¹², Abu¹⁵, Orn²¹, Nle²⁷, Asp²⁸, Gab³⁰]hGHRH(1-30)NH₂;
P-26802 [Dat¹, D-Ala², Thr⁸, His¹¹, Orn¹², Abu¹⁵, His²⁰, Orn²¹, Nle²⁷, Ada³⁰]hGHRH(1-30)NH₂;
P-26803 [N-Me-Tyr¹, D-Ala², Gln⁸, His¹¹, Orn¹², Abu¹⁵, His²⁰, Orn²¹, Nle²⁷, Ada³⁰]hGHRH(1-30)NH—CH₃;
P-26804 [N-Me-Tyr¹, D-Ala², Gln⁸, Orn¹², Abu¹⁵, Orn²¹, Nle²⁷, Ada³⁰]hGHRH(1-30)NH₂;
P-27400 [Dat¹, D-Ala², Orn¹², Abu¹⁵, Orn²¹, Nle²⁷, Asp²⁸]hGHRH(1-29)NH—CH₃;
P-27401 [Dat¹, D-Ala², Gln⁸, Orn¹², Abu¹⁵, Orn²¹, Nle²⁷, Asp²⁸, D-Arg²⁹]hGHRH(1-29)NH—CH₃;
P-27403 [N-Me-Tyr¹, D-Ala², Gln⁸, Orn¹², Abu¹⁵, Orn²¹, Nle²⁷, Asp²⁸]hGHRH(1-29)NH—CH₃;
P-27404 [N-Me-Tyr¹, D-Ala², Fpa5⁶, Orn¹², Abu¹⁵, Orn²¹, Nle²⁷, Asp²⁸]hGHRH (1-29)NH—CH₃;
P-27405 [N-Me-Tyr¹, Gln⁸, Orn¹², Abu¹⁵, Orn²¹, Nle²⁷]hGHRH(1-29)NH—CH₃ (SEQ ID NO: 32);
P-27406 [N-Me-Tyr¹, Gln⁸, Orn¹², Abu¹⁵, Orn²¹, Nle²⁷, Asp²⁸]hGHRH(1-29) NH—CH₃ (SEQ ID NO: 33);
P-27407 [Dat¹, Gln⁸, Orn¹², Abu¹⁵, Orn²¹, Nle²⁷, Asp²⁸]hGHRH(1-29)NH—CH₃ (SEQ ID NO: 34);
P-27408 [Dat¹, D-Ala², Gln⁸, Orn¹², Abu¹⁵, Orn²¹, Nle²⁷, Asp²⁸]hGHRH(1-30)NH—CH₃;
P-27409 [N-Me-Tyr¹, D-Ala², Orn¹², Abu¹⁵, Orn²¹, Nle²⁷, Asp²⁸]hGHRH(1-29)NH—CH₃;
P-27410 [N-Me-Tyr¹, D-Ala², Thr⁸, Orn¹², Abu¹⁵, Orn²¹, Nle²⁷, Asp²⁸]hGH RH(1-29)NH—CH₃;
P-27411 [N-Me-Tyr¹, D-Ala², Thr⁸, Orn¹², Abu¹⁵, Orn²¹, Nle²⁷]hGHRH(1-29) NH—CH₃;
P-27412 [N-Me-Tyr¹, D-Ala², Ala⁸, Orn¹², Abu¹⁵, Orn²¹, Nle²⁷, Asp²⁸]hGHRH (1-29)NH—CH₃;
P-27413 [Dat¹, Gln⁸, His¹¹, Orn¹², Abu¹⁵, His²⁰, Orn²¹, Nle²⁷, Asp²⁸]hGHRH (1-29)NH—CH₃(SEQ ID NO: 35);
P-27414 [N-Me-Tyr¹, D-Ala², Fpa5⁶, Orn¹², Abu¹⁵, Orn²¹, Nle²⁷, Gab³⁰]hGHRH(1-30)NH—CH₃;
P-27415 [N-Me-Tyr¹, Orn¹², Abu¹⁵, Orn²¹, Nle²⁷, Gab³⁰]hGHRH(1-30)NH—CH₃ (SEQ ID NO: 36);
P-27416 [N-Me-Tyr¹, D-Ala², Gln⁸, His¹¹, Orn¹², Abu¹⁵, His²⁰, Orn²¹, Nle²⁷, Asp²⁸]hGHRH(1-29)NH—CH₃;
P-27417 [Ac-Tyr¹, D-Ala², Gln⁸, Orn¹², Abu¹⁵, Orn²¹, Nle²⁷, Asp²⁸]hGHRH(1-29) NH—CH₃;
P-27418 [Ac-Tyr¹, D-Ala², Fpa5⁶, Orn¹², Abu¹⁵, Orn²¹, Nle²⁷, Asp²⁸]hGHRH(1-29)NH—CH₃;
P-27419 [Ac-Tyr¹, Thr⁸, Orn¹², Abu¹⁵, Orn²¹, Nle²⁷]hGHRH(1-29)NH—CH₃ (SEQ ID NO: 37);
P-27422 [N-Me-D-Tyr¹, Gln⁸, Orn¹², Abu¹⁵, Orn²¹, Nle²⁷, Asp²⁸]hGHRH(1-29) NH—CH₃;
P-27423 [N-Me-D-Tyr¹, D-Ala², Orn¹², Abu¹⁵, Orn²¹, Nle²⁷, Asp²]hGHRH(1-29)NH—CH₃;
P-27424 [Dat¹, Thr⁸, Orn¹², Abu¹⁵, Orn²¹, Nle²⁷]hGHRH(1-29)NH—CH₃ (SEQ ID NO: 38);
P-27425 [N-Me-D-Tyr¹, D-Ala², Gln⁸, Orn¹², Abu¹⁵, Orn²¹, Nle²⁷, Asp²⁸]hGHRH(1-29)NH—CH₃;
P-27440 [Dat¹, Gln⁸, Orn¹², Abu¹⁵, Orn²¹, Nle²⁷, Asp²⁸, D-Arg²⁹]hGHRH(1-29)NH—CH₃;
P-27441 [N-Me-Tyr¹, D-Ala², Gln⁸, Orn¹², Abu¹⁵, Orn²¹, Nle²⁷, Asp²⁸, D-Arg²⁹]hGHRH(1-29)NH—CH₃;
P-27442 [N-Me-Tyr¹, D-Ala², Fpa5⁶, Orn¹², Abu¹⁵, Orn²¹, Nle²⁷, Asp²⁸, D-Arg²⁹]hGHRH(1-29)NH—CH₃;
P-27443 [N-Me-Tyr¹, Orn¹², Abu¹⁵, Orn²¹, Nle²⁷, D-Arg²⁹]hGHRH(1-29)NH—CH₃;
P-27444 [N-Me-Tyr¹, D-Ala², Orn¹², Abu¹⁵, Orn²¹, Nle²⁷, Asp²⁸, D-Arg²⁹]hGHRH(1-29)NH—CH₃;
P-27445 [N-Me-Tyr¹, D-Ala², Thr⁸, Orn¹², Abu¹⁵, Orn²¹, Nle²⁷, Asp²⁸, D-Arg²⁹]hGHRH(1-29)NH—CH₃;
P-27446 [N-Me-Tyr¹, D-Ala², Ala⁸, Orn¹², Abu¹⁵, Orn²¹, Nle²⁷, Asp²⁸, D-Arg²⁹]hGHRH(1-29)NH—CH₃;
P-27447 [N-Me-Tyr¹, D-Ala², Gln⁸, Orn¹², Abu¹⁵, Orn²¹, Nle²⁷, Asp²⁸, Apa³⁰]hGHRH(1-30)NH—CH₃;
P-27448 [N-Me-Tyr¹, D-Ala², Gln⁸, Orn¹², Abu¹⁵, Orn²¹, Nle²⁷, Asp²⁸, Aha³⁰]hGHRH(1-30)NH—CH₃;
P-27449 [N-Me-Tyr¹, D-Ala², Gln⁸, Orn¹², Abu¹⁵, Orn²¹, Nle²⁷, Asp²⁸, Amc³⁰]hGHRH(1-30)NH—CH₃;
P-27450 [N-Me-Tyr¹, D-Ala², Gln⁸, Orn¹², Abu¹⁵, Orn²¹, Nle²⁷, Asp²⁸, Har³⁰]hGHRH(1-30)NH—CH₃;
P-27451 [N-Me-Tyr¹, D-Ala², Gln⁸, His¹¹, Orn¹², Abu¹⁵, His²⁰, Orn²¹, Nle²⁷, Asp²⁸, Apa³⁰]hGHRH(1-30)NH—CH₃;
P-28420 [N-Me-Tyr¹, D-Ala², Gln⁸, Orn¹², Abu¹⁵, Orn²¹, Nle²⁷, Asp²⁸]hGHRH(1-29)NH—CH₂—CH₃;
P-28421 [N-Me-Tyr¹, D-Ala², Orn¹², Abu¹⁵, Orn²¹, Nle²⁷, Asp²⁸]hGHRH(1-29) NH—CH₂—CH₃;
P-28430 [N-Me-Tyr¹, D-Ala², Gln⁸, Orn¹², Abu¹⁵, Orn²¹, Nle²⁷]hGHRH(1-29) NH—CH₂—CH₃;
P-28431 [N-Me-Tyr¹, D-Ala², Thr⁸, Orn¹², Abu¹⁵, Orn²¹, Nle²⁷, Asp²⁸]hGHRH(1-29)NH—CH₂—CH₃;
P-28460 [N-Me-Tyr¹, D-Ala², Fpa5⁶, Gln⁸, Orn¹², Abu¹⁵, Orn²¹, Nle²⁷, Asp²⁸]hGHRH(1-29)NH—CH₂—CH₃;
P-28461 [N-Me-Tyr¹, D-Ala², Gln⁸, Orn¹², Abu¹⁵, Orn²¹, Nle²⁷]hGHRH(1-29) NH—CH₂—CH₃;
P-28462 [N-Me-Tyr¹, D-Ala², Fpa5⁶, Orn¹², Abu¹⁵, Orn²¹, Nle²⁷]hGHRH(1-29) NH—CH₂—CH₃;
P-28463 [N-Me-Tyr¹, D-Ala², Orn¹², Abu¹⁵, Orn²¹, Nle²⁷]hGHRH(1-29)NH—CH₂—CH₃;
P-28464 [N-Me-Tyr¹, D-Ala², Fpa5⁶, Gln⁸, Orn¹², Abu¹⁵, Orn²¹, Nle²⁷]hGHRH (1-29)NH—CH₂—CH₃;
P-28465 [N-Me-Tyr¹, D-Ala², Gln⁸, Orn¹², Abu¹⁵, Orn²¹, Nle²⁷, Asp²⁸]hGHRH(1-29)NH—CH₂—CH₃;
P-28466 [N-Me-Tyr¹, D-Ala², Fpa5⁶, Orn¹², Abu¹⁵, Orn²¹, Nle²⁷, Asp²⁸]hGHRH (1-29)NH—CH₂—CH₃;
P-28467 [N-Me-Tyr¹, D-Ala², Orn¹², Abu¹⁵, Orn²¹, Nle²⁷, Asp²⁸]hGHRH(1-29) NH—CH₂—CH₃;
P-28468 [N-Me-Tyr¹, D-Ala², Fpa5⁶, Gln⁸, Orn¹², Abu¹⁵, Orn²¹, Nle²⁷, Asp²⁸]hGHRH(1-29)NH—CH₂—CH₃;
P-28469 [Dat¹, D-Ala², Gln⁸, Orn¹², Abu¹⁵, Orn²¹, Nle²⁷]hGHRH(1-29)NH—CH₂—CH₃;
P-28470 [Dat¹, D-Ala², Orn¹², Abu¹⁵, Orn²¹, Nle²⁷]hGHRH(1-29)NH—CH₂—CH₃;
P-28471 [Dat¹, D-Ala², Gln⁸, Orn¹², Abu¹⁵, Orn²¹, Nle²⁷, Asp²⁸]hGHRH(1-29)NH—CH₂—CH₃;
P-28472 [Dat¹, D-Ala², Fpa5⁶, Gln⁸, Orn¹², Abu¹⁵, Orn²¹, Nle²⁷, Asp²⁸]hGHRH(1-29)NH—CH₂—CH₃;
P-28473 [Dat¹, D-Ala², Fpa5⁶, Orn¹², Abu¹⁵, Orn²¹, Nle²⁷, Asp²⁸]hGHRH(1-29)NH—CH₂—CH₃;
P-28474 [Dat¹, D-Ala², Orn¹², Abu¹⁵, Orn²¹, Nle²⁷, Asp²⁸]hGHRH(1-29)NH—CH₂—CH₃;
P-28475 [N-Me-Tyr¹, D-Ala², Gln⁸, Orn¹², Abu¹⁵, Orn²¹, Nle²⁷, Asp²⁸, Apa³⁰]hGHRH(1-30)NH—CH₂—CH₃;
P-28476 [N-Me-Tyr¹, D-Ala², Gln⁸, Orn¹², Abu¹⁵, Orn²¹, Nle²⁷, Asp²⁸, Aha³⁰]hGHRH(1-30)NH—CH₂—CH₃;
P-28477 [N-Me-Tyr¹, D-Ala², Gln⁸, Orn¹², Abu¹⁵, Orn²¹, Nle²⁷, Asp²⁸, Amc³⁰]hGHRH(1-30)NH—CH₂—CH₃;
P-28478 [N-Me-Tyr¹, D-Ala², Gln⁸, Orn¹², Abu¹⁵, Orn²¹, Nle²⁷, Asp²⁸, Har³⁰]hGHRH(1-30)NH—CH₂—CH₃;
P-28479 [N-Me-Tyr¹, D-Ala², Gln⁸, His¹¹, Orn¹², Abu¹⁵, His²⁰, Orn²¹, Nle²⁷, Asp²⁸, Apa³⁰]hGHRH(1-30)NH—CH₂—CH₃;
P-29701 [N-Me-Tyr¹, D-Ala², Gln⁸, Orn¹², Abu¹⁵, Orn²¹, Nle²⁷, Asp²⁸, Gln-Gab³⁰]hGHRH](1-30)NH₂;
P-29702 [Dat¹, D-Ala², Orn¹², Abu¹¹, Orn²¹, Nle²⁷, Gln-Gab³⁰]hGHRH(1-30)NH₂;
P-29703 [N-Me-Tyr¹,Gln⁸, Orn¹², Abu¹⁵, Orn²¹, Nle²⁷, Gln-Gab³⁰]hGHRH(1-30)NH₂ (SEQ ID NO: 39);
P-29704 [Dat¹, D-Ala², Gln⁸, Orn¹², Abu¹⁵, Orn^{21,22}, Nle²⁷, Gln-Gab³⁰]hGHRH(1-30)NH₂;
P-29706 [Tfa-Tyr¹, D-Abu², Gln⁸, Orn¹², Abu¹⁵, Orn²¹, Nle²⁷, Gln-Gab³⁰]hGHRH(1-30)NH₂;
P-29708 [N-Me-Tyr¹, D-Ala², Fpa5⁶, Orn¹², Abu¹⁵, Orn²¹, Nle²⁷, Gln-Gab³⁰]hGHRH(1-30)NH₂;
P-29710 [N-Me-Tyr¹, D-Ala², Fpa5⁶, Ala⁸, Orn¹², Abu¹⁵, Orn²¹, Nle²⁷, Gln-Gab³⁰]hGHRH(1-30)NH₂;
P-29720 [Dat¹, D-Ala², Orn¹², Abu¹⁵, Orn²¹, Nle²⁷, Asp²⁸, Gln-Gab³⁰]hGHRH(1-30)NH₂;
P-29721 [Dat¹, D-Ala², Gln⁸, Orn¹², Abu¹⁵, Orn²¹²², Nle²⁷, Asp²⁸, Gln-Gab³⁰]hGHRH(1-30) NH₂;
P-29722 [Tfa-Tyr¹, D-Abu², Gln⁸, Orn¹², Abu¹⁵, Orn²¹, Nle²⁷, Asp²⁸, Gln³⁰, Gab³¹]hGHRH(1-30)NH₂;
P-29723 [N-Me-Tyr¹, D-Ala², Fpa5⁶, Orn¹², Abu¹⁵, Orn²¹, Nle²⁷, Asp²⁸, Gln-Gab³⁰]hGHRH (1-30)NH₂;
P-29724 [N-Me-Tyr¹, D-Ala², Fpa5⁶, Ala⁸, Orn¹², Abu¹⁵, Orn²¹, Nle²⁷, Asp²⁸, Gln Gab³⁰]hGHRH(1-30)NH₂;
JI-34 [Dat¹, Orn¹², Abu¹⁵, Orn²¹, Nle²⁷, Asp²⁸, Agm²⁹]hGHRH(1-29)NH₂ (SEQ ID NO: 40);
JI-36 [Dat¹, Thr⁸, Orn¹², Abu¹⁵, Orn²¹, Nle²⁷, Asp²⁸, Agm²⁹]hGHRH(1-29)NH₂ (SEQ ID NO: 41); and
JI-38 [Dat¹, Gln⁸, Orn¹², Abu¹⁵, Orn²¹, Nle²⁷, Asp²⁸, Agm²⁹]hGHRH(1-29)NH₂ (SEQ ID NO: 42).

12. The method of claim 1, wherein the GHRH agonist is selected from:
P-20350 [Dat¹, D-Ala², Gln⁸, Orn¹², Abu¹⁵, Orn²¹, Nle²⁷, Asp²⁸, Agm²⁹]hGHRH(1-29) NH₂;
P-20357 [Dat¹, D-Ala², N-Me-Ala⁸, Orn¹², Abu¹⁵, Orn²¹, Nle²⁷, Asp²⁸, Agm²⁹]hGHRH (1-29)NH₂;
P-21300 [Dat¹, D-Ala², Orn¹², Abu¹⁵, Orn²¹, Nle²⁷, Asp²⁸, Amc³⁰]hGHRH(1-30)NH₂;
P-21304 [Dat¹, D-Ala², Gln⁸, Orn¹², Abu¹⁵, Orn²¹, Nle²⁷, Asp²⁸, Amc³⁰]hGHRH(1-30)NH₂;
P-21306 [Dat¹, D-Ala², Thr⁸, Orn¹², Abu¹⁵, Orn²¹, Nle²⁷, Asp²⁸, Amc³⁰]hGHRH(1-30)NH₂;
P-21308 [Dat¹, D-Ala², Ala⁸, Orn¹², Abu¹⁵, Orn²¹, Nle²⁷, Asp²⁸, Amc³⁰]hGHRH(1-30)NH₂;
P-21310 [Dat¹-D-Ala², His¹¹, Orn¹², Abu¹⁵, His²⁰, Orn²¹, Nle²⁷, Asp²⁸, Amc³⁰]hGHRH(1-30)NH₂;
P-22327 [Dat¹, Gln⁸, Orn¹², Abu¹⁵, Orn²¹, Nle²⁷, Asp²⁸, Apa³⁰]hGHRH(1-30)NH₂ (SEQ ID NO: 16);
P-23250 [Dat¹, D-Ala², Gln⁸, Orn¹², Abu¹⁵, Orn²¹, Nle²⁷, Asp²⁸, Har³⁰]hGHRH(1-30)NH₂;
P-23251 [Dat¹, Gln⁸, Orn¹², Abu¹⁵, Orn²¹, Nle²⁷, Asp²⁸, Har³⁰]hGHRH(1-30)NH₂ (SEQ ID NO: 21);
P-23252 [Dat¹-D-Ala², Orn¹², Abu¹⁵, Orn²¹, Nle²⁷, Asp²⁸, Har³⁰]hGHRH(1-30)NH₂;
P-23253 [Dat¹, Orn¹², Abu¹⁵, Orn²¹, Nle²⁷, Asp²⁸, Har³⁰]hGHRH(1-30)NH₂ (SEQ ID NO: 22);
P-23254 [Dat¹, D-Ala², Thr⁸, Orn¹², Abu¹⁵, Orn²¹, Nle²⁷, Asp²⁸, Har³⁰]hGHRH(1-30)NH₂;
P-23255 [Dat¹, Thr⁸, Orn¹², Abu¹⁵, Orn²¹, Nle²⁷, Asp²⁸, Har³⁰]hGHRH(1-30)NH₂ (SEQ ID NO: 23);
P-23256 [Dat¹, D-Ala², Ala⁸, Orn¹², Abu¹⁵, Orn²¹, Nle²⁷, Asp²⁸, Har³⁰]hGHRH(1-30)NH₂;
P-23257 [Dat¹, Ala⁸, Orn¹², Abu¹⁵, Orn²¹, Nle²⁷, Asp²⁸, Har³⁰]hGHRH(1-30)NH₂ (SEQ ID NO: 24);
P-24342 [Dat¹, Gln⁸, Orn¹², Abu¹⁵, Orn²¹, Nle²⁷, Asp²⁸, Aha³⁰]hGHRH(1-30)NH₂ (SEQ ID NO: 30);
P-24344 [Dat¹-D-Ala², Gln⁸, Orn¹², Abu¹⁵, Orn²¹, Nle²⁷, Asp²⁸, Aha³⁰]hGHRH(1-30)NH₂;
P-25502 [Dat¹, D-Ala², Fpa5⁶, Orn¹², Abu¹⁵, Orn²¹, Nle²⁷, Gab³⁰]hGHRH(1-30)NH₂;
P-25504 [Dat¹, D-Abu², Orn¹², Abu¹⁵, Orn²¹, Nle²⁷, Gab³⁰]hGHRH(1-30)NH₂;
P-26802 [Dat¹, D-Ala², Thr⁸, His¹¹, Orn¹², Abu¹⁵, His²⁰, Orn²¹, Nle²⁷, Ada³⁰]hGHRH(1-30)NH₂;
P-27400 [Dat¹, D-Ala², Orn¹², Abu¹⁵, Orn²¹, Nle²⁷, Asp²⁸]hGHRH(1-29)NH—CH₃;
P-27401 [Dat¹, D-Ala², Gln⁸, Orn¹², Abu¹⁵, Orn²¹, Nle²⁷, Asp²⁸, D-Arg²⁹]hGHRH(1-29)NH—CH₃;
P-27407 [Dat¹, Gln⁸, Orn¹², Abu¹⁵, Orn²¹, Nle²⁷, Asp²⁸]hGHRH(1-29)NH—CH₃ (SEQ ID NO: 34);
P-27408 [Dat¹, D-Ala², Gln⁸, Orn¹², Abu¹⁵, Orn²¹, Nle²⁷, Asp²⁸]hGHRH(1-30)NH—CH₃;
P-27413 [Dat¹, Gln⁸, His¹¹, Orn¹², Abu¹⁵, His²⁰, Orn²¹, Nle²⁷, Asp²⁸]hGHRH (1-29)NH—CH₃ (SEQ ID NO: 35);
P-27424 [Dat¹, Thr⁸, Orn¹², Abu¹⁵, Orn²¹, Nle²⁷]hGHRH(1-29)NH—CH₃ (SEQ ID NO: 38);
P-27440 [Dat¹, Gln⁸, Orn¹², Abu¹⁵, Orn²¹, Nle²⁷, Asp²⁸, D-Arg²⁹]hGHRH(1-29)NH—CH₃;
P-28469 [Dat¹, D-Ala², Gln⁸, Orn¹², Abu¹⁵, Orn²¹, Nle²⁷]hGHRH(1-29)NH—CH₂—CH₃;
P-28470 [Dat¹, D-Ala², Orn¹², Abu¹⁵, Orn²¹, Nle²⁷]hGHRH(1-29)NH—CH₂—CH₃;
P-28471 [Dat¹, D-Ala², Gln⁸, Orn¹², Abu¹⁵, Orn²¹, Nle²⁷, Asp²⁸]hGHRH(1-29)NH—CH₂—CH₃;
P-28472 [Dat¹, D-Ala², Fpa5⁶, Gln⁸, Orn¹², Abu¹⁵, Orn²¹, Nle²⁷, Asp²⁸]hGHRH(1-29)NH—CH₂—CH₃;
P-28473 [Dat¹, D-Ala², Fpa5⁶, Orn¹², Abu¹⁵, Orn²¹, Nle²⁷, Asp²⁸]hGHRH(1-29)NH—CH₂—CH₃;
P-28474 [Dat¹, D-Ala², Orn¹², Abu¹⁵, Orn²¹, Nle²⁷, Asp²⁸]hGHRH(1-29)NH—CH₂—CH₃;
P-29702 [Dat¹, D-Ala², Orn¹², Abu¹⁵, Orn²¹, Nle²⁷, Gln-Gab³⁰]hGHRH(1-30)NH₂;
P-29704 [Dat¹, D-Ala², Gln⁸, Orn¹², Abu¹⁵, Orn^{21,22}, Nle²⁷, Gln-Gab³⁰]hGHRH(1-30)NH₂;
P-29720 [Dat¹, D-Ala², Orn¹², Abu¹⁵, Orn²¹, Nle²⁷, Asp²⁸, Gln-Gab³⁰]hGHRH(1-30)NH₂;
P-29721 [Dat¹, D-Ala², Gln⁸, Orn¹², Abu¹⁵, Orn²¹²², Nle²⁷, Asp²⁸, Gln-Gab³⁰]hGHRH(1-30) NH₂;
JI-34 [Dat¹, Orn¹², Abu¹⁵, Orn²¹, Nle²⁷, Asp²⁸, Agm²⁹]hGHRH(1-29)NH₂ (SEQ ID NO: 40);
JI-36 [Dat¹, Thr⁸, Orn¹², Abu¹⁵, Orn²¹, Nle²⁷, Asp²⁸, Agm²⁹]hGHRH(1-29)NH₂ (SEQ ID NO: 41); and
JI-38 [Dat¹, Gln⁸, Orn¹², Abu¹⁵, Orn²¹, Nle²⁷, Asp²⁸, Agm²⁹]hGHRH(1-29)NH₂(SEQ ID NO: 42).

13. The method of claim 1, further comprising administering the GHRH agonist peptide to the subject before the cancer treatment, concurrently with the cancer treatment, post-treatment, or during remission of the cancer.

14. The method of claim 1, wherein the at least one anticancer agent is selected from tamoxifen, toremifen, raloxifene, droloxifene, iodoxyfene, megestrol acetate, anasfrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, vincristine, taxol, taxotere, etoposide, teniposide, amsacrine, Irinotecan, topotecan, epothilones, gefitinib, erlotinib, angiogenesis inhibitors, EGF inhibitors, VEGF inhibitors, CDK inhibitors, cytokines, Her1 and Her2 inhibitors, and monoclonal antibodies.

15. The method of claim 1, wherein the at least one GHRH agonist peptide is co-administered, concurrently administered, or sequentially administered with at least one anticancer agent.

16. The method of claim 1, wherein the GHRH agonist peptide is JI-34 and the anticancer agent is doxorubicin.

17. A method of killing cancer cells, the method comprising contacting the cancer cells with a composition comprising at least one GHRH agonist peptide and at least one anticancer agent.

18. The method of claim 17, wherein the at least one GHRH agonist peptide has the formula II:
[R₁-A¹-A²-Asp-Ala-Ile-A⁶-Thr-A⁸-Ser-Tyr-A¹¹-A¹²-Val-Leu-A¹⁵-Gln-Leu-Ser-Ala-A²⁰-A²¹-A²²-Leu-Gln-Asp-Ile-Nle²⁷-A²⁸-A²⁹-A³⁰]-R₂,
wherein
R₁ is Ac, Tfa, or is absent,
A¹ is Tyr, Dat, or N-Me-Tyr,
A² is Ala, D-Ala, Abu, or D-Abu,
A⁶ is Phe or Fpa5,
A⁸ is Asn, Ala, Gln, Thr, or N-Me-Ala,
A¹¹ is Arg, His, or Har,
A¹² is Orn, or Lys(Me)₂,
A¹⁵ is Abu or Ala,
A²⁰ is Arg, His, or Har,
A²¹ is Orn, or Lys(Me)₂,
A²² is Leu, or Orn,
A²⁸ is Ser, or Asp,
A²⁹ is Arg, Har, Agm, D-Arg, or D-Har,
A³⁰ is Arg, Agm, Ada, Amc, Aha, Apa, Har, D-Arg, D-Har, Gab, Gln, D-Gln, Gln-Gab, D-Gln-Gab, or is absent,
R₂ is —NH₂, —NH—CH₃, NH—CH₂—CH₃, —NH—CH₃, —OH, —NHR₃, —N(R₃)₂, or —OR₃, wherein R₃ is any of C₁₋₁₂ alkyl, C₂₋₁₂ alkenyl, or C₂₋₁₂ alkynyl,
and pharmaceutically acceptable salts thereof, and wherein the GHRH agonist peptide is different from SEQ ID NO: 1 in at least one amino acid residue.

19. The method of claim 17, wherein the cancer cell is selected from a colorectal cancer cell, a breast cancer cell, an ovarian cancer cell, a pancreatic cancer cell, a head and neck cancer cell, a bladder cancer cell, a liver cancer cell, a renal cancer cell, a melanoma cell, a gastrointestinal cancer cell, a prostate cancer cell, a small cell lung cancer cell, non-small cell lung cancer cell, a sarcoma cell, a glioblastoma cell, T- and B-cell lymphoma cell, a endometrial cancer cell, and a cervical cancer cell.

20. A method of killing cancer stem cells, the method comprising contacting the cancer stem cells with a composition comprising at least one GHRH agonist peptide and at least one anticancer agent.

21. The method of claim 20, wherein the at least one GHRH agonist peptide has the formula II:
[R₁-A¹-A²-Asp-Ala-Ile-A⁶-Thr-A⁸-Ser-Tyr-A¹¹-A¹²-Val-Leu-A¹⁵-Gln-Leu-Ser-Ala-A²⁰-A²¹-A²²-Leu-Gln-Asp-Ile-Nle²⁷-A²⁸-A²⁹-A³⁰]-R₂,
wherein
R₁ is Ac, Tfa, or is absent,
A¹ is Tyr, Dat, or N-Me-Tyr,
A² is Ala, D-Ala, Abu, or D-Abu,
A⁶ is Phe or Fpa5,
A⁸ is Asn, Ala, Gln, Thr, or N-Me-Ala,
A¹¹ is Arg, His, or Har,
A¹² is Orn, or Lys(Me)₂,
A¹⁵ is Abu or Ala,
A²⁰ is Arg, His, or Har,
A²¹ is Orn, or Lys(Me)₂,
A²² is Leu, or Orn,
A²⁸ is Ser, or Asp,
A²⁹ is Arg, Har, Agm, D-Arg, or D-Har,
A³⁰ is Arg, Agm, Ada, Amc, Aha, Apa, Har, D-Arg, D-Har, Gab, Gln, D-Gln, Gln-Gab, D-Gln-Gab, or is absent,
R₂ is —NH₂, —NH—CH₃, NH—CH₂—CH₃, —NH—CH₃, —OH, —NHR₃, —N(R₃)₂, or —OR₃, wherein R₃ is any of C₁₋₁₂ alkyl, C₂₋₁₂ alkenyl, or C₂₋₁₂ alkynyl,
and pharmaceutically acceptable salts thereof, and wherein the GHRH agonist peptide is different from SEQ ID NO: 1 in at least one amino acid residue.

22. The method of claim 20, wherein the at least one anticancer agent is selected from tamoxifen, toremifen, raloxifene, droloxifene, iodoxyfene, megestrol acetate, anasfrozole, letrazole, borazole, exemestane, flutamide, nilutamide, bicalutamide, cyproterone acetate, goserelin acetate, luprolide, finasteride, herceptin, methotrexate, 5-fluorouracil, cytosine arabinoside, doxorubicin, daunomycin, epirubicin, idarubicin, mitomycin-C, dactinomycin, mithramycin, cisplatin, carboplatin, melphalan, chlorambucil, busulphan, cyclophosphamide, ifosfamide, nitrosoureas, thiotephan, vincristine, taxol, taxotere, etoposide, teniposide, amsacrine, Irinotecan, topotecan, epothilones, gefitinib, erlotinib, angiogenesis inhibitors, EGF inhibitors, VEGF inhibitors, CDK inhibitors, cytokines, Her1 and Her2 inhibitors, and monoclonal antibodies.


SEQUENCE LISTING (43 sequences)

SEQ ID NO: 1 - protein, 30 aa, Artificial Sequence
  synthetic peptide
YADAIFTNSYRKVLGQLSARKLLQDIMSRQ

SEQ ID NO: 2 - protein, 29 aa, Artificial Sequence
  Synthetic peptide
XADAIXTQSYRXVLXQLSARXLLQDIXDX

SEQ ID NO: 3 - protein, 29 aa, Artificial Sequence
  Synthetic peptide
XADAIXTASYRXVLXQLSARXLLQDIXDX

SEQ ID NO: 4 - protein, 29 aa, Artificial Sequence
  Synthetic peptide
XADAIXTNSYRXVLXQLSARXLLQDIXDX

SEQ ID NO: 5 - protein, 29 aa, Artificial Sequence
  Synthetic peptide
XADAIXTTSYRXVLXQLSARXLLQDIXDX

SEQ ID NO: 6 - protein, 29 aa, Artificial Sequence
  Synthetic peptide
XADAIFTQSYRXVLXQLSARXLLQDIXDX

SEQ ID NO: 7 - protein, 29 aa, Artificial Sequence
  Synthetic peptide
XADAIFTNSYRXVLXQLSARXLLQDIXDX

SEQ ID NO: 8 - protein, 29 aa, Artificial Sequence
  Synthetic peptide
XADAIFTTSYRXVLXQLSARXLLQDIXDX

SEQ ID NO: 9 - protein, 29 aa, Artificial Sequence
  Synthetic peptide
XADAIFTASYRXVLXQLSARXLLQDIXDX

SEQ ID NO: 10 - protein, 29 aa, Artificial Sequence
  Synthetic peptide
XADAIFTQSYRXVLXQLSARXLLQDIXSX

SEQ ID NO: 11 - protein, 29 aa, Artificial Sequence
  Synthetic peptide
XADAIFTNSYRXVLXQLSARXLLQDIXSX

SEQ ID NO: 12 - protein, 29 aa, Artificial Sequence
  Synthetic peptide
XADAIFTTSYRXVLXQLSARXLLQDIXSX

SEQ ID NO: 13 - protein, 29 aa, Artificial Sequence
  Synthetic peptide
XADAIFTASYRXVLXQLSARXLLQDIXSX

SEQ ID NO: 14 - protein, 30 aa, Artificial Sequence
  Synthetic peptide
XADAIFTNSYRXVLXQLSARXLLQDIXDRX

SEQ ID NO: 15 - protein, 30 aa, Artificial Sequence
  Synthetic peptide
XADAIFTQSYRXVLXQLSARXLLQDIXDRX

SEQ ID NO: 16 - protein, 30 aa, Artificial Sequence; identical to SEQ ID NO: 15
  Synthetic peptide

SEQ ID NO: 17 - protein, 30 aa, Artificial Sequence; identical to SEQ ID NO: 14
  Synthetic peptide

SEQ ID NO: 18 - protein, 30 aa, Artificial Sequence
  Synthetic peptide
XADAIFTNSYRXVLXQLSARXLLQDIXSRX

SEQ ID NO: 19 - protein, 30 aa, Artificial Sequence
  Synthetic peptide
XADAIFTTSYRXVLXQLSARXLLQDIXDRX

SEQ ID NO: 20 - protein, 30 aa, Artificial Sequence
  Synthetic peptide
XADAIFTASYRXVLXQLSARXLLQDIXDRX

SEQ ID NO: 21 - protein, 30 aa, Artificial Sequence; identical to SEQ ID NO: 15
  Synthetic peptide

SEQ ID NO: 22 - protein, 30 aa, Artificial Sequence; identical to SEQ ID NO: 14
  Synthetic peptide

SEQ ID NO: 23 - protein, 30 aa, Artificial Sequence; identical to SEQ ID NO: 19
  Synthetic peptide

SEQ ID NO: 24 - protein, 30 aa, Artificial Sequence; identical to SEQ ID NO: 20
  Synthetic peptide

SEQ ID NO: 25 - protein, 30 aa, Artificial Sequence; identical to SEQ ID NO: 15
  Synthetic peptide

SEQ ID NO: 26 - protein, 30 aa, Artificial Sequence; identical to SEQ ID NO: 14
  Synthetic peptide

SEQ ID NO: 27 - protein, 30 aa, Artificial Sequence; identical to SEQ ID NO: 19
  Synthetic peptide

SEQ ID NO: 28 - protein, 30 aa, Artificial Sequence; identical to SEQ ID NO: 20
  Synthetic peptide

SEQ ID NO: 29 - protein, 30 aa, Artificial Sequence; identical to SEQ ID NO: 15
  Synthetic peptide

SEQ ID NO: 30 - protein, 30 aa, Artificial Sequence; identical to SEQ ID NO: 15
  Synthetic peptide

SEQ ID NO: 31 - protein, 30 aa, Artificial Sequence
  Synthetic peptide
XXDAIFTNSYRXVLXQLSARXLLQDIXSRX

SEQ ID NO: 32 - protein, 29 aa, Artificial Sequence
  Synthetic peptide
XADAIFTQSYRXVLXQLSARXLLQDIXSX

SEQ ID NO: 33 - protein, 29 aa, Artificial Sequence
  Synthetic peptide
XADAIFTQSYRXVLXQLSARXLLQDIXDX

SEQ ID NO: 34 - protein, 29 aa, Artificial Sequence
  Synthetic peptide
XADAIFTQSYRXVLXQLSARXLLQDIXDX

SEQ ID NO: 35 - protein, 29 aa, Artificial Sequence
  Synthetic peptide
XADAIFTQSYHXVLXQLSAHXLLQDIXDX

SEQ ID NO: 36 - protein, 30 aa, Artificial Sequence; identical to SEQ ID NO: 18
  Synthetic peptide

SEQ ID NO: 37 - protein, 29 aa, Artificial Sequence
  Synthetic peptide
XADAIFTTSYRXVLXQLSARXLLQDIXSX

SEQ ID NO: 38 - protein, 29 aa, Artificial Sequence
  Synthetic peptide
XADAIFTTSYRXVLXQLSARXLLQDIXSX

SEQ ID NO: 39 - protein, 30 aa, Artificial Sequence
  Synthetic peptide
XADAIFTQSYRXVLXQLSARXLLQDIXSRX

SEQ ID NO: 40 - protein, 29 aa, Artificial Sequence
  Synthetic Peptide
XADAIFTNSYRXVLXQLSARXLLQDIXDX

SEQ ID NO: 41 - protein, 29 aa, Artificial Sequence
  Synthetic Peptide
XADAIFTTSYRXVLXQLSARXLLQDIXDX

SEQ ID NO: 42 - protein, 29 aa, Artificial Sequence
  Synthetic peptide
XADAIFTQSYRXVLXQLSARXLLQDIXDX

SEQ ID NO: 43 - protein, 44 aa, Artificial Sequence
  Synthetic peptide
YADAIFTNSYRKVLGQLSARKLLQDIMSRQQGESNQERGARARL